(19) United States
(12) Patent Application Publication
(10) Pub. No.: US 2008/0213817 A1
(43) Pub. Date: Sep. 4, 2008

(54) Enhancement of Th2-Dependent and Inflammatory Response

(76) Inventors: Michael Karin, La Jolla, CA (US); Min Gao, Edison, NJ (US); Tord Labuda, Malmo (SE)

Correspondence Address: Medlen & Carroll, Suite 350, 101 Howard Street, San Francisco, CA, 94105, US

(21) Appl. No.: 10/590,042

(22) PCT Filed: Feb. 17, 2005
(86) PCT No.: PCT/US2005/005066

§ 371 Date: Jun. 27, 2007

Related U.S. Application Data
(60) Provisional application No. 60/546,540, filed on Feb. 19, 2004.

(51) Int. Cl. C12Q 1/02; C12N 5/06

(52) U.S. Cl. 435/29; 435/375

(57) ABSTRACT

The present invention relates to altering the levels of Th2 cytokine production, and in particular, biasing the cytokine expression profile towards Th2 cytokine production through mitogen-activated protein kinase/ERK kinase kinase 1 (MEKK1), the screening of agents that increase Th2 cytokine production, and the treatment of Th1 associated autoimmune diseases in vivo. In one embodiment, the present invention relates to agents including but not limited to reducing the activity of MEKK1, leading to increased levels of Th2 cytokine production.

### GOVERNMENT INTERESTS

The present invention was funded by grants from the National Institutes of Health, number R21AI48542, ES04151, AI43477, and ES06376. As such, the United States Government may have certain rights to this invention.

### FIELD OF THE INVENTION

The present invention relates to altering the levels of Th2 cytokine production, and in particular, biasing the cytokine expression profile towards Th2 cytokine production through mitogen-activated protein kinase/ERK kinase kinase 1 (MEKK1), the screening of agents that increase Th2 cytokine production, and the treatment of Th1 associated autoimmune diseases in vivo. In one embodiment, the present invention relates to agents including but not limited to reducing the activity of MEKK1, leading to increased levels of Th2 cytokine production.

### BACKGROUND

Debilitating autoimmune disorders are associated with chronically activated T cell populations producing Type 1 pro-inflammatory cytokines (Th1 populations). These autoimmune disorders are classified as Th1 or type 1 disorders and include type 1 diabetes, autoimmune thyroiditis, etc. One therapeutic mechanism is to treat these disorders with cytokines such as IL-10, IL-4, and IL-11 typically considered type 2 cytokines produced by T cells categorized as but not limited to Th2 populations. In vitro, the presence of Th2 cytokines may counter or reduce the production of type 1 cytokines in addition to inhibiting farther Th1 maturation or biasing further maturation of T cells towards Th2 cytokine production. However, although these cytokine treatments show promise for certain diseases, these treatments are expensive and require frequent injections or infusions.

Increased cytokine production occurs when T cell receptor (TCR) engagement induces activation of several transcription factors, including AP-1 that in turn activates a related network of signal transduction molecules including numerous MAP kinases, JNK molecules and ubiqases. The intensity or duration of T cell activation can bias the cytokine expression profile towards Th1 like or Th2 like, but the underlying biochemical mechanism is unknown.

Thus, there is a need to determine the underlying biochemical mechanism for augmenting Th2 cytokine production in order to find new ways to identify drugs that will reduce chronically activated Th1 cell populations, and in particular augment signaling pathways that produce Th2 cytokines.

### SUMMARY OF THE INVENTION

The present invention relates to altering the levels of Th2 cytokine production, and in particular, biasing the cytokine expression profile towards Th2 cytokine production through mitogen-activated protein kinase/ERK kinase kinase 1 (MEKK1), the screening of agents that increase Th2 cytokine production, and the treatment of Th1 associated autoimmune diseases in vivo. In one embodiment, the present invention relates to agents including but not limited to reducing the activity of MEKK1, leading to increased levels of Th2 cytokine production.

In one embodiment, the prevention and treatment of diseases known to be associated with high T helper 1 (Th1) cell cytokine levels is accomplished by interfering with the MEKK1-ITCH cascade pathway and interactions as described herein to increase the levels of T helper 2 (Tc2) cytokines. In one embodiment, the prevention and treatment of diseases known to be associated with Th1 cell levels is accomplished by interfering with the MEKK1-ITCH cascade pathway and interactions as described herein to increase the production of Th2 cells. In certain embodiments, the importance of MEKK1-ITCH cascade pathway and the MEKK1-ITCH interactions with the use of MEKK1 and JNK knockout mouse models and the use of JNK and other inhibitors are described. These embodiments serve to distinguish agents that would be drug candidates as anti-inflammatory for treatment of Th1 cytokine disorders.

In one embodiment, the invention provides a method for identifying a test agent as reducing the level of differentiation of T cells into T helper type 1 (Th1) cells comprising reducing mitogen-activated protein kinase/ERK kinase kinase 1 catalytic activity in said T cells. In another embodiment, the invention provides a method for identifying a test agent wherein reducing of said mitogen-activated protein kinase/ERK kinase kinase 1 catalytic activity comprises increasing the level of differentiation of said T cells into T helper type 2 (Th2) cells. In another embodiment, the invention provides a method for identifying a test agent wherein the reducing of said mitogen-activated protein kinase/ERK kinase kinase 1 catalytic activity comprises increasing the level of one or more T helper type 2 (Th2) cell cytokine that is produced by said T cell. In another embodiment, the invention provides a method for identifying a test agent wherein the increased level of said Th2 cytokine occurs in the absence of an increase in the level of one or more T helper type 1 (Th1) cytokine. In another embodiment, the invention provides a method for identifying a test agent wherein said Th1 cytokine is chosen from one or more of interferon-gamma and Interleukin-2. In another embodiment, the invention provides a method for identifying a test agent wherein said Th2 cytokine is chosen from one or more of Interleukin-4, Interleukin-5, Interleukin-10, and Interleukin-13. In another embodiment, the invention provides a method for identifying a test agent wherein said increasing the level of said Th2 cell cytokine comprises increasing the level of mRNA encoding said Th2 cytokine. In another embodiment, the invention provides a method for identifying a test agent wherein said mRNA encoding said Th2 cytokine is increased 5 fold. In another embodiment, the invention provides a method for identifying a test agent wherein said reducing of said mitogen-activated protein kinase/ERK kinase kinase 1 catalytic activity comprises increasing the level of proliferation of T helper type 2 (Th2) cells that differentiate from said T cells. In another embodiment, the invention provides a method for identifying a test agent wherein said reducing of said mitogen-activated protein kinase/ERK kinase kinase 1 catalytic activity comprises introducing a mutation in the gene encoding mitogen-activated protein kinase/ERK kinase kinase 1. In another embodiment, the invention provides a method for identifying a test agent wherein said T cells comprise thymocyte cells. In another embodiment, the invention provides a method for identifying a test agent wherein said T cells comprise splenocyte cells. In another embodiment, the invention provides a method for identifying a test agent wherein said T cells are in vitro. In another embodiment, the invention provides a method for identifying a test agent wherein said T cells are in vivo in an animal. In another embodiment, the invention provides a method for identifying a test agent wherein said animal is human. In another embodiment, the invention provides a method for identifying a test agent wherein said human is chosen from a human that is: (a) suspected of having a Th1-mediated disease; (b) not suspected of having a Th1-mediated disease; (c) suspected of being capable of developing a Th1-mediated disease; and (d) suspected of not being capable of developing a Th1-mediated disease. In another embodiment, the invention provides a method for identifying a test agent wherein said Th1-mediated disease is chosen from multiple sclerosis, type 1 diabetes, autoimmune thyroiditis, and rheumatoid arthritis.

In one embodiment, the present invention provides a method for identifying a test agent as reducing the level of differentiation of T cells into T helper type 1 (Th1) cells, comprising: a) providing: i) a test agent; and ii) mitogen-activated protein kinase/ERK kinase kinase 1; and b) contacting said test agent and said mitogen-activated protein kinase/ERK kinase kinase 1; and c) detecting reduced mitogen-activated protein kinase/ERK kinase kinase 1 kinase activity in the presence of said agent compared to in the absence of said agent, thereby identifying said test agent as causing one or more of increasing Th2 cells, decreasing the level of Th1 cells, and decreasing Th1 disease. In another embodiment, the invention provides a method for identifying a test agent wherein said method comprises one or more of: (a) identifying said agent as increasing the level of differentiation of said T cells into T helper type 2 (Th2) cells; (b) identifying said agent as increasing the level of one or more T helper type 2 (Th2) cell cytokine that is produced by said T cell; and (c) identifying said agent as increasing the level of proliferation of T helper type 2 (Th2) cells that differentiate from said T cells.

In one embodiment, the present invention provides a method for increasing Th2 cytokine levels produced by T cells, comprising: (a) providing: (i) an inhibitor of E3 ubiquitin ligase itch; (ii) T cells; (iii) test agent; and (b) contacting said T cells in the presence of said test agent to produce contacted T cells and in the absence of said test agent to produce control T cells; and (c) detecting reduced activity of E3 ubiquitin ligase itch in said contacted T cells compared to E3 ubiquitin ligase itch in said control T cells, wherein said detecting identifies said test agent as increasing Th2 cytokine levels produced by T cells.

In one embodiment, the present invention provides a method for increasing Th2 cytokine levels produced by T cells, comprising: (a) providing: (i) a kinase inhibitor, wherein said kinase is one or more of mitogen-activated protein kinase/ERK kinase kinase 1 and C-Jun N-terminal kinase 1; (ii) T cells; (iii) test agent; and (b) contacting said T cells in the presence of said test agent to produce contacted T cells and in the absence of said test agent to produce control T cells; and (c) detecting reduced activity of said kinase in said contacted T cells compared to said kinase in said control T cells, wherein said detecting identifies said test agent as increasing Th2 cytokine levels produced by T cells. In another embodiment, the invention provides a method further comprising, (d) identifying said test agent as increasing the level of one or more of Th2 cytokine. In another embodiment, the invention provides a method wherein said Th2 cytokine is one or more of Interleukin-4, Interleukin-5, Interleukin-10, and Interleukin-13. In another embodiment, the invention provides a method further comprising, (d) identifying said test agent as decreasing the level of Th1 cytokines. In another embodiment, the invention provides a method wherein said kinase inhibitor comprises SB600125.

In one embodiment, the present invention provides a method for increasing Th2 cytokine levels produced by T cells, comprising reducing the activity of a E3 ubiquitin ligase itch.

In one embodiment, the present invention provides a method for increasing Th2 cytokine levels produced by T cells, comprising reducing the activity of a mitogen-activated protein kinase/ERK kinase kinase 1.

In one embodiment, the present invention provides a method for increasing Th2 cytokine levels produced by T cells, comprising reducing the activity of a C-Jun N-terminal kinase 1.

In one embodiment, the present invention provides a method for increasing Th2 cytokine levels produced by T cells, comprising: (a) providing: (i) T cells; and (ii) agent that reduces activity of E3 ubiquitin ligase itch; and (b) contacting said T cells with said agent under conditions such that said agent reduces said activity of said E3 ubiquitin ligase itch.

In one embodiment, the present invention provides a method for increasing Th2 cytokine levels produced by T cells, comprising: (a) providing: (i) T cells; and (ii) agent that reduces activity of a kinase, wherein said kinase is one or more of mitogen-activated protein kinase/ERK kinase kinase 1 and C-Jun N-terminal kinase 1; and (b) contacting said T cells with said agent and without said agent under conditions such that said agent reduces activity of said kinase. In another embodiment, the invention provides a method for increasing Th2 cytokine levels produced by T cells further comprising (c) identifying said test agent as comprising increasing the level of Interleukin-10 produced by said T cells. In another embodiment, the invention provides a method for increasing Th2 cytokine levels produced by T cells wherein said T cells are inflammatory disease T cells. In another embodiment, the invention provides a method for increasing Th2 cytokine levels produced by T cells wherein said inflammatory disease is one or more of type 1 diabetes, autoimmune thyroiditis, multiple sclerosis and rheumatoid arthritis.

In one embodiment, the present invention provides a method for increasing Th2 cytokine levels produced by pro-inflammatory disease T cells, comprising: (a) providing: (i) pro-inflammatory disease T cells; and (ii) agent that reduces activity of E3 ubiquitin ligase itch; and (b) contacting said pro-inflammatory disease T cells with said agent under conditions such that said agent increases the level of Interleukin-10 produced by said T cells.

In one embodiment, the present invention provides a method for reducing inflammation and disease associated with Th1 cell abundance by increasing the in vivo production of Th2 cells comprising reducing one or more of MEKK1-MEKK4/MEKK7-JNK-ITCH cascade pathway activity and direct MEKK1-ITCH protein to protein interactions. In another embodiment, the present invention provides a method to reduce inflammation and disease associated with Th1 cell abundance wherein said reducing is comprises using one or more MEKK1 enzyme inhibitors. In another embodiment, the present invention provides a method to reduce inflammation and disease associated with Th1 cell abundance wherein said enzyme inhibitors comprises SP600125. In another embodiment, the present invention provides a method to reduce inflammation and disease associated with Th1 cell abundance wherein said reducing comprises using one or more neutralizing antibodies that specifically bind to MEKK1. In another embodiment, the present invention provides a method to reduce inflammation and diseases associated with Th1 cell abundance wherein said reducing is achieved by reducing expression of the MEKK1 gene. In another embodiment, the present invention provides a method to reduce inflammation and disease associated with Th1 cell abundance wherein said reducing comprises using one or more MEKK4/MEKK7 enzyme inhibitors. In another embodiment, the present invention provides a method to reduce inflammation and disease associated with Th1 cell abundance wherein said MEKK4/MEKK7 enzyme inhibitors comprises SP600125. In another embodiment, the present invention provides a method to reduce inflammation and disease associated with Th1 cell abundance wherein said reducing comprises neutralizing antibodies that specifically bind to MEKK4/MEKK7. In another embodiment, the present invention provides a method to reduce inflammation and disease associated with Th1 cell abundance wherein said reducing is comprises inhibiting expression of the MEKK4/MEKK7 gene. In another embodiment, the present invention provides a method to reduce inflammation and disease associated with Th1 cell abundance wherein said reducing comprises using ITCH enzyme inhibitors. In another embodiment, the present invention provides a method to reduce inflammation and disease associated with Th1 cell abundance wherein said reducing comprises using neutralizing antibodies that specifically bind to ITCH. In another embodiment, the present invention provides a method to reduce inflammation and disease associated with Th1 cell abundance wherein said reducing comprises inhibiting the expression of the itch gene. In another embodiment, the present invention provides a method to reduce inflammation and disease associated with Th1 cell abundance wherein said inhibitor comprises SP600125. In another embodiment, the present invention provides a method to reduce inflammation and disease associated with Th1 cell abundance wherein the expression of the JNK gene is suppressed by the use of one or more of RNAi, and antisense molecules. In another embodiment, the present invention provides a method to reduce inflammation and disease associated with Th1 cell abundance wherein the expression of the MEKK1 gene is suppressed by the use of one or more of RNAi and antisense molecules. In another embodiment, the present invention provides a method to reduce inflammation and disease associated with Th1 cell abundance wherein the expression of the MEKK4/MKK7 gene is suppressed by the use of one or more of RNAi and antisense molecules. In another embodiment, the present invention provides a method to reduce inflammation and disease associated with Th1 cell abundance wherein the expression of the Itch gene is suppressed by the use of one or more of RNAi and antisense molecules. In another embodiment, the present invention provides a method to reduce inflammation and disease associated with Th1 cell abundance by increasing the in vivo production of Th2 cells wherein the neutralizing antibody is chosen from human antibody and humanized antibody that invoke minimum and therapeutically acceptable level of immunogenic defense response in a human. In another embodiment, the present invention provides a method to reduce inflammation and disease associated with Th1 cell abundance by increasing the in vivo production of Th2 cells wherein the MEKK1-Itch interactions are reduced by using SP600125. In another embodiment, the present invention provides a method to reduce inflammation and disease associated with Th1 cell abundance wherein MEKK1-Itch interactions are reduced by the use of neutralizing antibodies against one or more of MEKK1 and Itch. In another embodiment, the present invention provides a method to reduce inflammation and disease associated with Th1 cell abundance by increasing the in vivo production of Th2 cells wherein the neutralizing antibody is chosen from human antibody and humanized antibody that invoke minimum and therapeutically acceptable level of immunogenic defense response in a human.

In one embodiment, the present invention provides a composition comprising a transgenic mouse that comprises MEKK1-/MEKK1− or MEKK1-/MEKK1+.

In one embodiment, the present invention provides a method for identifying therapeutic agents that are useful in reducing one or more of MEKK1-MEKK4/MEKK7-JNK-ITCH cascade pathway activity and direct MEKK1-ITCH protein to protein interactions comprising: (a) providing; WT and Mekk1^{KD} thymocytes stimulated with anti-CD3 and anti-CD28 for 24 hrs in the absence or presence (0.5 mM) of a JNK inhibitor; (b) preparing cell lysates from said thymocytes; (c) immunoblotting said lysates; and (d) determining levels of one or more of Itch, c-Jun and JunB to identify therapeutic agents that are useful in reducing cascade pathway activity.

In one embodiment, the present invention provides a method to reduce inflammation and disease associated with Th1 cell abundance by increasing the in vivo production of Th2 cells. In another embodiment, the present invention provides a method wherein said JNK inhibitor comprises SP600125. In another embodiment, the present invention provides a method for reducing inflammation and disease associated with Th1 cell abundance by increasing the in vivo production of Th2 cells wherein said disease is chosen from multiple sclerosis, type 1 diabetes, autoimmune thyroiditis, and rheumatoid arthritis

In one embodiment, the present invention provides a method for reducing the symptoms of type 1 diabetes by increasing in vivo production of Th2 cells comprising reducing one or more of MEKK1-MEKK4/MEKK7-JNK-ITCH cascade pathway activity and direct MEKK1-ITCH protein to protein interactions.

In one embodiment, the present invention provides a method for reducing the symptoms of autoimmune thyroiditis by increasing in vivo production of Th2 cells comprising reducing one or more of MEKK1-MEKK4/MEKK7-JNK-ITCH cascade pathway activity and direct MEKK1-ITCH protein to protein interactions.

In one embodiment, the present invention provides a method for reducing the symptoms of multiple sclerosis by increasing in vivo production of Th2 cells comprising reducing one or more of MEKK1-MEKK4/MEKK7-JNK-ITCH cascade pathway activity and direct MEKK1-ITCH protein to protein interactions.

### BRIEF DESCRIPTION OF THE DRAWINGS

FIG. 1 shows exemplary embodiments demonstrating hyperproliferation of Mekk1^{KD} mutant T cells.

FIG. 2 shows exemplary embodiments demonstrating a reduced JNK activity results in increased thymocyte proliferation.

FIG. 3 shows an exemplary embodiment demonstrating enhanced Th2 cytokine production by Mekk1^{KD} thymocytes.

FIG. 4 shows an exemplary embodiment demonstrating upregulation of JunB and c-Jun protein levels in Mekk1^{KD} thymocytes.

FIG. 5 shows an exemplary embodiment demonstrating MEKK1 promotes ubiquitin-dependent degradation of c-Jun and JunB.

FIG. 6 shows an exemplary embodiment demonstrating MEKK1/JNK signaling pathway regulates Itch and Jun turnover.

FIG. 7 shows an exemplary embodiment demonstrating MEKK1/JNK signaling pathway negatively and positively regulates JunB stability and activity in response to T cell stimulation.

FIG. 8 shows an exemplary embodiment demonstrating a reduced JNK activity and increased T cell proliferation in Mekk1^{ΔKD} T cells.

FIG. 9 shows an exemplary embodiment demonstrating enhanced Th2 cytokine production and skewed differentiation by Mekk1^{ΔKD} T cells.

FIG. 10 shows an exemplary embodiment demonstrating inactivation of MEKK1 or JNK slows down JunB and c-Jun turnover.

FIG. 11 shows an exemplary embodiment demonstrating the MEKK1-JNK cascade enhances the E3 ubiquitin ligase activity of Itch.

FIG. 12 shows an exemplary embodiment demonstrating that MEKK1-JNK cascade promotes Itch-dependent c-Jun and JunB ubiquitination in living cells.

FIG. 13 shows an exemplary embodiment demonstrating Co-stimulation of T cells enhancing JunB turnover.

FIG. 14 shows an exemplary embodiment demonstrating JunB turnover according to the strength of the T cell activating stimulus summarized as a model summarizing these results and illustrating a mechanism through which the MEKK1-JNK signaling pathway can modulate the extent of Th2 differentiation.

FIG. 15 shows an exemplary embodiment demonstrating increased Th2 cytokine production in Mekk1^{ΔKD} cells with reduced JNK activity.

FIG. 16 shows an exemplary embodiment demonstrating a decreased turnover of JunB and c-Jun after inactivation of MEKK1 or JNK.

FIG. 17 shows an exemplary embodiment demonstrating a MEKK1-JNK cascade promotes c-Jun and JunB ubiquitination by enhancing Itch activity.

FIG. 18 shows an exemplary embodiment demonstrating increased E3 activity of Itch after JNK-mediated phosphorylation.

### DEFINITIONS

To facilitate understanding of the invention, a number of terms are defined below.

As used herein including within this specification and the appended claims, the forms “a,” “an” and “the” includes both singular and plural references unless the content clearly dictates otherwise.

As used herein, the term “or” when used in the expression “A or B,” and where A and B refer to a composition, disease, product, etc., means one, or the other, or both.

As used herein, the term “comprising” when placed before the recitation of steps in a method means that the method encompasses one or more steps that are additional to those expressly recited, and that the additional one or more steps may be performed before, between, and/or after the recited steps. For example, a method comprising steps a, b, and c encompasses a method of steps a, b, x, and c, a method of steps a, b, c, and x, as well as a method of steps x, a, b, and c. Furthermore, the term “comprising” when placed before the recitation of steps in a method does not (although it may) require sequential performance of the listed steps, unless the content clearly dictates otherwise. For example, a method comprising steps a, b, and c encompasses, for example, a method of performing steps in the order of steps a, c, and b, the order of steps c, b, and a, and the order of steps c, a, and b, etc.

As used herein, the term “inflammation” refers to a response of redness, swelling, pain, and a feeling of heat in certain areas that includes but is not limited to an inflammatory response. Inflammation can include but not be limited to characteristics of increased blood flow and entry of leukocytes into the tissues, resulting in swelling, redness, elevated temperature and pain.

As used herein, “pro-inflammatory” and “inflammatory” refers to but is not limited to cells, molecules, signaling pathways, etc. that induce and support immune responses that are not limited to and include T1 responses.

As used herein, the term “inflammatory cells” refers to a collection of immune system cells and molecules that invade tissues and organs as part of an immune system response.

As used herein, “immune response” refers to one or more of cell-mediated, humoral and the like.

As used herein, “cell mediated response” refers to an immune response that predominantly involves immune cell activation.

As used herein, “humoral” and “humoral” response refers to an immune response that predominantly involves antibody production.

The terms “antibody” and “immunoglobulin” are interchangeably used to refer to a glycoprotein or a portion thereof (including single chain antibodies), which is evoked in an animal by an immunogen and which demonstrates specificity to the immunogen, or, more specifically, to one or more epitopes contained in the immunogen. The term “antibody” includes polyclonal antibodies, monoclonal antibodies, blocking antibodies, neutralizing antibodies, inhibiting antibodies, stimulating antibodies, naturally occurring antibodies as well as non-naturally occurring antibodies, including, for example, single chain antibodies, chimeric, bifunctional and humanized antibodies, as well as antigen-binding fragments thereof, including, for example, Fab, F(ab′)₂, Fab fragments, Fd fragments, and Ev fragments of an antibody, as well as a Fab expression library. It is intended that the term “antibody” encompass any immunoglobulin (e.g., IgG, IgM, IgA, IgE, IgD, etc.) obtained from any source (e.g., humans, rodents, non-human primates, caprines, bovines, equines, ovines, etc.). The term “polyclonal antibody” refers to an immunoglobulin produced from more than a single clone of plasma cells; in contrast “monoclonal antibody” refers to an immunoglobulin produced from a single clone of plasma cells. Monoclonal and polyclonal antibodies may or may not be purified. For example, polyclonal antibodies contained in crude antiserum may be used in this unpurified state.

In one embodiment, the agent that reduces activity of mitogen-activated protein kinase (MAP)/ERK kinase kinase 1 (MEKK1), C-Jun N-terminal kinase 1 (JNK1), itch, etc., is an antibody, such as MEKK1 or JNK1 or ITCH or MEKK1/MEKK7 complex or MEKK1/MEEK4/MEEK1 complex peptide antibody, and/or MEKK1 or JNK1 or ITCH sequence antibody. An example of a MAP kinase kinase antibody, herein incorporated by reference in U.S. Pat. No. 6,465,618, Nishida, et al. (Oct. 15, 2002).

Naturally occurring antibodies may be generated in any species including murine, rat, rabbit, hamster, human, and simian species using methods known in the art. Non-naturally occurring antibodies can be constructed using solid phase peptide synthesis, can be produced recombinantly or can be obtained, for example, by screening combinatorial libraries consisting of variable heavy chains and variable light chains as previously described [Huse et al., Science 246:1275-1281 (1989)]. These and other methods of making, for example, chimeric, humanized, CDR-grafted, single chain, and bifunctional antibodies are well known to those skilled in the art (Winter and Harris, Immunol. Today 14:243-246 (1993); Ward et al., Nature 341:544-546 (1989); Hilyard et al., Protein Engineering: A practical approach (IRL Press 1992); and Borrabeck, Antibody Engineering, 2d ed. (Oxford University Press 1995).

Those skilled in the art know how to make polyclonal and monoclonal antibodies, which are specific to a desirable polypeptide. For the production of monoclonal and polyclonal antibodies, various host animals can be immunized by injection with the peptide corresponding to any molecule of interest in the present invention, including but not limited to rabbits, mice, rats, sheep, goats, chickens, etc. In one preferred embodiment, the peptide is conjugated to an immunogenic carrier (e.g., diphtheria toxoid, bovine serum albumin (BSA), or keyhole limpet hemocyanin (KLH)). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum.

For preparation of monoclonal antibodies directed toward molecules of interest in the present invention, any technique that provides for the production of antibody molecules by continuous cell lines in culture may be used (See, e.g., Harlow and Lane, Antibodies: A Laboratory Manual, Cold Spring Harbor, Laboratory Press, Cold Spring Harbor, N.Y.). These include but are not limited to the hybridoma technique originally developed by Köhler and Milstein (Köhler and Milstein, Nature 256:495-497 [1975]), as well as the trioma technique, the human B-cell hybridoma technique (See e.g. Kozbor et al. Immunol. Today 4:72 [1983]), and the EBV-hybridoma technique to produce human monoclonal antibodies (Cole et al., in Monoclonal Antibodies and Cancer Therapy, Alan R. Liss, Inc., pp. 77-96 [1985]). In some particularly preferred embodiments of the present invention, the present invention provides monoclonal antibodies of the IgG class.

In additional embodiments of the invention, monoclonal antibodies can be produced in germ-free animals utilizing technology such as that described in PCT/US90/02545. In addition, human antibodies may be used and can be obtained by using human hybridomas (Cote et al., Proc. Natl. Acad. Sci. U.S.A. 80: 2026-2030 [1983]) or by transforming human B cells with EBV virus in vitro (Cole et al., in Monoclonal Antibodies and Cancer Therapy, Alan R. Liss, pp. 77-96 [1985]).

Furthermore, techniques described for the production of single chain antibodies (See e.g., U.S. Pat. No. 4,946,778; herein incorporated by reference) can be adapted to produce single chain antibodies that specifically recognize a molecule of interest (e.g., at least a portion of an AUBP or mammalian exosome, as described herein). An additional embodiment of the invention utilizes the techniques described for the construction of Fab expression libraries (Huse et al., Science 246:1275-1281 [1989]) to allow rapid and easy identification of monoclonal Fab fragments with the desired specificity for a particular protein or epitope of interest (e.g., at least a portion of an AUBP or mammalian exosome).

The invention also contemplates humanized antibodies. Humanized antibodies may be generated using methods known in the art, including those described in U.S. Pat. Nos. 5,545,806; 5,569,825 and 5,625,126, the entire contents of which are incorporated by reference. Such methods include, for example, generation of transgenic non-human animals which contain human immunoglobulin chain genes and which are capable of expressing these genes to produce a repertoire of antibodies of various isotypes encoded by the human immunoglobulin genes.

According to the invention, techniques described for the production of single chain antibodies (U.S. Pat. No. 4,946,778; herein incorporated by reference) can be adapted to produce specific single chain antibodies as desired. An additional embodiment of the invention utilizes the techniques known in the art for the construction of Fab expression libraries (Huse et al., Science, 246:1275-1281 [1989]) to allow rapid and easy identification of monoclonal Fab fragments with the desired specificity.

Antibody fragments that contain the idiotype (antigen binding region) of the antibody molecule can be generated by known techniques. For example, such fragments include but are not limited to: the F(ab′)2 fragment that can be produced by pepsin digestion of an antibody molecule; the Fab′ fragments that can be generated by reducing the disulfide bridges of an F(ab′)2 fragment, and the Fab fragments that can be generated by treating an antibody molecule with papain and a reducing agent.

In the production of antibodies, screening for the desired antibody can be accomplished by techniques known in the art (e.g., radioimmunoassay, ELISA [enzyme-linked immunosorbent assay], “sandwich” immunoassays, immunoradiometric assays, gel diffusion precipitin reactions, immunodiffusion assays, in situ immunoassays [e.g., using colloidal gold, enzyme or radioisotope labels], Western blots, precipitation reactions, agglutination assays (e.g., gel agglutination assays, hemagglutination assays, etc.), complement fixation assays, immunofluorescence assays, protein A assays, and immunoelectrophoresis assays, etc.

In an alternative embodiment, the agent that alters the level of binding of MEKK1 or MEKK4 or MKK7 or JNK1 or itch with a MEKK1 or MEKK4 or MKK7 or JNK1 or itch sequence, respectively, is a nucleic acid sequence. The terms nucleic acid sequence therein refer to two or more nucleotides which are covalently linked to each other. Included within this definition are oligonucleotides, polynucleotide, and fragments or portions thereof, DNA or RNA of genomic or synthetic origin which may be single- or double-stranded, and represent the sense or antisense strand. Nucleic acid sequences which are particularly useful in the instant invention include, without limitation, antisense sequences and ribozymes. In an example herein incorporated by reference, Flavell et al. Aug. 21, 2003 United States Patent Application 20030157539 A1, a nucleic acid inhibitor comprising IRAK-M reduces toll-like receptor signaling.

In one embodiment, the agent that alters the level of MEKK1 or MEKK4 or MKK7 or JNK1 or itch with a MEKK1 or MEKK4 or MKK7 or JNK1 or itch sequence, is an antisense nucleic acid sequence. Antisense sequences have been successfully used to inhibit the expression of several genes [Markus-Sekura (1988) Anal. Biochem. 172:289-295; Hambor et al. (1988) J. Exp. Med. 168:1237-1245; and patent EP 140 308], including the gene encoding VCAM1, one of the integrin 4 1 ligands [U.S. Pat. No. 6,252,043, incorporated in its entirety by reference]. As an example, in U.S. Pat. No. 6,054,440 Monia, et al. (Apr. 25, 2000), herein incorporated by reference, antisense sequences were used to modulate Jun N-terminal Kinase Kinase-2 expression through function of nucleic acid molecules encoding Jun N-terminal Kinase Kinase-2. The terms “antisense DNA sequence” and “antisense sequence” as used herein interchangeably refer to a deoxyribonucleotide sequence whose sequence of deoxyribonucleotide residues is in reverse 5′ to 3′ orientation in relation to the sequence of deoxyribonucleotide residues in a sense strand of a DNA duplex. A “sense strand” of a DNA duplex refers to a strand in a DNA duplex which is transcribed by a cell in its natural state into a “sense mRNA.” Sense mRNA generally is ultimately translated into a polypeptide. Thus, an “antisense DNA sequence” is a sequence which has the same sequence as the non-coding strand in a DNA duplex, and which encodes an “antisense RNA” (i.e., a ribonucleotide sequence whose sequence is complementary to a “sense mRNA” sequence). The designation (−) (i.e., “negative”) is sometimes used in reference to the antisense strand, with the designation (+) sometimes used in reference to the sense (i.e., “positive”) strand. Antisense RNA may be produced by any method, including synthesis by splicing an antisense DNA sequence to a promoter which permits the synthesis of antisense RNA. The transcribed antisense RNA strand combines with natural mRNA produced by the cell to form duplexes. These duplexes then block either the further transcription of the mRNA or its translation, or promote its degradation.

Antisense oligonucleotide sequences may be synthesized using any of a number of methods known in the art (such as solid support and commercially available DNA synthesizers, standard phosphoramidate chemistry techniques, and commercially available services, e.g., Genta, Inc.).

As used herein, the term “RNAi” and “RNA interference” refers to the ability of double stranded RNA to suppress the expression of a gene corresponding to its own sequence.

In some alternative embodiments, the agent that alters the level of T2 cytokines or MEKK1 or MEKK4 or MKK7 or JNK1 or itch and/or T1 cytokines or MEKK1 or MEKK4 or MKK7 or JNK1 or itch is a ribozyme nucleic acid sequence. Ribozyme sequences have been successfully used to inhibit the expression of several genes including the gene encoding VCAM1, which is one of the integrin 4 1 ligands [U.S. Pat. No. 6,252,043, incorporated in its entirety by reference]. The term “ribozyme” refers to an RNA sequence that hybridizes to a complementary sequence in a substrate RNA and cleaves the substrate RNA in a sequence specific manner at a substrate cleavage site. Typically, a ribozyme contains a “catalytic region” flanked by two “binding regions.” The ribozyme binding regions hybridize to the substrate RNA, while the catalytic region cleaves the substrate RNA at a “substrate cleavage site” to yield a “cleaved RNA product.” Examples of ribosomes that modulate genes related to apoptosis are NF-κβ genes, such as REL-A, REL-B, REL (c-rel), NF-κβ1 (p105/p50) and NF-κβ2 (p100)/p52/p49), herein incorporated by reference, are demonstrate in United States Patent Application, 20020177568 A1, Stinchcomb, et al., Nov. 28, 2002.

Molecules which find use as agents for specifically altering the level of specific binding of MEKK1 or MEKK4 or MKK7 or JNK1 or itch with effector molecule sequences include organic molecules, inorganic molecules, and libraries of any type of molecule, which can be screened using a method of the invention, and which may be prepared using methods known in the art. These agents are made by methods for preparing oligonucleotide libraries [Gold et al., U.S. Pat. No. 5,270,163, incorporated by reference]; peptide libraries [Koivunen et al. J. Cell Biol., 124: 373-380 (1994)]; peptidomimetic libraries [Blondelle et al., Trends Anal. Chem. 14:83-92 (1995)] oligosaccharide libraries [York et al., Carb. Res. 285:99-128 (1996); Liang et al., Science 274:1520-1522 (1996); and Ding et al., Adv. Expt. Med. Biol. 376:261-269 (1995)]; lipoprotein libraries [de Kruif et al., FEBS Lett., 399:232-236 (1996)]; glycoprotein or glycolipid libraries [Karaoglu et al., J. Cell Biol. 130:567-577 (1995)]; or chemical libraries containing, for example, drugs or other pharmaceutical agents [Gordon et al., J. Med. Chem. 37:1385-1401 (1994); Ecker and Crook, Bio/Technology 13:351-360 (1995), U.S. Pat. No. 5,760,029, incorporated by reference]. Libraries of diverse molecules also can be obtained from commercial sources.

As used herein, the term “T cell” refers to lymphocytes that differentiate primarily in the thymus and are central to the control and development of immune responses. Examples of T cells include but are not limited to T helper cells (e.g. Th0, Th1 and Th2 and the like) and cytotoxic T cells (e.g. Tc1 and Tc2 and the like), killer T cells (e.g. natural killer T cells (NKT cells), cytotoxic T cells, and the like), naive T cells and the like.

As used herein, the terms “CD,” “CD antigen,” “Cluster of differentiation” refers to a designation assigned to leukocyte cell surface molecules. For example, “CD3” refers to but is not limited to a trimeric complex of γ, δ and ε chains which together with a ζζ homodimer or ζη heterodimer acts as a signal transducing unit for the T-cell receptor. Further, each CD molecule is identified by a given group of monoclonal antibodies. For example, “anti-CD3” and anti-CD3” and in certain situations CD3” refers to antibodies that bind to CD3 molecules. For another example, “anti-CD28” and anti-CD28” and in certain situations CD28” refers to antibodies that bind to CD28 molecules. As used herein, the term “CD4” refers to a cell surface glycoprotein, found on helper T-cells that are referred to as CD4 T cells. CD4 can function to recognize MHC class II molecules on antigen-presenting cells. As used herein, the term “CD8” refers to a cell surface glycoprotein, found on cytotoxic T-cells. CD8 can function to recognize MHC class I molecules on target cells.

As used herein, the terms “Th1 disease,” “Th1 disorder,” “type 1 autoimmune disease,” “type 1 autoimmune disorder” refers to a disorder wherein the immune response comprises a cellular immune response (e.g. T cell activation associated with IFN-gamma) that is more prominent than a humoral response (e.g. T cell activation associated with IL-4, B cell activation and antibody production). As used herein, the terms Th1 cytokine response and T1 cytokine response refers to an immune response whose most prominent feature comprises abundant CD4 helper T cell activation that is associated with increased levels of T1 cytokines (e.g. interferon-gamma, etc.) relative to these cytokine amounts in the absence of activation. A T1 cytokine response can also refer to the production of T1 cytokines from other white blood cells and nonwhite blood cells. A Th1 cytokine response can include abundant CD8 cytotoxic T lymphocyte activity including T1 cytokine production, referred to as Tc1. A Th1 response is typically promoted by CD4 “Th1” T-helper cells however a Th1 response can include CD8 Tc1 T cytotoxic cells.

As used herein, the terms “Type 1 cytokines,” “Th1 cytokines,” “Tc1 cytokines,” “T1 cytokines,” refers to cytokines that include but are not limited to IL-1, IL-2, IL-8, IL-12, IL-18, interferon-gamma, tumor necrosis factor-alpha (TNF-α), etc. Conversely, T1 cytokine production is not limited to T1 mediated diseases and disorders since increased levels of T1 cytokines might be found during certain stages of diseases and disorders classified as T2. Examples of T1 disorders include but are not limited to multiple sclerosis, type 1 autoimmune thyroid disorders,

As used herein, the terms “type 1 diabetes mellitus” and “insulin dependent diabetes” refers to a disorder following an immune system mediated loss of insulin from autoimmune destruction of the insulin-producing cells of the pancreas.

As used herein, the term “multiple sclerosis” refers to a chronic neurologic disease of the central nervous system (CNS). MS is classified but not limited to a demyelinating and an axonal disease. Multiple Sclerosis is a disease in which the immune system targets nerve tissues of the central nervous system. Most commonly, damage to the central nervous system occurs intermittently, allowing a person to lead a fairly normal life. At the other extreme, the symptoms may become constant, resulting in a progressive disease with possible blindness, paralysis, and the like.

As used herein, the term “rheumatoid arthritis” refers to arthritic symptoms wherein the immune system is presumed to predominantly target the lining (synovium) that covers various joints thus causing pain, swelling, and stiffness of the joints. Although antibodies are believed to have a role, the damage to the synovial lining is believed to be caused through cell-mediated inflammation and type 1 cytokine damage.

As used herein, the term “type 1 autoimmune thyroid disorder” refers to thyroid disorders that include but are not limited to Hashimoto's thyroiditis. As used herein, the term Hashimoto's thyroiditis refers to symptoms in human patients of hypoactive thyroid function that can be but is not limited to immune system destruction of thyroid tissue (e.g. excess type 1 cytokine, killer T cells whose activation destroys thyroid cells and tissues, etc.). In certain patients, circulating T cells produce higher levels of type 1 cytokines when compared to healthy people.

As used herein, the terms “Th2 disease,” “Th2 disorder,” “type 2 autoimmune disease,” “type 2 autoimmune disorder” refers to a disease or disorder wherein the immune response comprises a humoral response (e.g. T cell activation associated with IL-4, B cell activation and antibody production) that is more prominent than acellular immune response (e.g. T cell activation associated with IFN-γ). As used herein, the terms “Th2 cytokine response” and “T2 cytokine response” refers to an immune response whose most prominent feature comprises abundant CD4 helper T cell activation that is associated with increased levels of T2 cytokines (e.g. IL-4, etc.) relative to these cytokine amounts in the absence of activation. A T2 cytokine response can also refer to the production of T2 cytokines from other white blood cells and nonwhite blood cells. A Th2 cytokine response can include abundant CD8 cytotoxic T lymphocyte activity including T2 cytokine production, referred to as Tc2 responses. A Th1 response is typically promoted by CD4 “Th1” T-helper cells however a Th2 response can include CD8 Tc2T cytotoxic cells.

As used herein, the terms “Type 2 cytokines,” “Th2 cytokines,” “Tc2 cytokines,” “T2 cytokines,” refers to cytokines that include but are not limited to IL-4, IL-5, IL-6, IL-10, IL-13, IL-15, etc. Conversely, T2 cytokine production is not limited to T2 mediated diseases and disorders since increased levels of T2 cytokines might be found during certain stages of diseases and disorders classified as T1.

As used herein, the term “type 2 autoimmune thyroid disorder” refers to thyroid disorders that include but are not limited to Grave's disease.

As used herein, the term “Grave's disease” refers to symptoms in human patients of hyperactive thyroid function that can be but is not limited to immune system stimulation of thyroid tissue (e.g. thyroid hormone receptor stimulating antibodies).

As used herein, the terms “interleukin,” “IL,” “lymphokine,” and “cytokine” refers to a molecule secreted by a cell and a tissue including but not limited to a white blood cell (e.g. leukocyte) and a cell in a tissue (e.g. keratinocyte). As used herein, the abbreviated term “IL” refers to a numbered molecule (e.g. IL-2). As used herein, “IL receptors” refers to a receptor for that particular molecule. For example, a receptor for IL-2 is referred to as an IL-2R. IL molecules are received by receptors on the same cell and on other cells. As used herein, the terms engagement stimulation of interleukin receptors of a cell causes that cell to respond in a variety of ways depending on factors that include but are not limited to its type, differentiation stage, activation stage and cellular context. These responses include but are not limited to proliferation, activation, production of cytokine (e.g. increased transcription, increased translation, increase processing, autocrine production, paracrine production and the like), release of cytokines (e.g. autocrine, paracrine, portion of cytokine), inhibitory cytokines.

As used herein, the term “contacting” cells with an agent or antibody refers to placing the agent or a antibody in a location that will allow it to touch the cell in order to produce “contacted” cells. The contacting may be accomplished using any suitable method. For example, in one embodiment, contacting is by adding the agent or a antibody to a tube of cells. Contacting may also be accomplished by adding the agent to a culture of the cells. It is not meant to limit how the agent or antibody contacts the cells. In one embodiment, contacting may be accomplished by administration of agent or antibody to an animal in vivo.

As used herein, the term “anti-Th1” refers to any agent that reduces the levels and or activities of type 1 cytokines. It is intended that the term be used in its broadest sense, and includes, but is not limited to, agents described herein, for example those which are produced naturally or synthetically.

As used herein, the terms “antigen,” “immunogen,” “antigenic,” “immunogenic,” “antigenically active,” and “immunologically active” refer to any substance that is capable of inducing a specific humoral or cell-mediated immune response. An immunogen generally contains at least one epitope. Immunogens are exemplified by, but not restricted to molecules which contain a peptide, polysaccharide, nucleic acid sequence, and/or lipid. Complexes of peptides with lipids, polysaccharides, or with nucleic acid sequences are also contemplated, including (without limitation) glycopeptide, lipopeptide, glycolipid, etc. These complexes are particularly useful immunogens where smaller molecules with few epitopes do not stimulate a satisfactory immune response by themselves.

As used herein, the term “antigen-presenting cell” and “APC” refers to a term most commonly used when referring to white blood cells that present processed antigenic peptide and MHC class I and/or II molecules to the T-cell receptor on lymphocytes, (e.g. macrophages, dendritic cells, B-cells and the like). However, other non-white blood cells can also be referred to as “antigen-presenting cells” since they present peptides within MHC class I and class II to T-cells and the like, e.g. as occurs with viral infected cells, cancer cells and the like.

As used herein, the terms “dendritic cell,” “DC,” and “professional antigen-presenting cells” can evoke an antigen response at least 10× greater in magnitude when compared to APCs under similar conditions (reviewed in Mellman et al. Trends Cell Biol. June 8;(6): 231-7, 1998).

As used herein, the term “cell” refers to a single cell as well as to a population of (i.e., more than one) cells. The population may be a pure population comprising one cell type. Alternatively, the population may comprise more than one cell type. In the present invention, there is no limit on the number of cell types that a cell population may comprise.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

As used herein, the term “mixed cell culture,” refers to a mixture of two or more types of cells. In some embodiments, the cells are cell lines that are not genetically engineered, while in other embodiments the cells are genetically engineered cell lines. In some embodiments the cells contain genetically engineered molecules. The present invention encompasses any combination of cell types suitable for the detection, identification, and/or quantitation of apoptosis in samples, including mixed cell cultures in which some of the cell types used are not genetically engineered, mixtures in which one or more of the cell types are genetically engineered and the remaining cell types are not genetically engineered, and mixtures in which all of the cell types are genetically engineered.

As used herein, the term “primary cell” is a cell which is directly obtained from a tissue (e.g. blood) or organ of an animal in the absence of culture. Typically, though not necessarily, a primary cell is capable of undergoing ten or fewer passages in vitro before senescence and/or cessation of proliferation. In contrast, a “cultured cell” is a cell which has been maintained and/or propagated in vitro for ten or more passages.

As used herein, the term “cultured cells” refer to cells which are capable of a greater number of passages in vitro before cessation of proliferation and/or senescence when compared to primary cells from the same source. Cultured cells include “cell lines” and “primary cultured cells.”

As used herein, the term “cell line,” refers to cells that are cultured in vitro, including primary cell lines, finite cell lines, continuous cell lines, and transformed cell lines. But does not require, that the cells be capable of an infinite number of passages in culture. Cell lines may be generated spontaneously or by transformation.

As used herein, the terms “primary cell culture,” and “primary culture,” refer to cell cultures that have been directly obtained from cells in vivo, such as from animal or insect tissue. These cultures may be derived from adults as well as fetal tissue.

As used herein, the terms “monolayer,” “monolayer culture,” and “monolayer cell culture,” refer to cells that have adhered to a substrate and grow as a layer that is one cell in thickness. Monolayers may be grown in any format, including but not limited to flasks, tubes, coverslips (e.g., shell vials), roller bottles, etc. Cells may also be grown attached to microcarriers, including but not limited to beads.

As used herein, the term “suspension” and “suspension culture” refers to cells that survive and proliferate without being attached to a substrate. Suspension cultures are typically produced using hematopoietic cells, transformed cell lines, and cells from malignant tumors.

As used herein, the terms “culture media,” and “cell culture media,” refers to media that are suitable to support the growth of cells in vitro (i.e., cell cultures). It is not intended that the term be limited to any particular culture medium. For example, it is intended that the definition encompass outgrowth as well as maintenance media. Indeed, it is intended that the term encompass any culture medium suitable for the growth of the cell cultures of interest.

As used herein the term, the term “in vitro” refers to an artificial environment and to processes or reactions that occur within an artificial environment. In vitro environments exemplified, but are not limited to, test tubes and cell cultures. The term “in vivo” refers to the natural environment (e.g., an animal or a cell) and to processes or reactions that occur within a natural environment.

As used herein, the term “proliferation” refers to an increase in cell number.

As used herein, the term “differentiation” refers to the maturation process cells undergo whereby they develop distinctive characteristics, and/or perform specific functions, and/or are less likely to divide.

As used herein, the terms “isolated,” “to isolate,” “isolation,” “purified,” “to purify,” “purification,” and grammatical equivalents thereof as used herein, refer to the reduction in the amount of at least one contaminant (such as protein and/or nucleic acid sequence) from a sample. Thus purification results in an “enrichment,” i.e., an increase in the amount of a desirable protein and/or nucleic acid sequence in the sample.

As used herein, the term “amino acid sequence” refers to an amino acid sequence of a naturally occurring or engineered protein molecule. “Amino acid sequence” and like terms, such as “polypeptide,” “peptide” or “protein” are not meant to limit the amino acid sequence to the complete, native amino acid sequence associated with the recited protein molecule.

As used herein, the term “receptor proteins” and “membrane receptor proteins” refers to membrane spanning proteins that bind a ligand (e.g., a microbial molecule; endotoxin, such as LPS, LTA; dsRNA, and the like).

As used herein, the term “ligand” refers to a molecule that binds to a second molecule. A particular molecule may be referred to as either, or both, a ligand and second molecule. Examples of second molecules include a receptor of the ligand, and an antibody that binds to the ligand.

As is known in the art, “protein phosphorylation” is a common regulatory mechanism used by cells to selectively modify proteins carrying regulatory signals from outside the cell to the cytoplasm and ultimately the nucleus. The proteins that execute these biochemical modifications are a group of enzymes known as protein kinases. They may further be defined by the substrate residue that they target for phosphorylation. One group of protein kinases is the tyrosine kinases (TKs), which selectively phosphorylate a target protein on its tyrosine residues. Some tyrosine kinases are membrane-bound receptors (RTKs), and, upon activation by a ligand, can autophosphorylate as well as modify substrates. The initiation of sequential phosphorylation by ligand stimulation is a paradigm that underlies the action of such effectors as, for example, LPS, LTA, Lethal Toxin (LT), and interferons such as Interferon-.beta. (IFN-.beta.). The receptors for these ligands are tyrosine kinases and provide the interface between the binding of a ligand (hormone, growth factor) to a target cell and the transmission of a signal into the cell by the activation of one or more biochemical pathways. Ligand binding to a receptor tyrosine kinase activates its intrinsic enzymatic activity (See, e.g., Ullrich and Schlessinger, Cell 61:203-212, 1990). Tyrosine kinases can also be cytoplasmic, non-receptor-type enzymes and act as a downstream component of a signal transduction pathway.

As used herein, the term “protein kinase” refers to a protein that catalyzes the addition of a phosphate group from a nucleoside triphosphate to an amino acid in a protein. Kinases comprise the largest known enzyme superfamily and vary widely in their target proteins. Kinases can be categorized as protein tyrosine kinases (PTKs), which phosphorylate tyrosine residues, and protein serine/threonine kinases (STKs), which phosphorylate serine and/or threonine residues and the like. Some kinases have dual specificity for both serine/threonine and tyrosine residues. The majority of kinases contain a conserved 250-300 amino acid catalytic domain. This domain can be further divided into 11 subdomains. N-terminal subdomains I-IV fold into a two-lobed structure that binds and orients the ATP donor molecule, and subdomain V spans the two lobes. C-terminal subdomains VI-XI bind the protein substrate and transfer the gamma phosphate from ATP to the hydroxyl group of a serine, threonine, or tyrosine residue. Each of the 11 subdomains contains specific catalytic residues or amino acid motifs characteristic of that subdomain. For example, subdomain I contains an 8-amino acid glycine-rich ATP binding consensus motif, subdomain II contains a critical lysine residue that contributes to maximal catalytic activity, and subdomains VI through IX comprise the highly conserved catalytic core. STKs and PTKs also contain distinct sequence motifs in subdomains VI and VIII, which may confer hydroxyamino acid specificity. Some STKs and PTKs possess structural characteristics of both families. In addition, kinases may also be classified by additional amino acid sequences, generally between 5 and 100 residues, which either flank or occur within the kinase domain.

Non-transmembrane PTKs form signaling complexes with the cytosolic domains of plasma membrane receptors. Receptors that signal through non-transmembrane PTKs include cytokine, hormone, and antigen-specific lymphocytic receptors. Many PTKs were first identified as oncogene products in cancer cells in which PTK activation was no longer subject to normal cellular controls. In fact, about one third of the known oncogenes encode PTKs. Furthermore, cellular transformation (oncogenesis) is often accompanied by increased tyrosine phosphorylation activity (See, e.g., Carbonneau, H. and Tonks, Annu. Rev. Cell Biol. 8:463-93, 1992). Regulation of PTK activity may therefore be an important strategy in controlling some types of cancer.

Examples of protein kinases include, but are not limited to, cAMP-dependent protein kinase, protein kinase C, and cyclin-dependent protein kinases (See, e.g., U.S. Pat. Nos. 6,034,228; 6,030,822; 6,030,788; 6,020,306; 6,013,455; 6,013,464; and 6,015,807, all of which are incorporated herein by reference).

As used herein, the term “protein phosphatase” refers to proteins that remove a phosphate group from a protein. Protein phosphatases are generally divided into two groups, receptor-type and non-receptor type (e.g. intracellular) proteins. An additional group includes dual specificity phosphatases. Most receptor-type protein tyrosine phosphatases contain two conserved catalytic domains, each of which encompasses a segment of 240 amino acid residues (See e.g., Saito et al. Cell Growth and Diff. 2:59, 1991). Receptor protein tyrosine phosphatases can be subclassified further based upon the amino acid sequence diversity of their extracellular domains (See e.g., Krueger et al. Proc. Natl. Acad. Sci. USA 89:7417-7421, 1992). Examples of protein phosphatases include, but are not limited to, human protein phosphatase (PROPHO), FIN13, cdc25 tyrosine phosphatase, protein tyrosine phosphatase (PTP) 20, PTP 1D, PTP-D1, PTP .lambda., PTP-S31 (See e.g., U.S. Pat. Nos. 5,853,997; 5,976,853; 5,294,538; 6,004,791; 5,589,375; 5,955,592; 5,958,719; and 5,952,212; all of which are incorporated herein by reference).

As used herein, the term “activating” when in reference to a biochemical response (such as kinase activity) and/or cellular response (such as cell proliferation) refers to increasing the biochemical and/or cellular response.

As used herein, the term “activated” when in reference to a cell, refers to a cell that has undergone a response that alters its physiology and shifts it towards making a biologically response and becoming biologically “active” hence “activated.” For example, a monocyte becomes activated to mature into a macrophage. For another example, a macrophage becomes activated upon contact with endotoxin (such as LPS) wherein the activated macrophage can produce an increased level and/or type of molecule associated with activation (e.g. iNOS, MMP-12 Metalloelastase and the like). In another example, an immature dendritic cell becomes activated to mature into a functional dendritic cell. An “activated” cell does not necessarily, although it may, undergo growth or proliferation. Typically, activation of macrophages and DCs, unlike lymphocytes such as T-cells, B-cells and the like, does not stimulate proliferation. Activation can also induce cell death such as in activation-induced cell death (AICD) of T cells. In one embodiment of the present invention, activation can lead towards apoptotic death.

As used herein, the terms “naturally occurring,” “wild-type” and “wt” as used herein when applied to a molecule or composition (such as nucleotide sequence, amino acid sequence, cell, apoptotic blebs, external phosphatidylserine, etc.), mean that the molecule or composition can be found in nature and has not been intentionally modified by man. For example, a naturally occurring polypeptide sequence refers to a polypeptide sequence that is present in an organism that can be isolated from a source in nature, wherein the polypeptide sequence has not been intentionally modified by man.

The terms “derived from” and “established from” when made in reference to any cell disclosed herein refer to a cell which has been obtained (e.g., isolated, purified, etc.) from the parent cell in issue using any manipulation, such as, without limitation, infection with virus, transfection with DNA sequences, treatment and/or mutagenesis using for example chemicals, radiation, etc., selection (such as by serial culture) of any cell that is contained in cultured parent cells. A derived cell can be selected from a mixed population by virtue of response to a growth factor, cytokine, selected progression of cytokine treatments, adhesiveness, lack of adhesiveness, sorting procedure, and the like.

As used herein, the term “biologically active,” refers to a molecule (e.g. peptide, nucleic acid sequence, carbohydrate molecule, organic or inorganic molecule, and the like) having structured, regulatory, and/or biochemical functions.

Unless defined otherwise in reference to the level of molecules and/or phenomena, the terms “reduce,” “inhibit,” “diminish,” “suppress,” “decrease,” and grammatical equivalents when in reference to the level of any molecule (e.g., cytokine protein, nucleic acid sequence, protein sequence, kinase protein, kinase activity, etc.), and/or molecular complex (e.g. signaling proteins, and the like), phenomenon (e.g., protein-protein interactions, catalytic activity, apoptosis, cell death, cell survival, cell proliferation, caspase cleavage, receptor dimerization, receptor complex formation, DNA fragmentation, molecule translocation, binding to a molecule, expression of a nucleic acid sequence, transcription of a nucleic acid sequence, enzyme activity, etc.) in a first sample relative to a second sample, mean that the quantity of molecule and/or phenomenon in the first sample is lower than in the second sample by any amount that is statistically significant using any art-accepted statistical method of analysis. In one embodiment, the reduction may be determined subjectively, for example when a patient refers to their subjective perception of disease symptoms, such as pain, difficulty in breathing, clarity of vision, nausea, tiredness, etc. In another embodiment, the quantity of a molecule and/or a phenomenon in the first sample is at least 10% lower than, at least 25% lower than, at least 50% lower than, at least 75% lower than, and/or at least 90% lower than the quantity of the same molecule and/or phenomenon in a second sample.

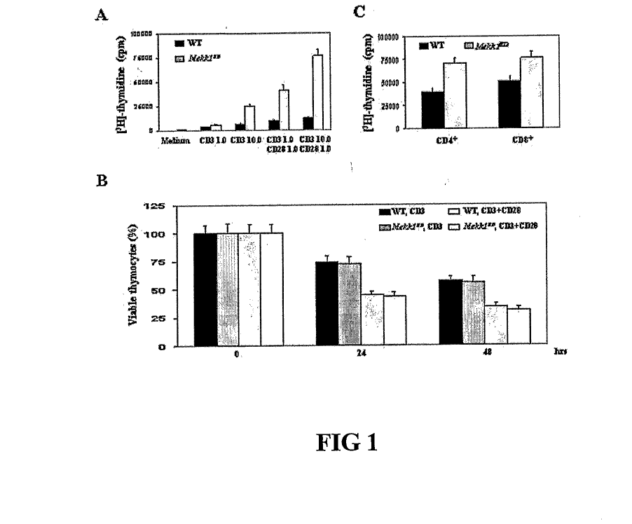

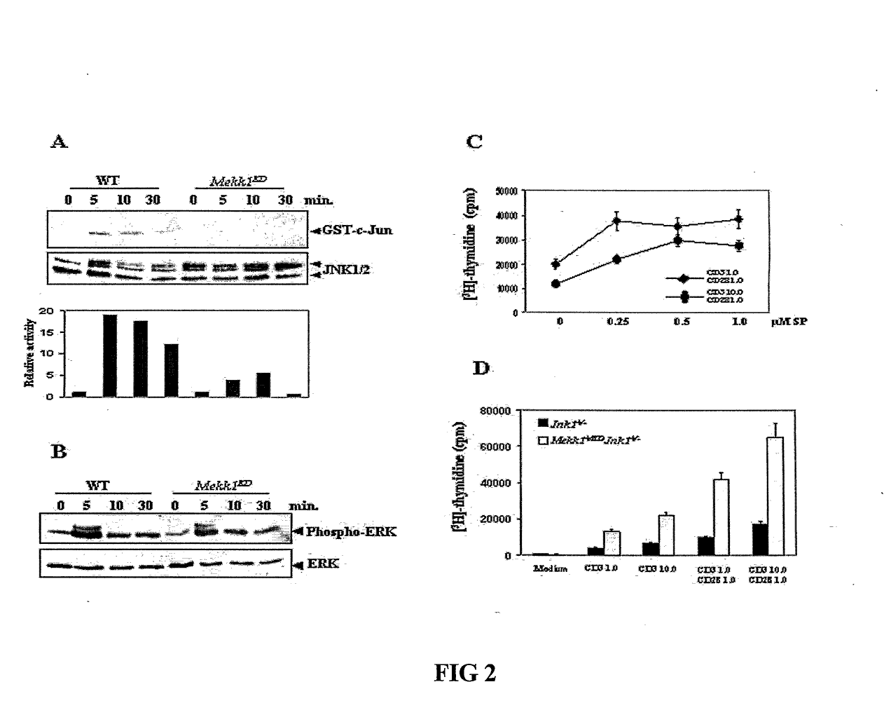

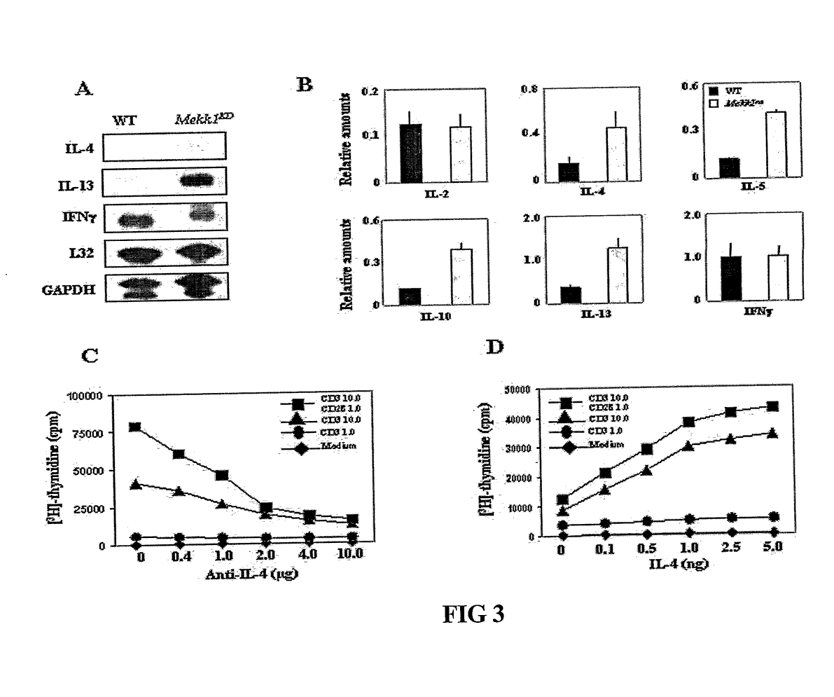

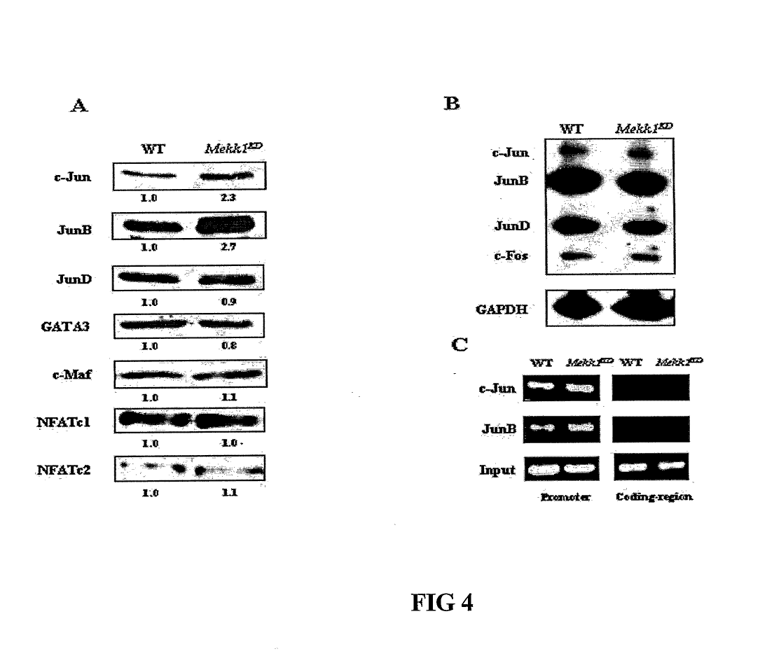

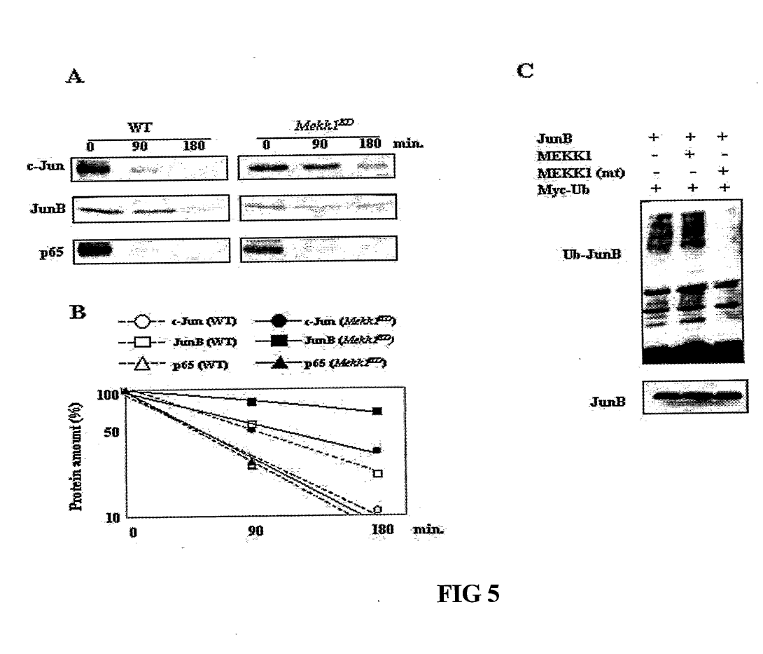

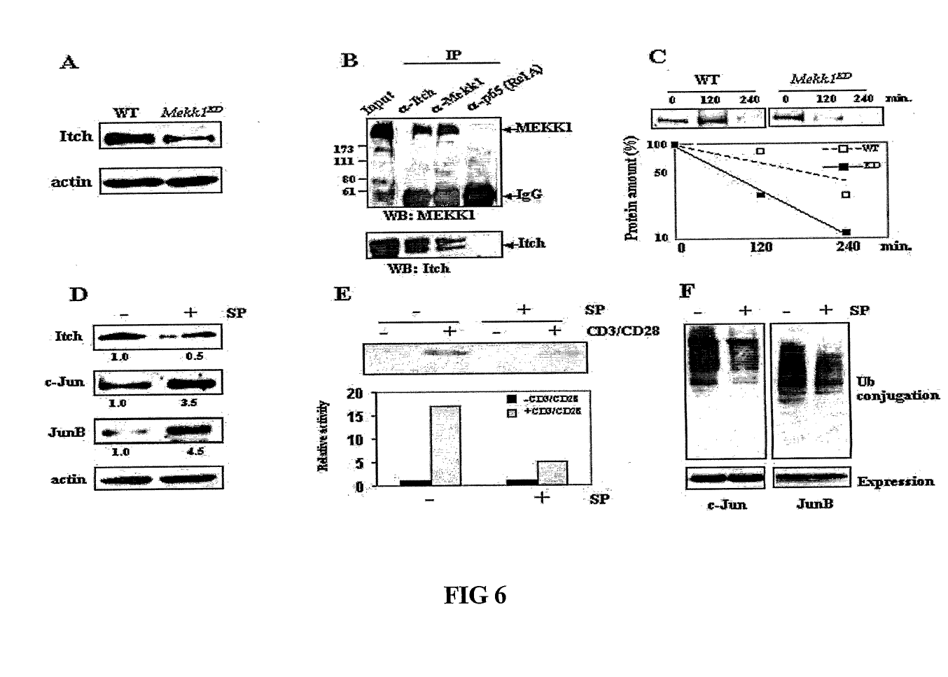

As exemplified herein, in one embodiment, the quantity of substance and/or phenomenon in the first sample is at least 5% lower than the quantity of the same substance and/or phenomenon in a second sample (e.g. FIG. 2B, FIG. 6C, and the like). In another embodiment, the quantity of the substance and/or phenomenon in the first sample is at least 25% lower than the quantity of the same substance and/or phenomenon in a second sample (e.g. FIG. 1B, FIG. 1C, and the like). In yet another embodiment, the quantity of the substance and/or phenomenon in the first sample is at least 50% lower than the quantity of the same substance and/or phenomenon in a second sample (e.g. FIG. 2B, FIG. 6D, and the like). In a further embodiment, the quantity of the substance and/or phenomenon in the first sample is at least 75% lower than the quantity of the same substance and/or phenomenon in a second sample (e.g. FIG. 5B, FIG. 6C, and the like). In yet another embodiment, the quantity of the substance and/or phenomenon in the first sample is at least 90% lower than the quantity of the same substance and/or phenomenon in a second sample (e.g. FIG. 3C, FIG. 3D and the like). In one embodiment, the reduction may be determined subjectively, for example when comparing mRNA levels (FIG. 2B, FIG. 4A and the like), etc.

As used herein, the term “apoptosis” refers to the process of non-necrotic cell death that takes place in metazoan animal cells following activation of an intrinsic cell suicide program. Apoptosis is a normal process in the proper development and homeostasis of metazoan animals and usually leads to cell death. Apoptosis is also triggered pathologically by microbial infections resulting in increasing susceptibility to apoptosis and/or outright death. Apoptosis involves sequential characteristic morphological and biochemical changes. One early marker of apoptosis is the flipping of plasma membrane phosphatidylserine, inside to outside, with cellular blebbing called “zeiosis,” of plasma membrane releasing vesicles containing cellular material including RNA and DNA as apoptotic bodies. During apoptosis, there is cell expansion followed by shrinkage through release of apoptotic bodies and lysis of the cell, nuclear collapse and fragmentation of the nuclear chromatin, at certain intranucleosomal sites, due to activation of endogenous nucleases. Apoptotic bodies are typically phagocytosed by other cells, in particular immunocytes such as monocytes, macrophages, immature dendritic cells and the like. One of skill in the art appreciates that reducing the ability to undergo apoptosis results in increased cell survival, without necessarily (although it may include) increasing cell proliferation. Accordingly, as used herein, the terms “reduce apoptosis” and “increase survival” are equivalent. Also, as used herein, the terms “increase apoptosis” and “reduced survival” are equivalent.

Apoptosis may be determined but not limited to; the assays described herein and include methods known in the art. For example, apoptosis may be determined by techniques for detecting DNA fragmentation, (for example any version of the Terminal deoxynucleotidyl transferase (TdT)-mediated dUTP Nick End-Labeling TUNEL technique originally developed by Gavrieli et al. J Cell Biol. November 1992;119(3):493-501, nuclear staining with nucleic acid dyes such as Hoechst 33342, Acridine Orange and the like, and detecting DNA “ladder” fragmentation patterns associated with apoptosis (e.g. DNA gels and the like)). In one embodiment, apoptosis is measured by TUNEL (for example, Park et al. Science 297, 2048-51, 2002). In one embodiment apoptosis is measured by observing DNA fragmentation in a ladder pattern (for example, Park et al. Science 297, 2048-51, 2002). Apoptosis may be determined by morphological measurements including but not limited to measuring live cells, early apoptotic cells, late apoptotic cells and cell death via apoptosis. For example, the cells' increased display of externally flipped phosphatidylserine, an early indicator of apoptosis, binds external Annexin-V. Thus Annexin-V attached to fluorescent molecules can be used to stain non permeablized cells and often further combined with vital dyes (e.g. trypan blue, propidium Iodide (PI), Ethidium Bromide (EtBr) and the like) allowing fluorescent activated cell sorting (FACS) analysis measuring of live, early apoptotic, late apoptotic and dead cells (Ozawa et al. J Exp Med. Feb. 15, 1999;189(4):711-8). An example of cell viability measurements is demonstrated in Example 2, and described in Example 1. Further, general live v. dead assays may also be employed, for example double staining with EtBr and Calcein AM for live microscopy determinations and FACS. Apoptosis may be determined by the presence of molecular fragments in apoptotic cells not present in live nonapoptotic cells. For example, caspase molecules such as Caspases-3, 6, 7, and 9 and the like, are cleaved during apoptotic processes, release of cytochrome c, PKR cleavage, and the like. Thus detecting the increased presence of predictable sizes of cleaved caspase subunits in apoptotic cells as compared to nonapoptotic cells indicate that cells are apoptotic. Furthermore, apoptosis may be monitored by changes in protein activity of molecules that decrease or increase cell survival and/or proliferation. For example, protein kinases and nuclear factors increase in activity during apoptosis and serve to either contribute to the apoptotic process or protect against apoptotic damage.

As used herein, the term “cellular response” refers to an increase or decrease of activity by a cell. For example, the “cellular response” may constitute but is not limited to apoptosis, death, DNA fragmentation, blebbing, proliferation, differentiation, adhesion, migration, DNA/RNA synthesis, gene transcription and translation, and/or cytokine secretion or cessation of such processes. A “cellular response” may comprise an increase or decrease of dephosphorylation, phosphorylation, calcium flux, target molecule cleavage, protein-protein interaction, nucleic acid-nucleic acid interaction, and/or protein/nucleic acid interaction and the like. As used herein, the term “target molecule cleavage” refers to the splitting of a molecule (for example in the process of apoptosis, cleavage of procaspases into fragments, cleavage of DNA into predicable sized fragments and the like). As used herein, the term “interaction” refers to the reciprocal action or influence of two or more molecules on each other.

As used herein, the term “phosphorylation” refers to the addition of phosphate groups. Protein phosphorylation is catalyzed by protein kinases which attach phosphate groups to hydroxyls of Ser, Thr and/or Tyr side chains. As used herein, the term “dephosphorylation” refers to the removal of a phosphate group. Protein dephosphorylation is catalyzed by protein phosphatases which remove phosphate groups from the side chains of Ser, Thr, and/or Tyr.

As used herein, the term “transgenic” when used in reference to a cell refers to a cell which contains a transgene, or whose genome has been altered by the introduction of a transgene. The term “transgenic” when used in reference to a tissue or to a plant refers to a tissue or plant, respectively, which comprises one or more cells that contain a transgene, or whose genome has been altered by the introduction of a transgene. Transgenic cells, tissues and plants may be produced by several methods including the introduction of a “transgene” comprising nucleic acid (usually DNA) into a target cell or integration of the transgene into a chromosome of a target cell by way of human intervention, such as by the methods described herein.

As used herein, the term “transgene” as used herein refers to any nucleic acid sequence which is introduced into the cell by experimental manipulations. A transgene may be an “endogenous DNA sequence” or a “heterologous DNA sequence” (i.e., “foreign DNA”). The term “endogenous DNA sequence” refers to a nucleotide sequence which is naturally found in the cell into which it is introduced so long as it does not contain some modification (e.g., a point mutation, the presence of a selectable marker gene, etc.) relative to the naturally occurring sequence. The term “heterologous DNA sequence” refers to a nucleotide sequence which is ligated to, or is manipulated to become ligated to, a nucleic acid sequence to which it is not ligated in nature, or to which it is ligated at a different location in nature. Heterologous DNA is not endogenous to the cell into which it is introduced, but has been obtained from another cell. Heterologous DNA also includes an endogenous DNA sequence which contains some modification. Generally, although not necessarily, heterologous DNA encodes RNA and proteins that are not normally produced by the cell into which it is expressed. Examples of heterologous DNA include reporter genes, transcriptional and translational regulatory sequences, selectable marker proteins (e.g., proteins which confer drug resistance), etc.

As used herein, the terms “agent,” “test agent,” “molecule,” “test molecule,” “compound,” and “test compound” as used interchangeably herein, refer to any type of molecule (for example, a peptide, nucleic acid, carbohydrate, lipid, organic molecule, and inorganic molecule, etc.) any combination molecule for example glycolipid, etc.) obtained from any source (for example, plant, animal, protist, and environmental source, etc.), or prepared by any method (for example, purification of naturally occurring molecules, chemical synthesis, and genetic engineering methods, etc.). Test agents are exemplified by, but not limited to individual and combinations of antibodies, nucleic acid sequences, and other agents as further described below.

In one embodiment, the term “test agent” refers to any chemical entity, pharmaceutical, drug, and the like that can be used to treat or prevent a disease, illness, sickness, or disorder of bodily function. Test agents comprise both known and potential therapeutic agents. A test agent can be determined to be therapeutic by screening using the screening methods of the present invention. A “known therapeutic agent” refers to a therapeutic agent that has been shown (e.g., through animal trials or prior experience with administration to humans) to be effective in such treatment or prevention. In other words, a known therapeutic agent is not limited to an agent efficacious in the treatment of disease (e.g., cancer). Agents are exemplified by, but not limited to, antibodies, nucleic acid sequences such as ribozyme sequences, and other agents as further described herein.

The test agents identified by and/or used in the invention's methods include any type of molecule (for example, a peptide, nucleic acid, carbohydrate, lipid, organic, and inorganic molecule, etc.) obtained from any source (for example, plant, animal, and environmental source, etc.), or prepared by any method (for example, purification of naturally occurring molecules, chemical synthesis, and genetic engineering methods, etc.).

Unless otherwise indicated, all numbers expressing quantities of ingredients, properties such as molecular weight, reaction conditions, and so forth as used herein, are to be understood as being modified in all instances by the term “about.” Accordingly, unless indicated to the contrary, the numerical parameters herein are approximations that may vary depending upon the desired properties sought to be obtained by the present invention. At the very least, and without limiting the application of the doctrine of equivalents to the scope of the claims, each numerical parameter should at least be construed in light of the number of reported significant digits and by applying ordinary rounding techniques. Notwithstanding that the numerical ranges and parameters describing the broad scope of the invention are approximations, the numerical values in the specific examples are reported as precisely as possible. Any numerical value, however, inherently contains standard deviations that necessarily result from the errors found in the numerical value's testing measurements.

The term “not” when preceding, and made in reference to, any particularly named molecule (e.g., nucleic acid sequence, protein sequence, apoptotic blebs, external phosphatidylserine, etc.), and/or phenomenon (e.g., apoptosis, cell death, cell survival, cell proliferation, caspase cleavage, receptor dimerization, receptor complex formation, DNA fragmentation, molecule translocation, binding to a molecule, expression of a nucleic acid sequence, transcription of a nucleic acid sequence, enzyme activity, etc.) means that primarily the particularly named molecule or phenomenon is excluded.

The term “altering” and grammatical equivalents as used herein in reference to the level of any molecule (e.g., nucleic acid sequence, protein sequence, apoptotic blebs, external phosphatidylserine, etc.), and/or phenomenon (e.g. apoptosis, cell death, cell survival, cell proliferation, caspase cleavage, receptor dimerization, receptor complex formation, DNA fragmentation, molecule translocation, binding to a molecule, expression of a nucleic acid sequence, transcription of a nucleic acid sequence, enzyme activity, etc.) refers to an increase and/or decrease in the quantity of the molecule and/or phenomenon, regardless of whether the quantity is determined objectively, and/or subjectively.

Unless defined otherwise in reference to the level of molecules and/or phenomena, the terms “increase,” “elevate,” “raise,” and grammatical equivalents when in reference to the level of any molecule (e.g., nucleic acid sequence, protein sequence, apoptotic blebs, external phosphatidylserine, etc.), and/or phenomenon (e.g., apoptosis, cell death, cell survival, cell proliferation, caspase cleavage, receptor dimerization, receptor complex formation, DNA fragmentation, molecule translocation, binding to a molecule, expression of a nucleic acid sequence, transcription of a nucleic acid sequence, enzyme activity, etc.) in a first sample relative to a second sample, mean that the quantity of the molecule and/or phenomenon in the first sample is higher than in the second sample by any amount that is statistically significant using any art-accepted statistical method of analysis. In one embodiment, the increase may be determined subjectively, for example when a patient refers to their subjective perception of disease symptoms, such as pain, difficulty in breathing, clarity of vision, nausea, tiredness, etc. In another embodiment, the quantity of the molecule and/or phenomenon in the first sample is at least 10% greater than, at least 25% greater than, at least 50% greater than, at least 75% greater than, and/or at least 90% greater than the quantity of the same molecule and/or phenomenon in a second sample.

Reference herein to any specifically named protein (such as mitogen-activated protein kinase/ERK kinase kinase 1, C-Jun N-terminal kinase 1, itch etc.) refers to any and all equivalent fragments, fusion proteins, and variants of the specifically named protein, having at least one of the biological activities (such as those disclosed herein and/or known in the art) of the specifically named protein, wherein the biological activity is detectable by any method.

The term “fragment” when in reference to a protein (such as mitogen-activated protein kinase/ERK kinase kinase 1, C-Jun N-terminal kinase 1, itch etc.) refers to a portion of that protein that may range in size from four (4) contiguous amino acid residues to the entire amino acid sequence minus one amino acid residue. Thus, a polypeptide sequence comprising “at least a portion of an amino acid sequence” comprises from four (4) contiguous amino acid residues of the amino acid sequence to the entire amino acid sequence.

The term “fusion protein” refers to two or more polypeptides that are operably linked. The term “operably linked” when in reference to the relationship between nucleic acid sequences and/or amino acid sequences refers to linking the sequences such that they perform their intended function. For example, operably linking a promoter sequence to a nucleotide sequence of interest refers to linking the promoter sequence and the nucleotide sequence of interest in a manner such that the promoter sequence is capable of directing the transcription of the nucleotide sequence of interest and/or the synthesis of a polypeptide encoded by the nucleotide sequence of interest. The term also refers to the linkage of amino acid sequences in such a manner so that a functional protein is produced.

The term “variant” of a protein (such as mitogen-activated protein kinase/ERK kinase kinase 1, C-Jun N-terminal kinase 1, itch, etc.) as used herein is defined as an amino acid sequence which differs by insertion, deletion, and/or conservative substitution of one or more amino acids from the protein of which it is a variant. The term “conservative substitution” of an amino acid refers to the replacement of that amino acid with another amino which has a similar hydrophobicity, polarity, and/or structure. For example, the following aliphatic amino acids with neutral side chains may be conservatively substituted one for the other: glycine, alanine, valine, leucine, isoleucine, serine, and threonine. Aromatic amino acids with neutral side chains which may be conservatively substituted one for the other include phenylalanine, tyrosine, and tryptophan. Cysteine and methionine are sulphur-containing amino acids, which may be conservatively substituted one for the other. Also, asparagine may be conservatively substituted for glutamine, and vice versa, since both amino acids are amides of dicarboxylic amino acids. In addition, aspartic acid (aspartate) may be conservatively substituted for glutamic acid (glutamate) as both are acidic, charged (hydrophilic) amino acids. Also, lysine, arginine, and histidine may be conservatively substituted one for the other since each is a basic, charged (hydrophilic) amino acid. Guidance in determining which and how many amino acid residues may be substituted, inserted or deleted without abolishing biological and/or immunological activity may be found using computer programs well known in the art, for example, DNAStar™ software. In one embodiment, the sequence of the variant has at least 95% identity, at least 90% identity, at least 85% identity, at least 80% identity, at least 75% identity, at least 70% identity, and/or at least 65% identity with the sequence of the protein in issue.

Reference herein to any specifically named nucleotide sequence (such as a sequence encoding mitogen-activated protein kinase/ERK kinase kinase 1, C-Jun N-terminal kinase 1, itch, etc.) includes within its scope any and all equivalent fragments, homologs, and sequences that hybridize under highly stringent and/or medium stringent conditions to the specifically named nucleotide sequence, and that have at least one of the biological activities (such as those disclosed herein and/or known in the art) of the specifically named nucleotide sequence, wherein the biological activity is detectable by any method.

The “fragment” or “portion” may range in size from an exemplary 5, 10, 20, 50, or 100 contiguous nucleotide residues to the entire nucleic acid sequence minus one nucleic acid residue. Thus, a nucleic acid sequence comprising “at least a portion of” a nucleotide sequence (such as sequences encoding mitogen-activated protein kinase/ERK kinase kinase 1, C-Jun N-terminal kinase 1, itch, etc.) comprises from five (5) contiguous nucleotide residues of the nucleotide sequence to the entire nucleotide sequence.

The term “homolog” of a specifically named nucleotide sequence refers to an oligonucleotide sequence, which exhibits greater than 50% identity to the specifically named nucleotide sequence (such as a sequence encoding mitogen-activated protein kinase/ERK kinase kinase 1, C-Jun N-terminal kinase 1, itch, etc). Alternatively, or in addition, a homolog of a specifically named nucleotide sequence is defined as an oligonucleotide sequence which has at least 95% identity, at least 90% identity, at least 85% identity, at least 80% identity, at least 75% identity, at least 70% identity, and/or at least 65% identity to nucleotide sequence in issue.

With respect to sequences that hybridize under stringent conditions to the specifically named nucleotide sequence (such as a sequence encoding mitogen-activated protein kinase/ERK kinase kinase 1, C-Jun N-terminal kinase 1, itch, etc.), high stringency conditions comprise conditions equivalent to binding or hybridization at 68° C. in a solution containing 5×SSPE, 1% SDS, 5× Denhardt's reagent and 100 μg/ml denatured salmon sperm DNA followed by washing in a solution containing 0.1×SSPE, and 0.1% SDS at 68° C. “Medium stringency conditions” when used in reference to nucleic acid hybridization comprise conditions equivalent to binding or hybridization at 42° C. in a solution of 5×SSPE (43.8 g/l NaCl, 6.9 g/l NaH₂PO₄—H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0×SSPE, 1.0% SDS at 42° C.

The term “equivalent” when made in reference to a hybridization condition as it relates to a hybridization condition of interest means that the hybridization condition and the hybridization condition of interest result in hybridization of nucleic acid sequences which have the same range of percent (%) homology. For example, if a hybridization condition of interest results in hybridization of a first nucleic acid sequence with other nucleic acid sequences that have from 85% to 95% homology to the first nucleic acid sequence, then another hybridization condition is said to be equivalent to the hybridization condition of interest if this other hybridization condition also results in hybridization of the first nucleic acid sequence with the other nucleic acid sequences that have from 85% to 95% homology to the first nucleic acid sequence.

As will be understood by those of skill in the art, it may be advantageous to produce a nucleotide sequence encoding a protein of interest, wherein the nucleotide sequence possesses non-naturally occurring codons. Therefore, in some embodiments, codons preferred by a particular prokaryotic or eukaryotic host (Murray et al., Nucl. Acids Res., 17 (1989)) are selected, for example, to increase the rate of expression or to produce recombinant RNA transcripts having desirable properties, such as a longer half-life, than transcripts produced from naturally occurring sequence.

A “composition” comprising a particular polynucleotide sequence (such as a sequence encoding mitogen-activated protein kinase/ERK kinase kinase 1, C-Jun N-terminal kinase 1, itch, etc.) and/or comprising a particular protein sequence (such as mitogen-activated protein kinase/ERK kinase kinase 1, C-Jun N-terminal kinase 1, itch, etc.) as used herein refers broadly to any composition containing the recited polynucleotide sequence (and/or its equivalent fragments, homologs, and sequences that hybridize under highly stringent and/or medium stringent conditions to the specifically named nucleotide sequence) and/or the recited protein sequence (and/or its equivalent fragments, fusion proteins, and variants), respectively. The composition may comprise an aqueous solution containing, for example, salts (e.g., NaCl), detergents (e.g., SDS), and other components (e.g., Denhardt's solution, dry milk, salmon sperm DNA, etc.).

The terms nucleotide sequence “comprising a particular nucleic acid sequence” and protein “comprising a particular amino acid sequence” and equivalents of these terms, refer to any nucleotide sequence of interest (such as a sequence encoding mitogen-activated protein kinase/ERK kinase kinase 1, C-Jun N-terminal kinase 1, itch, etc.) and to any protein of interest (such as mitogen-activated protein kinase/ERK kinase kinase 1, C-Jun N-terminal kinase 1, itch, etc.), respectively, that contain the particularly named nucleic acid sequence (and/or its equivalent fragments, homologs, and sequences that hybridize under highly stringent and/or medium stringent conditions to the specifically named nucleotide sequence) and the particularly named amino acid sequence (and/or its equivalent fragments, fusion proteins, and variants), respectively. The invention does not limit the source (e.g., cell type, tissue, animal, etc.), nature (e.g., synthetic, recombinant, purified from cell extract, etc.), and/or sequence of the nucleotide sequence of interest and/or protein of interest. In one embodiment, the nucleotide sequence of interest and protein of interest include coding sequences of structural genes (e.g., probe genes, reporter genes, selection marker genes, oncogenes, drug resistance genes, growth factors, etc.).

As used herein, the terms cascade pathway, signaling pathway, signal transduction pathway, MAP kinase pathway refers to an intracellular signaling system involving a network of proteins that include but are not limited to the MAP kinase cascades. An intracellular signaling system includes but is not limited to a response to extracellular stimuli that triggers a cascade by activating the first members (upstream activators) of a cascade. An general example of an upstream activator includes but is not limited to map kinase kinase kinases (MAPKKKs), these in turn induce phosphorylation of mitogen-activated protein kinase kinases (MAPKK) which in turn phosphorylate the mitogen-activated protein kinase; (MAPKs). The MAPKs then act on various downstream targets to affect gene expression. Examples of mammalian MAP kinase pathways including ERK (extracellular signal-regulated kinase) pathway, SAPK/JNK (stress-activated protein kinase/c-jun kinase) pathway, and p38 kinase pathway. It is not meant that these pathways are separate, in some embodiments, there is sharing of components among the pathways depending on many factors including but not limited to which stimulus originates activation of the cascade, the strength of stimulus, presence and strength of co-stimulatory signals, differentiation stage of the cell, activation stage of the cell, etc.

### DESCRIPTION OF THE INVENTION

The present invention relates to T helper 2 (Th2) cytokine production, and in particular, biasing the cytokine expression profile towards Th2 cytokine production through mitogen-activated protein kinase/ERK kinase kinase 1 (MEKK1), the screening of agents that increase Th2 cytokine production, and the treatment of T helper 1 (Th1) biased autoimmune diseases in vivo. In one embodiment, the present invention relates to agents including but not limited to reducing the activity of MEKK1, leading to increased levels of Th2 cytokine production.

While the present invention is not limited to any specific mechanism, the turnover of Jun proteins (like that of other transcription factors) is regulated through ubiquitin-dependent proteolysis. Usually, such processes are regulated by extracellular stimuli through phosphorylation of the target protein, which allows recognition by F box-containing E3 ubiquitin ligases. In the case of c-Jun and JunB, extracellular stimuli also modulate protein turnover by regulating the activity of an E3 ligase via its phosphorylation. Activation of the JNK mitogen-activated protein (MAP) kinase cascade after T cell stimulation accelerated degradation of c-Jun and JunB through phosphorylation-dependent activation of the E3 ligase Itch. This pathway modulates cytokine production by effector T cells.

T cell receptor (TCR) engagement results in activation of several transcription factors, including AP-1, leading to increased cytokine production. It was suggested that the intensity or duration of the activating stimulus bias the cytokine expression profile of T helper (Th) cells, but the underlying biochemical mechanism is unknown. Although TCR engagement can increase Jun/AP-1 transcriptional activity through JNK-mediated phosphorylation, the inventors found that it also promotes Jun protein turnover via a mechanism dependent on JNK and its upstream activator MEKK1, which associates with the E3 ubiquitin-ligase Itch. TCR activation stimulates Itch activity in a JNK-dependent manner and inhibition of either MEKK1 or JNK slows down the turnover of JunB and increases Th2 cytokine production and differentiation. This pathway may function to attenuate Th2 cytokine production following intense T cell stimulation.

Engagement of T cell receptor (TCR) along with co-stimulatory receptors, such as CD28, results in activation of several signaling pathways that stimulate the activity of various transcription factors involved in production of T cell cytokines (Crabtree et al. (1994) Annu Rev Biochem 63, 1045-1083; Weiss et al. (1994) Cell 76, 263-274). As used herein, the term “engagement” refers to when a receptor is triggered in such a way as to induce a biochemical response (e.g. activation of a signaling pathway and the like as demonstrated herein using anti-CD3 antibodies). As used herein, the term “T cell receptor,” “T-cell antigen receptor,” and “TCR” refers to T-cell antigen receptor typically consisting of either an α/β dimer or a γ/δ dimer associated with the CD3 molecular complex, in vivo. As used herein, the term “CD3 molecular complex” refers to the association of molecules that result in cellular biochemical responses.

These transcription factors include NF-AT and NF-κβ family members, as well as members of the AP-1 family (Crabtree et al. (1994) Annu Rev Biochem 63, 1045-1083; Nolan (1994) Cell 77, 795-798; Rao et al. (1997) Annu Rev Immunol 15, 707-747; Weiss et al. (1994) Cell 76, 263-274). In addition to cytokine gene induction, it was demonstrated that the intensity and duration of the signal generated by engagement of TCR and co-stimulatory receptors can modulate the differentiation of naïve T helper (Th) cells into the Th1 and Th2 effector subsets (Boyton et al. (2002) Trends Immunol 23, 526-529; Constant et al. (1995) J Exp Med 182, 1591-1596; Itoh et al. (1997) J Exp Med 186, 757-766; Kuchroo et al. (1995) Cell 80, 707-718; Lanzavecchia et al. (2000) Science 290, 92-97). While the mechanisms involved in the activation of T cell cytokine genes are relatively well understood (Murphy et al. (2000) Annu Rev Immunol 18, 451-494), the biochemical mechanisms by which TCR-generated signals can modulate the spectrum of cytokine gene expression are unknown. Nonetheless, it was observed that Th1 and Th2 cells exhibit fundamental differences in Ca2+ signaling (Gajewski et al. (1990) J Immunol 144, 4110-4120; Sloan-Lancaster et al. (1997) J Immunol 159, 1160-1168).

Some of the transcription factors required for Th cell polarization and differentiation were identified (Murphy et al. (2000) Annu Rev Immunol 18, 451-494). These include T-bet which is required for commitment to the Th1 phenotype (Szabo et al. (2000) Cell 100, 655-669) and GATA3 (Zheng et al. (1997) Cell 89, 587-596), c-Maf (Ho et al. (1996) Cell 85, 973-983), and JunB (Hartenstein et al. (2002) Embo J 21, 6321-6329; Li et al. (1999) Embo J 18, 420-432) for the Th2 phenotype. JunB is a member of the AP-1 family of transcription factors, which also includes c-Jun and JunD, as well as c-Fos, FosB, Fra1 and Fra2 and several other proteins (Angel et al. (1991) Biochim Biophys Acta 1072, 129-157). AP-1 activity is subject to complex regulation, in which members of the mitogen activated protein kinase (MAPK) family play an important role both by regulating the expression of Jun and Fos genes, as well as by modulating the transcriptional activity of their protein products through direct phosphorylation (Karin (1995) J Biol Chem 270, 16483-16486). Amongst the key regulators of AP-1 activity are the Jun N-terminal kinases (JNKs), which in addition to the Jun proteins (Kallunki et al. (1996) Cell 87, 929-939), also phosphorylate other transcription factors (Minden et al. (1997) Biochim Biophys Acta 1333, F85-104). Importantly, in T cells the JNKs are primary members of the MAPK family whose activation in response to TCR engagement is strongly potentiated by occupancy of the co-stimulatory receptor CD28 (Su et al. (1994) Cell 77, 727-736). These findings suggested that JNK may be involved in the interpretation of co-stimulatory signals or the intensity of T cell activation. TCR-mediated JNK activation depends on Ca2+ signaling (Werlen et al. (1998) Embo J 17, 3101-3111).

As used herein, co-stimulatory signals, co-stimulatory molecules, co-stimulatory stimulation, “stimulatory signal,” and co-stimulation refers to interactions between cells of the immune system wherein a costimulatory “second messenger” molecule controls the cell-to-cell transmission of a signal that initiates an immune response. For example, CD28 is a receptor referred to as a co-stimulatory molecule in that triggering of CD28 in addition to CD3 enhances the CD3 response. It is not meant that co-stimulation be limited to receptors in that co-stimulation can be accomplished by the use of antibodies (e.g. Example 2).

In addition to direct binding to several cytokine gene promoters, such as that of IL-4 (Li et al. (1999) Embo J 18, 420-432; Rooney et al. (1995) Immunity 2, 473-483), AP-1 proteins are required for cooperative binding of NF-AT proteins to low affinity sites (Rao et al. (1997) Annu Rev Immunol 15, 707-747; Rooney et al. (1995) Immunity 2, 473-483). While at least three Jun proteins are expressed in T cells, it was found that differentiation towards the Th2 phenotype is accompanied by up-regulation of JunB, whose ectopic expression can polarize naïve Th cells towards the Th2 phenotype (Li et al. (1999) Embo J 18, 420-432). Gene disruption experiments confirmed the critical role of JunB in expression of Th2 cytokines (Hartenstein et al. (2002) Embo J 21, 6321-6329), which include IL-4, IL-5, IL-10 and IL-13 (Murphy et al. (2000) Annu Rev Immunol 18, 451-494; Paul et al. (1994) Cell 76, 241-251). JunB can directly activate the IL-4 promoter in cooperation with c-Maf and this activity is strongly potentiated by JNK-mediated JunB phosphorylation (Li et al. (1999) Embo J 18, 420-432). Despite the ability of JNK to enhance JunB (and c-Jun) transcriptional activity, the loss of both JNK1 and JNK2 expression strongly enhances, rather than attenuates, the generation of Th2 effector cells (Dong et al. (2000) Nature 405, 91-94). These results suggest that the JNKs may have a complex and rather enigmatic role in regulation of JunB or other transcription factors required for expression of Th2 cytokines.

As used herein, the terms “JNK1,” “c-Jun N-terminal kinase 1,” “Mitogen-activated protein kinase 8,” “Stress-activated protein kinase JNK1,” and “JNK-46” refers to the protein encoded by genes including but not limited to JNK1 or MAPK8 or PRKM8.

As used herein, the terms “JNK2,” “c-Jun N-terminal kinase 2,” “Mitogen-activated protein kinase 9,” “Stress-activated protein kinase JNK2,” and “JNK-55” refers to the protein encoded by genes including but not limited to JNK2 or MAPK9 or PRKM9.

Another protein that is involved in the regulation of Jun protein activity or expression in T cells is the “E3 ubiquitin ligase itch” also called “Itch” (Fang et al. (2002) Nat Immunol 3, 281-287).

As used herein, the terms “Itchy E3 ubiquitin protein ligase,” “itch E3 ubiquitin protein ligase,” “E3 ubiquitin protein ligase itch,” “Itchy homolog E3 ubiquitin protein ligase,” “Itch,” “itchy,” “Atrophin-1-interacting protein 4,” “AIP4,” “NFE2-associated polypeptide 1,” and “NAPP1” refers to mouse and human proteins whose gene names include but are not limited to ITCH.

Itchy mutant mice, which express an inactive form of Itch, exhibit increased expression of Th2 cytokines as well as elevated levels of c-Jun and JunB in their T cells (Fang et al. (2002) Nat Immunol 3, 281-287). Itch is a member of the HECT domain group of E3 ubiquitin-protein ligases (Qiu et al. (2000) J Biol Chem 275, 35734-35737). These proteins recognize their substrates via a WW domain, which unlike the F boxes used by members of the SCF group of ubiquitin-protein ligases, do not recognize phospho-epitopes (Ciechanover et al. (2000) Bioessays 22, 442-451; Joazeiro et al. (2000) Science 289, 2061-2062). Yet, Itch was shown to interact with both JunB and c-Jun and stimulate their polyubiquitination in an in vitro system (Fang et al. (2002) Nat Immunol 3, 281-287).

Analysis was made on the role of a JNK pathway in regulation of AP-1 and Jun activity in T cells and a role of the MAPK kinase (MAP2K) kinase (MAP3K) MEKK1 in TCR-mediated JNK activation and gene expression.

As used herein, the terms “MEKK1,” “Mitogen-activated protein kinase kinase kinase 1,” “MAP3K,” “MAPK/ERK kinase kinase 1,” “MEK kinase 1,” and mitogen-activated protein kinase/ERK kinase kinase 1” refers to mouse and human proteins whose gene names include but are not limited to “MEKK1,” “MAP3K,” “MAPKKK1” “MEKK” and the like.

As used herein, the terms “MEKK4,” “Mitogen-activated protein kinase kinase kinase 4,” “MAPK/ERK kinase kinase 4,” “MEK kinase 4,” “MEKK 4” refers to mouse and human proteins whose gene names include but are not limited to “MAP3K4” or “MAPKKK4” or “MEKK4” or “MTK1” or “KIAA0213.”

As used herein, the terms “MEKK7,” “Dual specificity mitogen-activated protein kinase kinase 7,” “MAP kinase kinase 7,” “MAPKK7,” “MAPK/ERK kinase 7,” “JNK activating kinase 2,” “c-Jun N-terminal kinase kinase 2,” “JNK kinase 2,” “JNKK 2,” “MKK7” refers to mouse and human proteins whose gene names include but are not limited to “MAP2K7” or “MKK7” or “MAP2K7” or “PRKMK7” or “JNKK2” or “MKK7.”

MEKK1 is one of the most potent activators of the JNK cascade identified (Minden et al. (1997) Biochim Biophys Acta 1333, F85-104). Here the inventors show that MEKK1 is the major activator of the JNK cascade in response to TCR and CD28 co-ligation. Interestingly, disruption of MEKK1 catalytic activity or JNK inhibition results in marked overexpression of Th2 cytokines while having no effect on Th1 cytokines. Furthermore, like the inactivation of Itch, the loss of MEKK1 catalytic activity results in stabilization of c-Jun and JunB. The inventors show that MEKK1 physically interacts with Itch and can regulate its stability and enhance the polyubiquitination of JunB. Based on these results the inventors suggest that TCR and CD28 co-ligation results in activation of the MEKK1 to JNK cascade, which has two opposing effects on JunB and c-Jun. While enhancing their transcriptional activity through direct phosphorylation, it also leads to their increased turnover and eventual degradation. The latter effect could be the biochemical mechanism through which output from the TCR and co-stimulatory receptors modulates Th cell differentiation.

### Results

### TCR Hyperresponsiveness in Mekk1^{KD} Mutant T Cells

Although the role of JNK1 and JNK2, as well as the upstream kinase MKK7, in both thymocyte and peripheral T cell activation has been examined, some of the results were rather controversial (Dong et al. (2000) Nature 405, 91-94; Sabapathy et al. (2001) J Exp Med 193, 317-328; Sasaki et al. (2001) J Exp Med 194, 757-768) and as discussed above inconsistent with results obtained for the JNK substrate JunB (Hartenstein et al. (2002) Embo J 21, 6321-6329; Li et al. (1999) Embo J 18, 420-432). The inventors were interested in understanding whether MEKK1 is the MAP3K responsible for JNK activation in response to TCR engagement and if so examine its function in T cell biology. The loss of MEKK1 catalytic activity (Xia et al. (2000) Proc Natl Acad Sci USA 97, 5243-5248) had no effect on thymocyte numbers and differentiation into the CD4 and CD8 double and single positive classes (unpublished results). However, MEKK1 kinase-deficient (Mekk1^{KD}) thymocytes were hyperresponsive to TCR engagement with anti-CD3 antibody and this hyperresponsiveness was strongly augmented by ligation of the co-stimulatory CD28 receptor (FIG. 1A). Proliferation in response to PMA and ionomycin, stimuli that bypass the TCR and CD28, was not affected. The increased proliferative response of Mekk1^{KD} thymocytes was not due to decreased activation-induced death (FIG. 1B). In fact, activation-induced death was slightly elevated in mutant cells. In addition, the expression levels of several cell death regulators were unchanged in mutant thymocytes. Similar, but not as pronounced, hyperresponsiveness was observed in peripheral Mekk1^{KD} T cells (FIG. 1C), whereas the rate of cell death was comparable between WT and Mekk1^{KD} splenic T cells.

Decreased JNK Activation in Mekk1^{KD} Mutant Thymocytes Underlies their Hyperresponsiveness

The inventors next examined the effect of the Mekk1^{KD} mutation on activation of JNK and other MAPKs in response to TCR and CD28 engagement. Loss of MEKK1 catalytic activity resulted in a considerable decrease in JNK activation following stimulation of thymocytes with anti-CD3+anti-CD28 (FIG. 2A). By contrast, ERK activation was not affected (FIG. 2B). p38 MAPK was not activated by these stimuli in either WT or Mekk1^{KD} thymocytes. To determine whether loss of JNK activity causes TCR hyperresponsiveness, the inventors used the small molecule JNK inhibitor SP600125 (Bennett et al., 2001). Stimulation of WT thymocytes with different concentrations of anti-CD3+anti-CD28 in the presence of increasing amounts of SP600125, which inhibited JNK activity (see below), resulted in at least 2-fold increase in [3H] thymidine incorporation (FIG. 2C). To confirm that the effect of MEKK1 on thymocyte proliferation is mediated through JNK, the inventors bred Jnk1^{-/-} mice with Mekk1^{KD} mice. Interestingly, Mekk1^{+/KD}Jnk1^{±} mice showed open eyelids at birth, the same phenotype exhibited by Mekk1^{KD/KD} mice, while both Mekk1^{+/KD} and Jnk1^{-/-} mice were born with closed eyes. Importantly, Mekk1^{±}Jnk1^{±} thymocytes exhibited a pronounced hyperresponsiveness to TCR and CD28 engagement in comparison to either Jnk1^{±} (FIG. 2D) or Mekk1^{+/KD} thymocytes.

### Increased Th2 Cytokine Production by Mekk1^{KD} Thymocytes

To determine the mechanism by which reduced JNK and MEKK1 activity leads to thymocyte and T cell hyperresponsiveness, the inventors compared the pattern of TCR and downstream effector protein tyrosine phosphorylation between WT and mutant cells and found negligible obvious differences. We also compared the levels of various cyclins, cyclin-dependent kinases (CDKs) and CDK inhibitors, including p16, between WT and mutant thymocytes and found negligible major differences in their expression. Next the inventors considered the role of cytokine gene expression in the hyperresponsive phenotype of Mekk1^{KD} T cells, as cytokines play crucial roles in regulating T cell activation and differentiation (Murphy et al. (2000) Annu Rev Immunol 18, 451-494; Paul et al. (1994) Cell 76,241-251; Smith et al. (1979) Ann NY Acad Sci 332,423-432). Indeed, Mekk1^{KD} thymocytes were found to express elevated levels of IL-4 and IL-13 mRNAs but close to normal levels of interferon-gamma (IFN-γ) mRNA after stimulation with anti-CD3+anti-CD28 (FIG. 3A). Unstimulated thymocytes expressed primarily background levels of these mRNAs. In addition to ribonuclease (RNase) protection analysis, the inventors used a more sensitive real-time PCR assay to quantitate the levels of these and other mRNAs in WT and Mekk1^{KD} thymocytes after 48 hrs stimulation with anti-CD3+anti-CD28. While expression of IL-2 or IFN-g mRNAs was not affected, Mekk1^{KD} cells expressed up to 5-fold more IL-4, IL-5, IL-10 and IL-13 mRNAs (FIG. 3B). Upregulation of IL-4 mRNA was also observed by microarray analysis. The cytokine mRNAs that are overexpressed in Mekk1^{KD} thymocytes are characteristic of the Th2 effector cell type (Murphy et al. (2000) Annu Rev Immunol 18, 451-494; Paul et al. (1994) Cell 76, 241-251). Indeed, when induced to differentiate, naïve Mekk1^{KD} Th cells exhibited a marked bias towards the Th2 type (T. Naumaner, unpublished data).

To determine whether increased IL-4 production was responsible for the hyperproliferation of Mekk1^{KD} thymocytes, the inventors examined the effect of a neutralizing anti-IL-4 antibody. Incubation with anti-IL-4, but not control anti-IgG, markedly reduced the proliferation of Mekk1^{KD} thymocytes elicited by anti-CD3 in the absence or presence of anti-CD28 (FIG. 3C). In addition, addition of exogenous IL-4 to WT thymocytes strongly potentiated their proliferative response to the same stimuli (FIG. 3D).

Elevated c-Jun and JunB Expression in Mekk1^{KD} Thymocytes

The molecular regulation of IL-4, IL-5, and IL-13 gene expression was recently reviewed (Murphy et al. (2000) Annu Rev Immunol 18, 451-494). Multiple transcription factors, including NF-AT, GATA-3, c-Maf, JunB, are involved in IL-4 gene induction (Murphy et al., 2000; Paul and Seder, 1994). JunB (Li et al., 1999), as well as its relative c-Jun (Hibi et al. (1993) Genes Dev 7, 2135-2148; Kallunki et al. (1996) Cell 87, 929-939), are targets for JNK-mediated phosphorylation. Curiously, the inventors found that both c-Jun and JunB were elevated in activated Mekk1^{KD} thymocytes, while expression of JunD, another family member, was unchanged (FIG. 4A). Mekk1^{KD} cells, also expressed normal levels of GATA-3, c-Maf, NF-ATc1 and NF-ATc2. Surprisingly, despite the change in protein levels, the levels of c-Jun and JunB mRNAs were not different between WT and mutant thymocytes (FIG. 4B).

To confirm that JunB, and possibly c-Jun, functions as a transcriptional regulators of the IL-4 gene, the inventors performed chromatin immunoprecipitation (ChIP) experiments. Recruitment of both c-Jun and JunB to the proximal IL-4 promoter region was detected in both WT and Mekk1^{KD} thymocytes activated with anti-CD3+anti-CD28 (FIG. 4C). Neither protein was recruited to the IL-4 coding region. Control experiments with anti-p65 (RelA) antibodies did not result in precipitation of the proximal IL-4 promoter region, consistent with the absence of NF-κβ binding sites (Murphy et al. (2000) Annu Rev Immunol 18, 451-494). In multiple repeats of this experiment the inventors observed that the IL-4 promoter signal was modestly, but reproductively, more intense when the ChIP was performed on mutant cells.

MEKK1 Promotes Ubiquitin-Dependent Degradation of c-Jun and JunB

A possible mechanism through which MEKK1 and JNK can modulate c-Jun and JunB levels is by promoting their turnover. To examine this point, the inventors conducted pulse-chase experiments. In WT thymocytes, newly synthesized c-Jun and JunB proteins were degraded with half-lives (t½) of 59 and 78 min, respectively (FIG. 5A, B). Both proteins were significantly more stable in Mekk1^{KD} thymocytes, where their t½ was extended to 109 and 280 min, respectively. By contrast, the turnover of RelA (p65) was unaltered between WT and Mekk1^{KD} cells.

Degradation of many short-lived transcription factors is controlled by ubiquitin-dependent proteolysis (Hershko et al. (1998) Annu Rev Biochem 67, 425-479). The inventors examined whether MEKK1 is involved in c-Jun or JunB ubiquitination. Due to difficulties in detecting endogenously ubiquitinated proteins, the inventors used transiently transfected cells. 293T cells were transfected with plasmids encoding Myc-tagged ubiquitin, hemagglutinin (HA)-tagged c-Jun or JunB, and either WT or kinase domain-truncated MEKK1. Cell lysates were immunoprecipitated with anti-HA, and ubiquitination of c-Jun and JunB was monitored by immunoblotting with anti-Myc. Ectopic expression of WT MEKK1 promoted Ub conjugation to both c-Jun and JunB (FIG. 5C,). By contrast, overexpression of the MEKK1 mutant resulted in weaker Jun polyubiquitination. This information suggest that MEKK1 promotes c-Jun and JunB turnover by enhancing the extent of their ubiquitination.

The MEKK1 I/JNK Cascade Regulates Itch Turnover and Expression, as well as Jun Ubiquitination and Cytokine Production

An important role in protein ubiquitination is played by the E3 ubiquitin protein ligases, which are responsible for substrate recognition (Hershko et al. (1998) Annu Rev Biochem 67,425-479). Elevated expression of c-Jun and JunB and increased Th2 cytokine production were recently described in T cells from Itchy mutant mice that express an inactive form of the Itch E3 ubiquitin protein ligase. These similarities raised the possibility that MEKK1-directed JunB (or c-Jun) turnover is Itch-dependent and that the MEKK1/JNK cascade controls Itch activity or expression. The inventors found that Mekk1^{KD} thymocytes expressed lower levels of Itch before and after activation (FIG. 6A Although decreased Itch expression was also observed in Mekk1^{KD} splenic T cells and B cells, Itch was expressed at normal levels in the heart and liver of mutant mice. Importantly, Itch and MEKK1 exhibited very efficient and nearly quantitative interaction in thymocytes and splenic T cells (FIG. 6B).

Despite reduced Itch protein, the level of Itch mRNA was identical between WT and Mekk1^{KD} lymphocytes. To investigate whether MEKK1 controls Itch protein turnover, the inventors conducted pulse-chase experiments. In Mekk1^{KD} cells, newly synthesized Itch was degraded with a t½ of 77 min, whereas in WT cells its t½ was 136 min (FIG. 6C). Thus the absence of MEKK1 kinase activity results in destabilization of Itch, while it stabilizes c-Jun and JunB.

To test whether this effect of MEKK1 is mediated through JNK, the inventors used the JNK inhibitor. Incubation of activated WT thymocytes with SP600125 resulted in a 4-fold increase in c-Jun and JunB expression and a 2-fold decrease in Itch expression after 24 hrs (FIG. 6D). Negligible such effects were observed upon incubation with the p38 inhibitor SB202190. The effect of SP600125 on Jun and Itch expression correlated with its effect of JNK activation (FIG. 6E). The inventors also found that treatment with the JNK inhibitor reduced the extent of c-Jun and JunB polyubiquitination in transfected cells (FIG. 6F). In addition to the effects on Jun and Itch expression, treatment of activated thymocytes with the JNK inhibitor resulted in increased IL-4 mRNA expression.

### Discussion

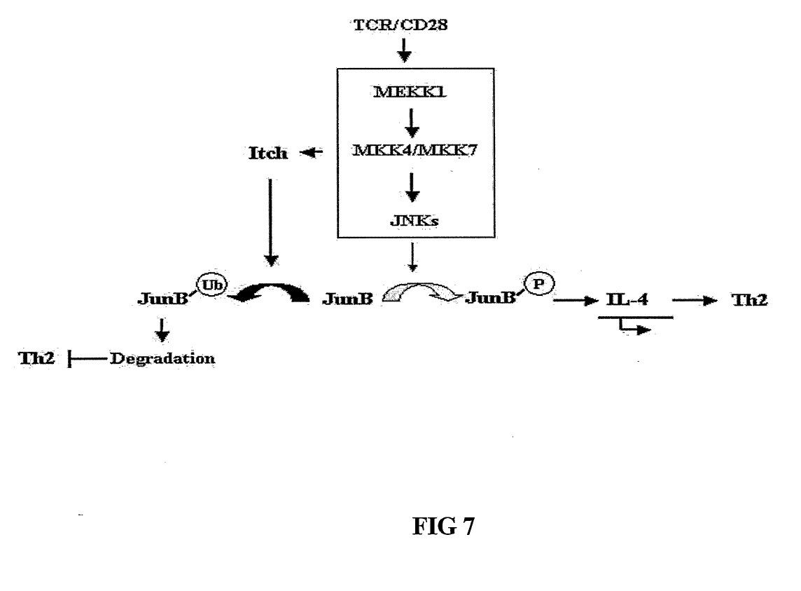

The results described above chart a novel-signaling pathway that leads to down-regulation of Jun/AP-1 activity in T cells in response to TCR and CD28 engagement by accelerating the turnover of JunB and c-Jun (FIG. 7). Previous studies have indicated that AP-1 proteins play an important role in induction of T cell cytokine genes both by direct binding to AP-1 sites in their promoters and by enabling NF-AT proteins to recognize low affinity sites (Rao et al. (1997) Annu Rev Immunol 15, 707-747; Rooney et al. (1995) Immunity 2, 473-483). Furthermore, the transcriptional activities of both c-Jun and JunB were shown to be enhanced by JNK-mediated phosphorylation in T cells (Li et al. (1999) Embo J 18,420-432; Su et al. (1994) Cell 77, 727-736). These findings led to the expectation that c-Jun and JunB and the pathway that regulates their activity, namely the JNK pathway, play a positive role in expression of T cell cytokine genes. This expectation was partially borne out for JunB, whose transgenic overexpression was shown to increase the expression of Th2 cytokine genes (Li et al. (1999) Embo J 18, 420-432), while its ablation severely attenuated their induction (Hartenstein et al. (2002) Embo J 21, 6321-6329). Interestingly, however, the manipulation of JunB levels did not alter the expression of Th1 cytokines, such as IFN-g, or IL-2, despite the presence of AP-1 sites in their promoters (Penix et al. (1996) J Biol Chem 271, 31964-31972; Serfling et al. (1989) Embo J 8, 465-473). Curiously, however, the ablation of both JNK1 and JNK2, the protein kinases responsible for enhanced JunB activity (Li et al. (1999) Embo J 18, 420-432), was found to dramatically enhance, rather than inhibit, the production of Th2 cytokines (Dong et al. (2000) Nature 405, 91-94). Similar results were obtained by ablation of MKK7 (JNKK2), the MAP2K responsible for JNK activation in T cells (Dong et al. (2000) Nature 405, 91-94), although one study attributed the hyperproliferation of MKK7-deficient lymphocytes to decreased p16 expression (Sasaki et al. (2001) J Exp Med 194, 757-768), an affect not seen in Mekk1^{KD} T cells. However, none of these studies provided a mechanistic explanation for the negative effect of the JNK pathway on Th2 cytokine expression, neither were the targets for this inhibitory effect identified. The inventors now show that elimination of MEKK1 catalytic activity in thymocytes and mature T cells results in eventually the same effect on cell proliferation and Th2 cytokine production as produced by elimination of JNK1/2 or MKK7 activities. The inventors provide both biochemical and genetic evidence that the inhibitory effect of MEKK1 on T cell proliferation and Th2 cytokine gene expression is mediated via JNK, as already suggested by the analysis of JNK1/2-and MKK7-deficient T cells (Dong et al. (2000) Nature 405, 91-94). Most importantly, the inventors have identified the biochemical pathway through which the MEKK1-dependent JNK cascade exerts this negative activity. The inventors show that thymocytes lacking MEKK1 catalytic activity accumulate both c-Jun and JunB, while they continue to express normal levels of other transcription factors involved in Th2 cytokine expression or JunD. The accumulation of c-Jun and JunB is shown to be due to reduced JNK activity as well as their decreased degradation. The effect of the MEKK1/JNK cascade on Jun protein turnover and cytokine production is essentially identical to that of the E3 ubiquitin ligase Itch. Previous studies revealed that Itchy mice, expressing an inactive form of Itch, overproduce Th2 cytokines and accumulate c-Jun and JunB in their T cells, while expressing normal levels of JunD or other transcription factors (Fang et al. (2002) Nat Immunol 3, 281-287). Most interestingly, the inventors identify a very efficient and nearly quantitative interaction between MEKK1 and Itch and show that reduced MEKK1 or JNK activity results in accelerated Itch turnover, while slowing the turnover of c-Jun and JunB.

The inventors results suggest that the primary function of the MEKK1/JNK cascade in T cells undergoing robust activation brought about by strong co-stimulation of both TCR and CD28 is to enhance the Itch-dependent degradation of important regulatory proteins, such as JunB. As JunB is required for Th2, but not Th1, cytokine production (Hartenstein et al. (2002) Embo J 21, 6321-6329), the overall effect of this pathway is to attenuate the production of IL-4, the critical Th2 cytokine (Paul et al. (1994) Cell 76, 241-251), and thereby decrease the polarization of naïve Th cells towards the Th2 phenotype (FIG. 7).

As far as the inventors know, this pathway provides the first example where ubiquitin-dependent protein degradation is regulated not via substrate protein phosphorylation but through the modulation of a substrate-specific E3 ligase. In other experiments, the inventors found that the Itch-dependent ubiquitination of c-Jun is not affected by elimination of the JNK phosphorylation sites and that T cells from c-JunAA mice, in which serines 63 and 73 of c-Jun [the JNK phosphorylation sites (Smeal et al. (1991) Nature 354, 494-496)], were replaced with alanines (Behrens et al. (1999) Nat Genet 21, 326-329), express lower levels of c-Jun relative to WT counterparts. These findings are consistent with both the previous suggestion that JNK-mediated phosphorylation can lead to reduced ubiquitination-dependent c-Jun degradation (Fuchs et al. (1996) Oncogene 13, 1531-1535; Musti et al. (1997) Science 275, 400-402), and with the substrate specificity of E3 ligases with WW domains, such as Itch, which is not directed toward phospho-epitopes (Joazeiro et al. (2000) Science 289, 2061-2062; D. Fang, unpublished data). Although the exact mechanism through which the MEKK1/JNK cascade regulates Itch turnover remains to be identified, the inventors note that it is cell type specific, as primarily lymphocytes, but negligibly in other cell types, of Mekk1^{KD} mice exhibit reduced Itch expression.

The inventors propose that the major biological function of the pathway through which MEKK1/JNK activation leads to accelerated Itch-dependent JunB turnover in T cells is to attenuate the polarization of naïve Th cells towards the Th2 phenotype in response to output from the TCR and co-stimulatory receptors. It was previously demonstrated that the extent and duration of TCR occupancy by antigen affects the differentiation of naïve Th cells into the Th1 and Th2 effector cell types or modulate the expression levels of critical Th1 and Th2 cytokines (Boyton et al. (2002) Trends Immunol 23, 526-529; Constant et al. (1995) J Exp Med 182, 1591-1596; Itoh et al. (1997) J Exp Med 186, 757-766; Kuchroo et al. (1995) Cell 80, 707-718; Lanzavecchia et al. (2000) Science 290, 92-97). Most relevant to inventors findings were the observations that high antigen doses polarize naïve Th cells towards the Th1 phenotype, while low doses of the same peptide antigen preferentially induced Th2 differentiation (Constant et al. (1995) J Exp Med 182, 1591-1596) and that T cells that express lower affinity TCRs may be more prone to differentiate towards the Th2 phenotype (Boyton et al. (2002) Trends Immunol 23, 526-529). There are also suggestions that co-engagement of CD28 may inhibit Th2 differentiation, while promoting Th1 differentiation (Kuchroo et al. (1995) Cell 80, 707-718). The mechanism through which the potency or duration of T cell activation affects effector functions is unknown. The inventors suggest that one such mechanism may entail MEKK1/JNK activation, which promotes Itch-dependent degradation of JunB, thereby inhibiting IL-4 production and formation of Th2 cells. Given that JNK activation requires co-engagement of both TCR and CD28 (Su et al. (1994) Cell 77, 727-736) or large Ca2+ transients (Dolmetsch et al. (1997) Nature 386, 855-858), it is certainly feasible that the MEKK1/JNK cascade is involved in interpreting the potency of the TCR signal. Therefore, strong JNK activation produced by intense or prolonged T cell stimulation is expected to inhibit differentiation to Th2 phenotype, a prediction that is entirely consistent with available results. It is noteworthy that differentiated Th2 cells exhibit reduced Ca2+ transients (Sloan-Lancaster et al. (1997) J Immunol 159, 1160-1168) and thus may be less capable of mounting a strong JNK activation response. Given the inventors' suggestion that strong or prolonged JNK activation may inhibit Th2 differentiation, these cells may have been selected to become Th2 cells because of their reduced Ca2+ signaling capacity.

It is not intended to convey that any of these cells, proteins, molecules, pathways, complexes, antibodies, and receptors have primarily one function. Physiological pathways are in flux, for example activation pathways, and not usually isolated from each other. There are several activation pathways leading towards cytokine production that overlap with several other pathways leading towards cell survival, proliferation, differentiation, activated cell death and apoptosis. For example, cytokine production pathways overlap in that one protein, such as MEKK1, under some circumstances contributes to increasing type 2 cytokine levels, such as when destabilized, while under other circumstances MEEK1 will contribute to decreasing type 2 cytokine levels such as when intact. The same is true for JNK1 and ITCH. Often these counteractive results are found in different cells types. Furthermore, compensatory mechanisms and/or redundancies within type 2 cytokine production pathways often counteract and/or mask the ability of any one protein to contribute to either T2 cytokine or T1 cytokine production. Therefore, the present invention is unique in clearly showing the contributions of MEEK1, JNK1 and ITCH towards increasing type 2 cytokine levels and the value thereof.

### Experimental

The following examples serve to illustrate certain preferred embodiments and aspects of the present invention and are not to be construed as limiting the scope thereof. In the experimental disclosure which follows, the following abbreviations apply: M (molar); mM (millimolar); μM (micromolar); nM (nanomolar); mol (moles); mmol (millimoles); μmol (micromoles); nmol (nanomoles); gm (grams); mg (milligrams); μg (micrograms); pg (picograms); L (liters); ml (milliliters); μl (microliters); cm (centimeters); mm (millimeters); μm (micrometers); nm (nanometers); ° C. (degrees Centigrade).

### EXAMPLE 1

Materials and Methods The following is a description of exemplary materials and methods that were used in subsequent Examples.

### Sources of Mice:

It is not intended to limit the source of mice. In one embodiment, mice were obtained by personal donations (for example, L. Chang for provided Jnk1^{-/-} mice and E. D. Gallagher provided anti-MEKK1 antibodies). In one embodiment, mice were obtained by engineering and breeding mice (for example, Mekk1^{KD/KD} mutant mice were generated by standard procedures from Mekk1^{KD} ES cells, in which the MEKK1 kinase domain was replaced with a β-galactosidase coding cassette (Xia et al., 2000). Chimeric mice were generated by injection of Mekk1^{KD} ES cells into C57BL/6 blastocysts. As used herein, the term blastocysts and blastocyst cells refers to a preimplantation embryo of 30-150 cells. A blastocysts contains a layer of specialized cells made up of trophoblasts which function to attach to the uterine wall and form the placenta. Inside the trophoblast layer is the undifferentiated inner cell mass. As used herein, the terms “ES” and “embryonic stem cells” refers to cells that contains the potential to differentiate into every cell type within a developing embryo. Embryonic Stem cells are derived from the inner cell mass of a blastocyst.

In one embodiment, Mekk^{KD/KD} mice were obtained by intercrossing Mekk1^{+/KD} mice. In another embodiment, Jnk1^{-/-} mice were bred as described by Sabapathy et al., 2001. In another embodiment, IL-4^{-/-} mice were obtained from the Jackson Laboratory. In one embodiment, similar results were obtained by analyzing the Mekk1^{ΔKD} mutation in a mixed 129×BL6 or pure BL6 background.

As used herein, the term chimeric refers to a composite of genetically distinct individuals, (e.g. following cell implantation from one animal into another, an allogeneic bone marrow graft, etc.).

As used herein, the terms “transgenic” and “mutant” when used in reference to an animal or to a cell refers to an animal or cell, respectively, which comprises one or more cells that contain a transgene, or whose genome has been altered by the introduction of a transgene. Transgenic cells, tissues and plants may be produced by several methods including the introduction of a “transgene” comprising nucleic acid (usually DNA) into a target cell or integration of the transgene into a chromosome of a target cell by way of human intervention, such as by the methods described herein. In one embodiment, knockout mice were of the C57BL/6 background. As used herein, the term “knockout” refers to a deletion or deactivation or ablation of a gene or deficient gene in a mouse or other laboratory animal or any cells in an animal. When said knockout includes the germ cells, subsequent breeding can create a line of animals that are incapable of or produce significantly less of said gene product. As used herein, the term “transgenic” or “mutant” when used in reference to a cell refers to a cell which contains a transgene, or whose genome has been altered by the introduction of a transgene.

### Flow Cytometry and Cell Isolation:

Single-cell suspensions of thymi and spleens were prepared from 6-8-week-old mice. Splenic CD4+ and CD8+ T cells were prepared using magnetic beads (Miltenyi Biotech). Monoclonal FITC-conjugated anti-CD8 and PE-conjugated anti-CD4 antibodies (PharMingen) were used for cell staining. Analyses were performed on a FACScan flow cytometer (Becton Dickinson) using CELL Quest software.

### T Cell Proliferation Assays

For proliferation assays, purified thymocytes or CD4⁺ or CD8⁺ splenic T cells (2×10⁵ cells/well) were cultured in round bottom 96-well plates (Costar) precoated with anti-CD3 antibody (BD Pharmingen) in the absence or presence of soluble anti-CD28 antibody (BD Pharmingen). After 3 days, cultures were pulsed for 6 hrs with 1 μCi [³H] thymidine (New England Nuclear) per well and cells were harvested. [³H] thymidine incorporation was measured by scintillation counting. When indicated, either recombinant IL-4 or neutralizing anti-IL-4 antibody (R&D Systems) were added prior to [³H] thymidine labeling.

### Thymocyte Proliferation and Survival Assays:

For proliferation assays, purified thymocytes (2×10⁵ cells/well) were cultured in round bottom 96-well plates (Costar) precoated with anti-CD3 antibody (BD Pharmingen) in the absence or presence of soluble anti-CD28 antibody (BD Pharmingen). After 3 days, cultures were pulsed for 6 hrs with 1 mCi [³H] thymidine (NEN) per well and cells were harvested. [³H] thymidine incorporation was measured by scintillation counting. When indicated, either recombinant IL-4 or neutralizing anti-IL-4 antibody (R&D Systems) was added to the cultures prior to addition of [³H] thymidine. For analysis of cell death, freshly isolated thymocytes were cultured in 24-well plates (Costar) precoated with anti-CD3 with or without soluble anti-CD28. At various time points after stimulation, viable thymocytes were identified by trypan blue exclusion. Apoptotic cells were identified by annexin-V and propidium iodide (Clontech) staining.

### Th Cell Differentiation and Activation

CD4⁺ T cells were purified from spleens of WT or Mekk1^{ΔKD} mice. Primary T cells were incubated with anti-CD3 (10 μg/ml) and mitomycin C (50 μg/ml)-treated WT antigen presenting cells (APC) in the presence of anti-IL-4 (2 μg/ml)+IL-12 (0.1 μg/ml) for Th1 differentiation or anti-IFNγ (1 μg/ml)+IL-4 (50 ng/ml) for Th2 differentiation. IL-2 (20 U/ml) was added to both the Th1 and Th2 cultures. After 7 days, Th subsets were collected, washed and restimulated with anti-CD3 (10 μg/ml)+anti-CD28 (1 μg/ml). Intracellular staining for IL-4 and IFN□ was performed using the cytofix/cytoperm kit (BD BioSciences).

### Plasmids, Transfections and Fusion Proteins:

Full-length or kinase domain-truncated MEKK1 cDNAs were described (Xia et al. (2000) Proc Natl Acad Sci USA 97, 5243-5248). The ubiquitin expression vector with a Myc or HA epitope tag was also described (Fang et al. (2001) J Biol Chem 276, 4872-4878). The C433A, F1443A and T1381A of MEKK1 mutants were generated by PCR mutagenesis and subcloned into the pCMV expression vector. WT or kinase dead JNKK2-JNK1 fusion proteins were described (Zheng et al. (1999) J Biol Chem 274, 28966-28971). c-Jun and JunB cDNAs were HA tagged as described and subcloned into the mammalian expression vector pEFneo (Invitrogen) (Fang et al. (2002) Nat Immunol 3, 281-287). WT or kinase dead JNKK2-JNK1 fusion proteins were described (Zheng et al. (1999) J Biol Chem 274, 28966-28971). c-Jun and JunB cDNAs were HA tagged and subcloned into the mammalian expression vector pEFneo (Invitrogen). WT and catalytically inactive Itch expression vectors were described (Qiu et al (2000) J Biol Chem 275, 35734-35737). 293T cells were cultured and transfected with various plasmids as described (Fang et al. (2001) J Biol Chem 276, 4872-4878). In one embodiment, A. Lin provided a JNKK2-JNK1 fusion protein. In one embodiment, D. Bohmann provided a Jun^{Ala} construct.

### Kinase Assays and Immunoblotting:

JNK1 was immunoprecipitated from 30 μg cell lysate with anti-JNK1 antibody (333, PharMingen) and its kinase activity measured by an immunecomplex kinase assay with GST-c-Jun(1-79) as a substrate (Xia et al. (2000) Proc Natl Acad Sci USA 97, 5243-5248). c-Jun phosphorylation was quantitated using a PhosphoImager (Bio-Rad) and gel loading was normalized by immunoblotting with a monoclonal antibody against JNK1/2 (666, PharMingen). ERK activation was examined by immunoblotting of gel separated whole cell extracts (100 μg) with an antibody specific for phospho-ERK (Cell Signaling). Polyclonal antibody against MEKK1 was prepared as described (Gallagher et al. (2002) J Biol Chem 277, 45785-45792). Antibodies against c-Jun, JunB, GATA3, c-Maf, NFATc1, NFATc2, p16, and p65 (RelA) were all from Santa Cruz Biotechnology. Polyclonal antibody against Itch was prepared as described (Qiu et al. (2000) J Biol Chem 275, 35734-35737). The levels of various proteins were quantitated by BioRad Quantity One software.

### RNA Analysis:

Total RNA from thymocytes was prepared using RNeasy kit (Qiagen). RNase protection analysis was performed using the RiboQuant multi-probe system (Pharmingen). Mouse cytokine multi-probe sets mCK1 and mJun/Fos were transcribed and radiolabeled using MAXIscript in vitro transcription kit (Ambion). Total RNA (2 mg) was hybridized to the probes and analyzed following the manufacturer's protocols. For real time PCR analysis, total thymocyte RNA (2 μg) was used to synthesize cDNA by SuperScript First-Strand Synthesis System (Invitrogen). cDNA products were resuspended in 200 ml of dH2O and 5 μl cDNA was used in a Real-Time PCR assay. PCR amplifications were performed in a total volume of 25 ml containing cDNA template, cytokine-specific primers, and Master Green SYBR Green reagent (Roche). Real-Time PCR reactions were performed in triplicates using an ABI Prism 7700 Sequence Detector (Applied Biosystems). The cytokine-specific primers used in this study were described (Giulietti et al. (2001) Methods 25, 386-401). Cyclophilin A mRNA was used for normalization.

### Pulse-Chase Experiments:

[³⁵S] labeling and pulse-chase experiments were performed as described (Fang et al. (2002) Nat Immunol 3, 281-287). Purified thymocytes were cultured in 24-well plates in Dulbecco's modified Eagle's medium (DMEM) lacking methionine and cysteine. Cells were stimulated with anti-CD3 (10 μg/ml)+anti-CD28 (1 μg/ml) for 24 hrs and then pulse-labeled for 1 hr by adding 100 μCi/ml [³⁵S] methionine and [³⁵S] cysteine (Amersham Biosciences). Cells were then chased for different times with cold amino acids and cell lysates were immunoprecipitated with anti-c-Jun, anti-JunB, anti-RelA (p65) (all from Santa Cruz) and anti-Itch (Qiu et al. (2000) J Biol Chem 275, 35734-35737). The immunecomplexes were separated by SDS-PAGE and labeled proteins were detected by autoradiography.

### Chromatin Immunoprecipitation Assays (ChIP):

ChIP assays were carried out as described. (Saccani et al. (2002) Genes Dev 16, 2219-2224). Polyclonal antibodies against c-Jun (H-79) and JunB (C-11) were from Santa Cruz. The primers for amplifying the IL-4 promotor region were 5′-GTTGCTGAAACCAAGGGAAA-3′ (SEQ ID NO:01) and 5′-TGAAAGGCCGATTATGGTGT-3′ (SEQ ID NO:02). The primers for amplifying the IL-4 coding region were 5′-TCAACCCCCAGCTAGTTGTC-3′ (SEQ ID NO:03) and 5′-AAATATGCGAAGCACCTTGG-3′ (SEQ ID NO:04).

In vitro Ubiquitination Assays

His-tagged ubiquitin, E1, and GST-Ubc7 (E2) were prepared as described (Joazeiro et al. (2000) Science 289, 2061-2062) and used in in vitro ubiquitination assays (Qiu et al. (2000) J Biol Chem 275, 35734-35737). Ubiquitination reaction mixtures containing E1 (100 nM), Ubc7 (0.5 μM), ubiquitin (5 μM), ATP (2 mM), GST-c-Jun (2 μg), and immunoprecipitated Itch on Sepharose beads were incubated at 25° C. for 90 minutes. Anti-c-Jun antibody was used for immunoblotting. To examine Itch autoubiquitination, reactions were incubated in the absence of GST-c-Jun and analyzed by immunoblotting with an anti-ubiquitin or anti-Itch antibodies after re-precipitation of Itch. Omission of E1, E2, or ATP prevented Itch or c-Jun ubiquitination.

### EXAMPLE 2

### Hyperproliferation of Mekk1^{KD} Mutant T Cells

Growth and differentiation tests were done to compare wildtype T cells to mutant T cells deficient in MEKK1 kinase.
A. Thymocytes from WT and Mekk1^{KD} mice were incubated with the indicated concentrations (μg/ml) of anti-CD3 or anti-CD3+anti-CD28 for 72 hrs. Cell proliferation was measured by [³H] thymidine incorporation. Results are averages of 6 experiments. (FIG. 1A)
B. Thymocytes from WT and Mekk1^{KD} mice were cultured with 5 μg/ml of anti-CD3 with or without 0.5 μg/ml of anti-CD28. At the indicated times, cell viability was determined by trypan blue staining. Values represent the mean proportion of viable thymocytes relative to untreated cultures (100%) in 3 separate experiments. (FIG. 1B)
C. CD4+ and CD8+ splenic T cells from WT and Mekk1^{KD} mice were treated with 10 μg/ml anti-CD3 and 1 μg/ml anti-CD28 for 48 hrs. Cell proliferation was measured as above. Results are averages of 3 experiments. (FIG. 1C)

While it is not intended the present invention be limited by the degree to which MEKK1 kinase-deficient (Mekk1^{KD}) thymocytes are responsive to TCR engagement, these data show that MEKK1 kinase-deficient (Mekk1^{KD}) thymocytes were hyperresponsive to TCR engagement with anti-CD3 antibody and this hyperresponsiveness was strongly augmented by ligation of the co-stimulatory CD28 receptor. In addition, activation-induced death was slightly elevated in mutant cells while differentiation appeared unaffected.

### EXAMPLE 3

### Reduced JNK Activity Results in Increased Thymocyte Proliferation

The effect of the Mekk1^{KD} mutation on activation of JNK and other MAPKs in response to TCR and CD28 engagement was investigated.
A. WT and Mekk1^{KD} thymocytes were incubated with anti-CD3 (10 μg/ml) and anti-CD28 (1 μg/ml). At the indicated times, JNK activity was measured by an immunecomplex kinase assay using GST-c-Jun(1-79) as the substrate. Phosphorylated c-Jun was detected by autoradiography and quantitated using a PhosphorImager. The level of immunoprecipitated JNKs was determined by immunoblotting. This experiment was repeated several times with similar results. (FIG. 2A)
B. WT and Mekk1^{KD} thymocytes were stimulated as above. At the indicated times, ERK activation was examined by immunoblotting with an antibody against phosphorylated ERK. The same membrane was reprobed with a general anti-ERK antibody. (FIG. 2B)
C. Thymocytes were incubated with indicated concentrations (μg/ml) of anti-CD3 and anti-CD28 in the presence of increasing concentrations (mM) of the JNK inhibitor SP600125 (SP) for 72 hrs. Cell proliferation was measured by [³H] thymidine incorporation. (FIG. 2C)
D. Thymocytes from Jnk1^{±} and Mekk1^{+/KD}Jnk1^{±} mice were treated with indicated concentrations (μg/ml) of anti-CD3 with or without anti-CD28 for 72 hrs. Cell proliferation was measured as above. (FIG. 2D)

The results suggest that Mekk1^{+/KD}Jnk1^{±} thymocytes exhibited a pronounced hyperresponsiveness to TCR and CD28 engagement in comparison to either Jnk1^{±} thymocytes.

### EXAMPLE 4

### Enhanced Th2 Cytokine Production by Mekk1^{KD} Thymocytes

The role of cytokine gene expression in the hyperresponsive phenotype of Mekk1^{KD} T cells was investigated. WT and Mekk1^{KD} thymocytes were stimulated with anti-CD3 (5 μg/ml) and anti-CD28 (0.5 μg/ml) for 48 hrs. Expression of cytokine mRNAs was measured by RNase protection. The levels of ribosomal protein L32 and GAPDH mRNAs were used as loading controls. (FIG. 3A)
B. WT and Mekk1^{KD} thymocytes were stimulated as above. The levels of various cytokines mRNAs were quantitated by Real-time PCR and normalized to the level of cyclophilin A mRNA. (FIG. 3B)
C. Mekk1^{KD} thymocytes were incubated with indicated concentrations (μg/ml) of anti-CD3 or anti-CD3+anti-CD28 for 72 hrs in the presence of increasing amounts of anti-IL-4. Cell proliferation was measured by [³H] thymidine incorporation. (FIG. 3C)
D. WT thymocytes were incubated with the indicated concentrations (μg/ml) of anti-CD3 or anti-CD3+anti-CD28 for 72 hrs in the presence of increasing amounts of IL-4. Cell proliferation was measured as above. (FIG. 3D)

These results suggest that MEKK1 plays a role in determining the expression of T2 cytokines.

### EXAMPLE 5

Upregulation of JunB and c-Jun Protein Levels in Mekk1^{KD} Thymocytes

The expression of transcriptional regulators and their effects on the IO-4 promoter were investigated in MEKK1 mutant thymocytes.
A. WT and Mekk1^{KD} thymocytes were stimulated with anti-CD3 (5 μg/ml)+anti-CD28 (0.5 μg/ml) for 24 hrs. Cell extracts were prepared and the levels of various transcription factors were measured by immunoblotting and quantitated using a PhosphoImager. The relative levels of the different proteins are indicated. (FIG. 4A)
B. WT and Mekk1^{KD} thymocytes were stimulated as described above. mRNA levels of Jun family members were measured by RNase protection. (FIG. 4B)
C. WT and Mekk1^{KD} thymocytes were stimulated as above. Cells were collected and recruitment of c-Jun and JunB to the indicated regions of the IL-4 gene was examined by ChIP experiments. (FIG. 4C)

These data show that JunB and c-Jun expression are increased in Mekk1^{KD} thymocytes.

### EXAMPLE 6

MEKK1 Promotes Ubiquitin-Dependent Degradation of c-Jun and JunB
A. WT and Mekk1^{KD} thymocytes were pulse-labeled with medium containing [³⁵S] cysteine and methionine for 1 hr in the presence of anti-CD3 (5 μg/ml)+anti-CD28 (0.5 μg/ml). Cells were chased with medium containing non-labeled amino acids for the indicated times. Cell lysates were immunoprecipitated with various antibodies as indicated, separated by SDS-PAGE and subjected to autoradiography. The amounts of radioactivity were quantitated using a PhosphoImager. This experiment was repeated twice with similar results. (FIG. 5A)
B. The data in panel A were quantitated and normalized. The relative amounts of each [³⁵S]-labeled protein at t=0 was considered to be 100%. (FIG. 5B)
C. 293T cells were transiently transfected with plasmids encoding HA-tagged c-Jun or JunB, WT MEKK1 or a kinase domain-truncated MEKK1 (mt), and Myc-Ub. c-Jun and JunB were immunoprecipitated and Ub conjugation was examined with anti-Myc. The same membrane was reprobed for JunB and c-Jun. Data are shown for JunB. This experiment was repeated twice with similar results. (FIG. 5C)

### EXAMPLE 7

### MEEK1/JNK Signaling Pathway Regulates Itch and Jun Turnover

Elevated expression of c-Jun and JunB and increased Th2 cytokine production are observed in T cells from Itchy mutant mice, that express an inactive form of the Itch E3 ubiquitin protein ligase (Fang et al, 2003). While it is not intended that the present invention be limited to any specific mechanism, these similarities suggest that MEKK1-directed JunB (or c-Jun) turnover is Itch-dependent and that the MEKK1/JNK cascade controls Itch activity or expression.
A. WT and Mekk1^{KD} thymocytes were cultured in the presence of anti-CD3 (5 μg/ml)+anti-CD28 (0.5 μg/ml) for 24 hrs. The levels of Itch and actin were examined was measured by immunoblotting. (FIG. 6A)
B. Thymocytes were lysed and immunoprecipitated with indicated antibodies. The immunecomplexes were separated by SDS-PAGE and immunoblotted with anti-MEKK1 and anti-Itch. The positions of MEKK1 and IgG heavy chain are indicated. This experiments was repeated 3 times with similar results. (FIG. 6B)
C. WT and Mekk1^{KD} thymocytes were pulse-labeled with [³⁵S] cysteine and methionine for 1 hr in the presence of anti-CD3 (5 μg/ml)+anti-CD28 (0.5 μg/ml). Cells were chased with nonradioactive amino acids for the indicated times, at which they were lysed and Itch expression levels were determined by radioimmunoprecipitation. The levels of [³⁵S]-labeled Itch were quantitated using a PhosphoImager. (FIG. 6C)
D. WT and Mekk1^{KD} thymocytes were stimulated as above for 24 hrs in the absence or presence (0.5 μM) of the JNK inhibitor SP600125 (SP). Cell lysates were prepared and the levels of the different proteins were examined by immunoblotting. (FIG. 6D)
E. Thymocytes were stimulated for 5 min in the absence or presence (0.5 μM) of the JNK inhibitor (SP). JNK activity was measured by an immunecomplex kinase assay. Phosphorylated c-Jun was detected by autoradiography and quantitated using a PhosphoImager. (FIG. 6E)
F. 293T cells were transfected with plasmids encoding HA-Ub and Myc-tagged c-Jun or JunB. Cells were incubated with (1.0 μM) or without the JNK inhibitor (SP) for 24 hrs. The Jun proteins were immunoprecipitated and Ub conjugation was examined as described above. (FIG. 6F)

An important role in protein ubiquitination is played by the E3 ubiquitin protein ligases, which are responsible for substrate recognition (Hershko et al. (1998) Annu Rev Biochem 67, 425-479).

Moreover these data show the absence of MEKK1 kinase activity results in destabilization of Itch, while it stabilizes c-Jun and JunB. Further, incubation of activated WT thymocytes with SP600125 resulted in a 4-fold increase in c-Jun and JunB expression and a 2-fold decrease in Itch expression after 24 hrs while an effect of SP600125 on Jun and Itch expression correlated with its effect of JNK activation and that treatment with the JNK inhibitor reduced the extent of c-Jun and JunB polyubiquitination in transfected cells.

### EXAMPLE 8

### MEKK1/JNK Signaling Pathway Negatively and Positively Regulates JunB Stability and Activity in Response to T Cell Stimulation

Engagement of TCR and CD28 co-stimulatory receptor results in activation of the MEKK1 to MKK4/MEKK7 to JNK1/2 signaling cascade. Transcriptional activity of JunB is enhanced by this pathway through direct phosphorylation by JNKs and leads to transcription of IL-4, a key cytokine for Th2 cells. On the other hand, the MEKK1/JNK pathway regulates the stability of the E3 ubiquitin protein ligase Itch, which promotes the polyubiquitination and degradation of JunB. This pathway, which dominates under intense T cell activation conditions, inhibits Th2 cytokine production and Th2 differentiation. (FIG. 7)

### EXAMPLE 9

### Reduced JNK Activation in Mekk1^{ΔKD} Mutant T Cells Results in Hyper-Responsiveness

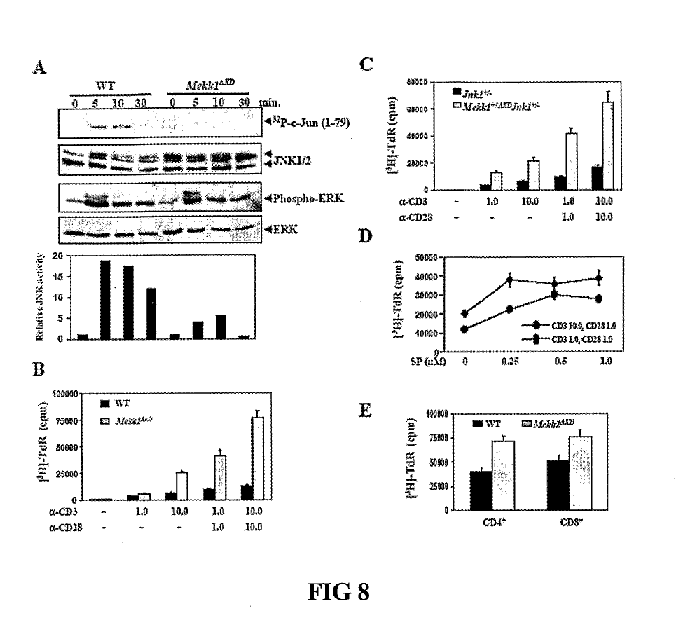

Loss of MEKK1 catalytic activity resulted in a decrease in JNK activation following stimulation of thymocytes with anti-CD3+anti-CD28 (FIG. 8A). By contrast, ERK activation was not affected. p38 MAPK was not activated by these stimuli in either WT or Mekk1^{ΔKD} thymocytes. Similar results were obtained when splenic T cells were examined.

Loss of MEKK1 catalytic activity had little effect on thymocyte numbers and differentiation into the CD4 and CD8 double and single positive classes (unpublished results). However, Mekk1^{ΔKD} thymocytes were hyper-responsive to TCR engagement with anti-CD3 antibody and this hyper-responsiveness was strongly augmented by ligation of the co-stimulatory CD28 receptor (FIG. 8B). Proliferation in response to PMA and ionomycin, pharmacological stimuli that bypass TCR and CD28, was not affected. The increased proliferative response was not due to decreased activation-induced death, as cell viability was not changed in Mekk1^{ΔKD} thymocytes. In addition, the expression levels of several cell death regulators were unchanged.

Mekk1^{+/ΔKD} Jnk1^{±} thymocytes exhibited a pronounced hyper-responsiveness to TCR and CD28 engagement in comparison to either Jnk1^{±} (FIG. 8C) or Mekk1^{+/ΔKD} thymocytes. To confirm that loss of JNK activity causes TCR hyper-responsiveness, the small molecule JNK inhibitor SP600125 was used. See, (Bennett et al. (2001) Proc Natl Acad Sci USA 98, 13681-13686). Stimulation of WT thymocytes with different concentrations of anti-CD3+anti-CD28 in the presence of increasing amounts of SP600125, which inhibited JNK activity (see below), resulted in at least 2-fold increase in [³H] thymidine incorporation (FIG. 8D). On the other hand, the p38 inhibitor SB202190 (Lee et al. (1994) Nature 372, 739-746) did not produce such an effect. Similar, but less pronounced, hyper-responsiveness was observed in peripheral Mekk1^{ΔKD} T cells (FIG. 8E), whereas the rate of cell death was comparable between WT and Mekk1^{ΔKD} splenic T cells.

### Reduced JNK Activity and Increased T Cell Proliferation in Mekk1^{ΔKD} T Cells

A. WT and Mekk1^{ΔKD} thymocytes were incubated with anti-CD3 (10 μg/ml)+anti-CD28 (1 μg/ml). At the indicated times, JNK activity was measured by an immunecomplex kinase assay with GST-c-Jun(1-79) as the substrate. Phosphorylated c-Jun was detected by autoradiography and quantitated using a PhosphorImager. The level of immunoprecipitated JNKs was determined by immunoblotting. ERK activation was examined by immunoblotting with anti-phospho-ERK. The same membrane was reprobed with a general anti-ERK antibody. (FIG. 8A)
B. WT and Mekk1^{ΔKD} thymocytes were incubated with the indicated concentrations (μg/ml) of anti-CD3 or anti-CD3+anti-CD28 for 72 hrs. Cell proliferation was measured by [³H] thymidine incorporation. Results are averages of 6 experiments.
C. Jnk1^{±} and Mekk1^{+/ΔKD} Jnk1^{±} thymocytes were treated as above and cell. (FIG. 8B) proliferation was examined. Results are averages of 3 experiments. (FIG. 8C)
D. WT thymocytes were incubated with indicated concentrations (μg/ml) of anti-CD3+anti-CD28 in the presence of increasing concentrations (μM) of the JNK inhibitor SP600125 (SP) for 72 hrs. Cell proliferation was measured as above. Results are averages of 3 experiments. (FIG. 8D)
E. CD4⁺ and CD8⁺ splenic T cells from WT and Mekk1^{ΔKD} mice were treated with anti-CD3 (10 μg/ml)+anti-CD28 (1 μg/ml) for 48 hrs. Cell proliferation was measured as above. Results are averages of 3 experiments. (FIG. 8E)

### EXAMPLE 10

### Increased Th2 Cytokine Production by Mekk1^{ΔKD} T Cells

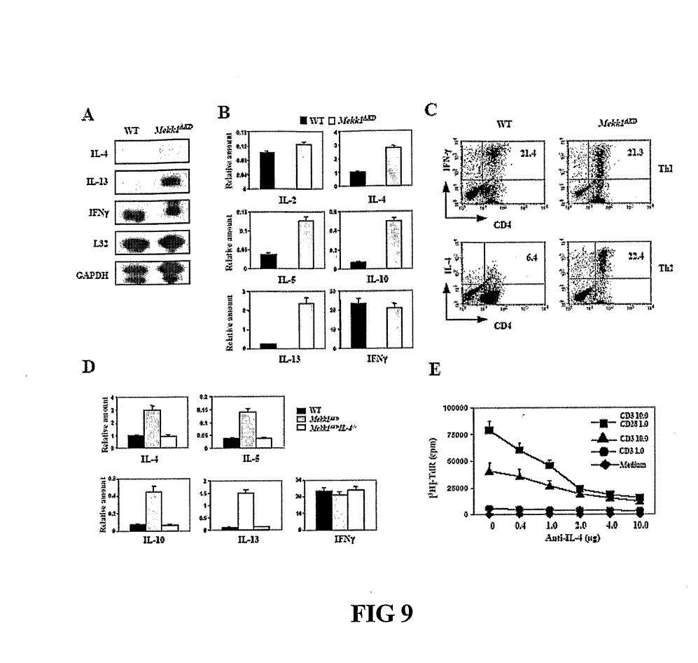

Mekk1^{ΔKD} thymocytes express elevated levels of IL-4 and IL-13 mRNAs but close to normal levels of interferon γ (IFNγ) mRNA after stimulation with anti-CD3+anti-CD28 (FIG. 9A). Unstimulated thymocytes expressed background levels of these mRNAs. In addition to ribonuclease (RNase) protection analysis a real-time PCR assay was used to quantitate the levels of these and other mRNAs in CD4⁺ T cells from WT and Mekk1^{ΔKD} mice after 24 hrs stimulation with anti-CD3+anti-CD28. Mekk1^{ΔKD} T cells expressed up to 5-fold more IL-4, IL-5, IL-10 and IL-13 mRNAs (FIG. 9B). Up-regulation of these mRNAs was also observed in Mekk1^{ΔKD} thymocytes using Real-time PCR.

The cytokine mRNAs that are overexpressed in Mekk1^{ΔKD} CD4⁺ T cells are characteristic of the Th2 effector cell type (Murphy et al. (2000) Annu Rev Immunol 18, 451-494; Paul et al. (1994) Cell 76, 241-251). Therefore, MEKK1 catalytic activity may be important in differentiation of primary T cells into the Th1 and Th2 effector subsets. To test this hypothesis, the inventors cultured naïve WT or Mekk1^{ΔKD} CD4⁺ T cells under Th1- or Th2-polarization conditions for 7 days. The cells were restimulated with anti-CD3+anti-CD28, and the outcome was examined by intracellular staining for IL-4 and IFNγ. Deletion of the MEKK1 catalytic domain strongly enhanced the generation of IL-4⁺ Th2 cells but had negligible effect on generation of IFNγ⁺ Th1 cells (FIG. 9C).

To determine whether the elevated Th2 response of Mekk1^{ΔKD} CD4⁺ T cells is IL-4-dependent, the inventors crossed IL-4-^{-/-} mice (Metwali et al. (1996) J Immunol 157, 4546-4553) with Mekk1^{ΔKD} mice. The inventors found that the elevated production of Th2-type cytokines seen in Mekk1^{ΔKD}IL-4^{+/+} T cells was reduced to normal in Mekk1^{ΔKD}IL-4^{-/-} cells (FIG. 9D). To determine whether increased IL-4 production was also responsible for the hyperproliferation of Mekk1^{ΔKD} T cells, the inventors examined the effect of a neutralizing anti-IL-4 antibody as well as the IL-4 gene deletion. Incubation with anti-IL-4, but not control anti-IgG, markedly reduced the proliferation of Mekk1^{ΔKD} thymocytes elicited by anti-CD3 in the absence or presence of anti-CD28 (FIG. 9E). Similar results were obtained upon deletion of the IL-4 gene. Further, addition of exogenous IL-4 to WT thymocytes strongly potentiated their proliferative response.

### Enhanced Th2 Cytokine Production and Skewed Differentiation by Mekk1^{ΔKD} T Cells

A. WT and Mekk1^{ΔKD} thymocytes were stimulated with anti-CD3 (5 μg/ml)+anti-CD28 (0.5 μg/ml) for 48 hrs. Expression of cytokine mRNAs was measured by RNase protection. The levels of ribosomal protein L32 and GAPDH mRNAs were used as loading controls. (FIG. 9A)
B. CD4⁺ T cells from WT and Mekk1^{ΔKD} mice were stimulated with anti-CD3 (5 μg/ml)+anti-CD28 (0.5 μg/ml) for 24 hrs. The levels of cytokine mRNAs were quantitated by Real-time PCR and normalized to the level of cyclophilin A mRNA. The relative amounts of IL-4 mRNA in WT cells were given an arbitrary level of 1.0. (FIG. 9B)
C. CD4⁺ T cells from WT and Mekk1^{ΔKD} mice were cultured under Th1- or Th2-polarizing conditions for 7 days. Th subsets were restimulated with anti-CD3 (10 μg/ml)+anti-CD28 (1 μg/ml) for 6 hrs, and analyzed for cytokine expression by flow cytometry. (FIG. 9C)
D. CD4⁺T cells from WT, Mekk1^{ΔKD}, and Mekk1^{ΔKD}IL-4^{-/-} mice were treated with anti-CD3 (5 μg/ml)+anti-CD28 (0.5 μg/ml) for 24 hrs. The levels of cytokine mRNAs were quantitated by Real-time PCR and normalized to the level of cyclophilin A mRNA. The relative amounts of IL-4 mRNA in WT cells were given an arbitrary level of 1.0. (FIG. 9D)
E. Mekk1^{ΔKD} thymocytes were incubated with indicated concentrations (μg/ml) of anti-CD3 or anti-CD3+anti-CD28 for 72 hrs in the presence of increasing amounts of anti-IL-4 and cell proliferation was examined. (FIG. 9E)

### EXAMPLE 11

Elevated c-Jun and JunB Protein Accumulation in Mekk1^{ΔKD} Cells

Multiple transcription factors, including NF-AT, GATA-3, c-Maf, and JunB, are involved in IL-4 gene induction (Murphy et al. (2000) Annu Rev Immunol 18,451-494; Paul et al. (1994) Cell 76, 241-251). JunB (Li et al. (1999) Embo J 18, 420-432), as well as its relative c-Jun (Hibi et al. (1993) Genes Dev 7, 2135-2148; Kallunki et al. (1996) Cell 87, 929-939), are targets for JNK-mediated phosphorylation.

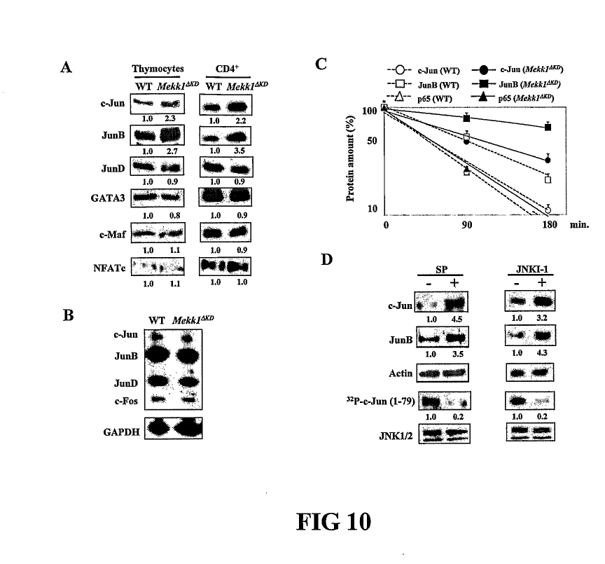

The effect of the mutation on the levels and subcellular distribution of these transcription factors was evaluated. Both c-Jun and JunB were elevated in activated thymocytes and CD4⁺ T cells from Mekk1^{ΔKD} mice, while expression of JunD, another family member, was unchanged (FIG. 10A). Mekk1^{ΔKD} cells, also expressed normal levels of GATA-3, c-Maf, NF-ATc1 and NF-ATc2. In addition, nuclear translocation of NF-ATs appeared to be normal in Mekk1^{ΔKD} T cells.

Despite a change in protein levels, the levels of c-Jun and JunB mRNAs were not different between WT and mutant thymocytes (FIG. 10B). In WT thymocytes, newly synthesized c-Jun and JunB proteins were degraded with half-lives (t1/2) of 59 and 78 min, respectively (FIG. 10C). Both proteins were significantly more stable in Mekk1^{ΔKD} thymocytes, where their t1/2 were extended to 109 and 280 min, respectively. By contrast, the turnover of RelA (p65) was unaltered between WT and Mekk1^{ΔKD} cells. To test whether the effect of MEKK1 on Jun protein turnover was mediated through JNK, two different JNK inhibitors were used. Incubation of activated WT thymocytes with either SP600125 or JNKI-1, a peptide inhibitor based on the c-Jun docking site for JNK (Borsello et al. (2003) Nat Med 9, 1180-1186; Kallunki et al (1996) Cell 87, 929-939), resulted in about a 4-fold increase in c-Jun and JunB expression (FIG. 10D). Negligible such effects were observed upon incubation with the p38 inhibitor SB202190. The effect of the JNK inhibitors on Jun protein levels correlated with their effects on JNK activation. In addition to the effects on Jun expression, treatment of CD4⁺ T cells with either JNK inhibitor resulted in increased IL-4 mRNA induction.

Inactivation of MEKK1 or JNK Slows Down JunB and c-Jun Turnover
A. Thymocytes and CD4⁺ T cells from WT and Mekk1^{ΔKD} were stimulated with anti-CD3+anti-CD28 for 24 hrs. Cell extracts were prepared and the levels of various transcription factors were measured by immunoblotting. The relative levels of the different proteins determined by densitometry are indicated. (FIG. 10A)
B. WT and Mekk1^{ΔKD} thymocytes were stimulated as above. The mRNA levels of Jun family members and c-Fos were measured by RNase protection. The levels of GAPDH mRNA were used as loading control. (FIG. 10B)
C. WT and Mekk1^{ΔKD} thymocytes were pulse-labeled with [³⁵S] cysteine and methionine for 1 hr in the presence of anti-CD3+anti-CD28 after one day incubation with these antibodies. Cells were chased with excess non-labeled amino acids for the indicated times. Cell lysates were prepared and immunoprecipitated with various antibodies as indicated, separated by SDS-PAGE and analyzed by autoradiography. The amounts of radioactivity in each protein band were quantitated using a PhosphorImager. The relative amounts of each [³⁵S]-labeled protein at t=0 was considered to be 100%. The results are averages of 3 separate experiments. (FIG. 10C)
D. WT and Mekk1^{ΔKD} CD4⁺ T cells were stimulated as above in the absence or presence of the JNK inhibitor SP600125 (0.5 μM) or JNKI-1 (25 μM) for 24 hrs. Cell extracts were prepared and the levels of indicated proteins were measured by immunoblotting. JNK activity was measured by an immunecomplex kinase assay. The relative levels of the different proteins relative to actin and JNK1/2 levels are indicated. (FIG. 10D)

### EXAMPLE 12

### The MEKK1-JNK Pathway Enhances the E3 Ubiquitin-Ligase Activity of Itch

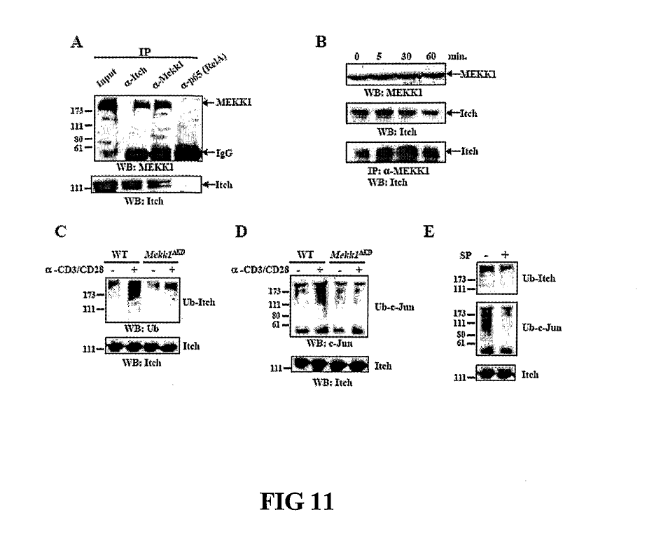

Itch and MEKK1 demonstrated an efficient and nearly quantitative interaction in both thymocytes and splenic T cells (FIG. 11A). Although the expression levels of Itch and MEKK1 were not modulated, the interaction between Itch and MEKK1 was enhanced after TCR activation (FIG. 11B). Loss of MEKK1 or JNK activity did not prevent the formation of the MEKK1-Itch complex.

Next, the inventors investigated whether the MEKK1-JNK signaling cascade regulates the E3 ligase activity of Itch or its ability to recognize c-Jun and JunB. Itch was immunoprecipitated from: anti-CD3+anti-CD28 stimulated and unstimulated WT and Mekk1^{ΔKD} T cells. Ubiquitination of Itch was analyzed by an in vitro ubiquitination assay. As shown by the slower migrating bands detected by immunoblotting with anti-ubiquitin antibody, the ubiquitination of Itch was stimulated by TCR activation (FIG. 11C). However, this effect was markedly reduced in Mekk1^{ΔKD} T cells. Ubiquitination of Itch was E1 and E2 dependent as omission of E1, E2, or ATP prevented Itch ubiquitination.

To further examine whether the MEKK1-JNK cascade regulates Itch E3 activity towards Jun proteins, purified GST-c-Jun fusion protein was incubated with immunoprecipitated Itch in the presence of E1, E2, and ubiquitin. As revealed by immunoblotting with anti-c-Jun antibody, Itch isolated from primary T cells mediated polyubiquitination of c-Jun in a manner dependent on E1 and E2. The E3 activity of Itch towards c-Jun was enhanced upon TCR and CD28 co-ligation of WT T cells, while such an increase in Itch activity was not found in Mekk1^{ΔKD} T cells (FIG. 11D). The effect of the JNK inhibitor SP600125 on the regulation of Itch activity was also examined. Both Itch autoubiquitination and c-Jun polyubiquitination were significantly reduced in anti-CD3+anti-CD28 stimulated T cells pretreated with the JNK inhibitor (FIG. 11E). Similar results were also obtained with the peptide inhibitor of JNK. These data show that E3 ubiquitin ligase activity of Itch is strongly enhanced in response to TCR and CD28 activation of T cells and that the MEKK1-JNK pathway is involved in this response.

### A MEKK1-JNK Cascade Enhances the E3 Ubiquitin Ligase Activity of Itch

A. Non-stimulated WT T cells were lysed and immunoprecipitated with the indicated antibodies. The immunecomplexes were gel separated and immunoblotted with anti-MEKK1 or anti-Itch antibodies. The positions of MEKK1, Itch and IgG heavy chain are indicated. (FIG. 11A)
B. WT T cells were stimulated with anti-CD3 (10 μg/ml)+anti-CD28 (1 μg/ml) for the indicated times. Cell extracts were prepared and the levels of MEKK1 and Itch were measured by immunoblotting. Cell lysates were also immunoprecipitated with anti-MEKK1 and immunoblotted with anti-Itch as above. (FIG. 11B)
C. WT and Mekk1^{ΔKD} T cells were left unstimulated or stimulated as above for 15 min. Cell extracts were prepared and Itch was isolated by immunoprecipitation. Itch was incubated with ubiquitin, E1, E2, and ATP at 25° C. for 90 min. After reprecipitation of Itch, reaction mixtures were separated by SDS-PAGE and immunoblotted with anti-ubiquitin antibody. The same membrane was reprobed with anti-Itch antibody. The positions of non-ubiquitinated and ubiquitinated Itch are indicated. (FIG. 11C)
D. WT and Mekk1^{ΔKD} T cells were treated as above. Itch immunecomplexes were isolated as above and incubated with ubiquitin, E1, E2, ATP and purified GST-c-Jun at 25° C. for 90 min. The reaction mixtures were separated by SDS-PAGE and immunoblotted with anti-c-Jun. The same membrane was reprobed with anti-Itch. (FIG. 11D)
E. WT T cells were stimulated as above in the absence or presence of the JNK inhibitor SP600125 (1 μM) for 15 min. In vitro ubiquitination assays using immunoprecipitated Itch as the E3 ubiquitin ligase with or without GST-c-Jun as the substrate were performed and analyzed as above. (FIG. 11E)

### EXAMPLE 13

MEKK1-JNK Cascade Promotes Itch-Dependent c-Jun and JunB Ubiquitination in Living Cells

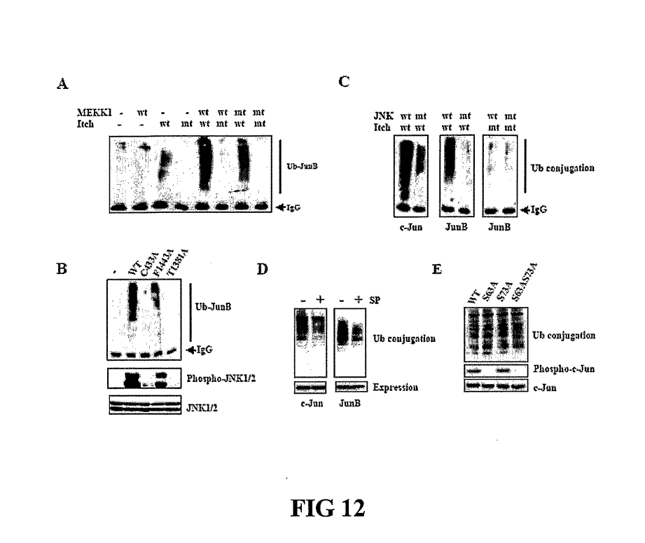

293T cells were transfected with plasmids encoding hemagglutinin (HA)-tagged ubiquitin, Myc-tagged JunB or c-Jun, WT or kinase domain-truncated MEKK1, similar to the MEKK1 polypeptide expressed in Mekk1^{ΔKD} cells, and either WT or a catalytically inactive Itch mutant (Qiu et al. (2000) J Biol Chem 275, 35734-35737). Cell lysates were immunoprecipitated with anti-Myc antibody, and ubiquitination of c-Jun and JunB was monitored by immunoblotting of gel-separated proteins with anti-HA. Ectopic expression of WT MEKK1 promoted the polyubiquitination of both c-Jun and JunB in a manner highly dependent on expression of WT Itch (FIG. 12A). Overexpression of kinase-deleted MEKK1 did not enhance the extent of Itch-dependent Jun polyubiquitination. The inactive Itch mutant acted in a dominant-negative manner and reduced both basal and MEKK1-stimulated Jun polyubiquitination. These data indicate that MEKK1 can enhance Itch-mediated Jun ubiquitination in cells.

293T cells were cotransfected as above with HA-tagged ubiquitin, Myc-JunB, Itch as well as WT and mutant versions of MEKK1. The C433A mutation lies in the PHD domain of MEKK1 and reduces its kinase activity (FIG. 12B). The F1442A mutation lies within the kinase domain of MEKK1 and has a small negative effect on its kinase activity whereas the adjacent T1381A mutation results in complete loss of kinase activity (FIG. 12B). The expression levels of three mutants are similar to those of WT MEKK1 (E. G., unpublished results). As before, WT MEKK1 strongly enhanced the polyubiquitination of JunB, while less potent enhancement was produced by the F1443 mutant (FIG. 5B). On the other hand, hardly any increase in JunB polyubiquitination was seen upon expression of the C433A and T1381A mutants. These data show that the ability of MEKK1 to activate JNK correlates with its ability to enhance Itch-dependent Jun ubiquitination.

To confirm the role of JNK in Itch-dependent Jun ubiquitination, 293T cells were cotransfected with HA-ubiquitin, Myc-tagged c-Jun or JunB, WT or mutant Itch, and either active (WT) or inactive (mt) JNKK2-JNK1 fusion proteins (Zheng et al. (1999) J Biol Chem 274, 28966-28971). Overexpression of the active JNKK2-JNK1 fusion protein enhanced Itch-dependent polyubiquitination of both c-Jun and JunB, while the inactive JNKK2-JNK1 fusion protein did not display such an activity (FIG. 12C). Treatment with the JNK inhibitor reduced the extent of Itch-induced c-Jun and JunB polyubiquitination in transfected cells (FIG. 12D).

293T cells were transfected with HA-ubiquitin, Itch, MEKK1, and WT and mutant versions of c-Jun that either lacked one or both of the known JNK phosphorylation sites (Smeal et al. (1991) Nature 354, 494-496). Consistent with the independence of HECT domain ligases of substrate phosphorylation (Ciechanover et al. (2000) Bioessays 22, 442-451; Joazeiro et al. (2000) Science 289, 2061-2062), the N-terminal phosphorylation of c-Jun by JNK had negligible effect on the extent of Itch-induced polyubiquitination (FIG. 125E). Thus, JNK-mediated phosphorylation enhances Jun ubiquitination through enhancement of Itch catalytic activity.
A. 293T cells were transiently transfected with plasmids encoding HA-tagged ubiquitin, Myc-tagged JunB, WT or a kinase domain-deleted MEKK1 (mt), and WT or catalytically inactive (mt) Itch. After 24 hrs, JunB was immunoprecipitated and gel separated, and ubiquitin conjugation was examined by immunoblotting with anti-HA antibody. (FIG. 12A)
B. 293T cells were cotransfected with HA-ubiquitin, Myc-JunB, Itch and the indicated MEKK1 constructs. JunB ubiquitination was examined as above. JNK activation was examined by immunoblotting with an antibody against endogenous phopho-JNK1/2. The same membrane was reprobed with a general anti-JNK1/2 antibody. (FIG. 12B)
C. 293T cells were cotransfected with HA-ubiquitin, Myc-tagged c-Jun or JunB, WT or catalytically inactive (mt) Itch, and WT or inactive JNKK2-JNK1 (mt) fusion proteins. Jun ubiquitination was examined as above. (FIG. 12C)
D. 293T cells were transfected as above. Cells were incubated with (1.0 μM) or without the JNK inhibitor SP600125 for 24 hrs. Jun proteins were immunoprecipitated and their ubiquitination was examined as above. (FIG. 12D)
E. 293T cells were transfected as above including WT and phosphorylation-deficient c-Jun constructs. c-Jun ubiquitination was analyzed as above. c-Jun phosphorylation was examined by immunoblotting with anti-phospho-c-Jun(S63) antibody. The same membrane was reprobed with a general anti-c-Jun antibody. Very little c-Jun ubiquitination was seen in the absence of Itch (see panel A). (FIG. 12E)

### EXAMPLE 14

### Costimulation of T Cells Accelerates JunB Degradation

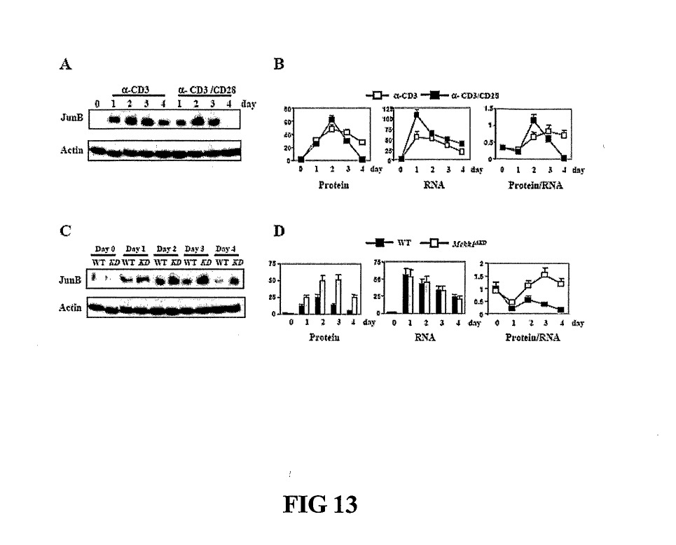

In this example, the biological effects of TCR activation on JunB expression was evaluated. CD4⁺ T cells were incubated with either anti-CD3 alone or anti-CD3+anti-CD28 for several days. The mRNA and protein levels of JunB were examined at different time points and the ratios of protein to RNA were compared. Both conditions induced the expression of JunB mRNA and protein in naïve CD4⁺ T cells (FIG. 13A, B). Although the level of JunB mRNA at day 1 was higher in cells treated with anti-CD3+anti-CD28 than in cells exposed to anti-CD3 alone, the protein levels were similar. After 4 days of stimulation cells exposed to anti-CD3+anti-CD28 hardly contained any JunB protein, while JunB was readily detected in cells exposed to anti-CD3 alone (FIG. 13A). By contrast, the mRNA levels of JunB were similar after 4 days of stimulation with either anti-CD3 or anti-CD3+anti-CD28 (FIG. 13B). These results show that although co-stimulation of T cells results in induction of JunB, it accelerates the turnover of the protein relative to cells exposed to anti-CD3 alone.

JunB mRNA and protein accumulation in CD4⁺ primary T cells from WT and Mekk1^{ΔKD} mice stimulated with anti-CD3+anti-CD28 were also compared. Whereas the levels of JunB mRNA were similar in T cells of the two genotypes, the levels of JunB protein, already after one day of stimulation, were higher in the mutant cells (FIG. 13C, D).

### Co-Stimulation of T Cells Enhancing JunB Turnover

A. CD4⁺ T cells were incubated with anti-CD3 (10 μg) alone or anti-CD3 (10 μg)+anti-CD28 (1 μg). At the indicated times (days), cell extracts were prepared and the levels of JunB were examined by immunoblotting. (FIG. 13A)
B. CD4⁺ T cells were stimulated as above. Levels of JunB protein were quantitated by immunoblotting, whereas JunB mRNA levels were quantitated by Real-time PCR. The relative amounts of JunB protein or mRNA at day 0 were given an arbitrary value of 1.0. Results are averages of 3 experiments done in duplicates. (FIG. 13B)
C. WT and Mekk1^{ΔKD} CD4⁺ T cells were stimulated with anti-CD3+anti-CD28. At the indicated time points, cell extracts were prepared and JunB expression examined as above. (FIG. 13C)
D. WT and Mekk1^{ΔKD} CD4⁺ T cells were treated as above and the levels of JunB mRNA and protein were examined. The results are averages of 3 experiments done in duplicates. (FIG. 13D)

These data highlight differences in JunB protein levels as seen after 4 days of stimulation. While it is not intended that the present invention be limited by any specific mechanism these results are consistent with an accelerated turnover of JunB in co-stimulated T cells that is dependent on MEKK1 catalytic activity.

### EXAMPLE 15

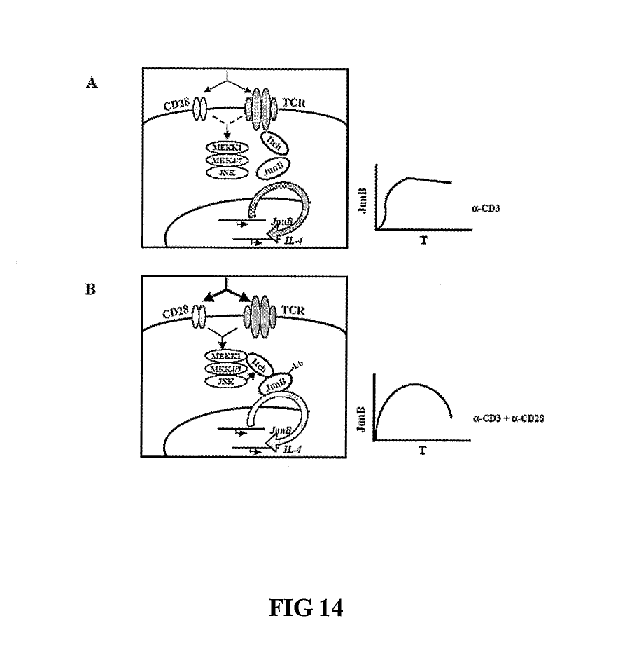

The results described above show a novel signaling pathway that leads to down-regulation of Jun/AP-1 activity in T cells in response to TCR and CD28 engagement by accelerating the turnover of JunB and c-Jun (FIG. 14).

### JunB Turnover According to the Strength of the T Cell Activating Stimulus

A model summarizing inventors results and illustrating a mechanism through which the MEKK1-JNK signaling pathway can modulate the extent of Th2 differentiation. The MEKK1-JNK signaling cascade regulates JunB turnover and thereby modulates Th2 cell differentiation according to the strength of the T cell activating stimulus by enhancing Itch activity. (FIG. 14)

Co-Stimulation of T Cells with Anti-CD3+Anti-CD28

(14B) results in more JNK activity and accelerates the turnover of JunB relative to cells treated with anti-CD3 alone (14A). Enhanced turnover of JunB attenuates the expression of IL-4 and negatively affects Th2 differentiation. Either stimulus results in initial induction of JunB.

### Discussion

Previous studies revealed that AP-1 proteins play an important role in induction of T cell cytokine genes both by direct binding to AP-1 sites in their promoters and by enabling NF-AT proteins to recognize low affinity sites (Rao et al. (1997) Annu Rev Immunol 15, 707-747; Rooney et al. (1995) Immunity 2, 473-483). Furthermore, the transcriptional activities of both c-Jun and JunB were shown to be enhanced by JNK-mediated phosphorylation in T cells (Li et al. (1999) Embo J 18, 420-432; Su et al. (1994) Cell 77, 727-736).

As evidenced by the data in the Experimental section above elimination of MEKK1 catalytic activity or JNK inhibition presents the same effect on T cell proliferation and Th2 cytokine production as seen after elimination of JNK1/2 or MKK7 or overexpression of JunB. These data provide both biochemical and genetic evidence that the inhibitory effect of MEKK1 on T cell proliferation and Th2 cytokine gene expression is via JNK. These data also document the biochemical pathway through which MEKK1-dependent JNK activation exerts this negative regulatory activity.

Moreover, CD4⁺ T cells lacking MEKK1 catalytic activity or T cells treated with JNK inhibitors accumulate both c-Jun and JunB, while they continue to express normal levels of other transcription factors involved in Th2 cytokine expression or JunD. The accumulation of c-Jun and JunB is shown to be due to their decreased degradation. This effect of disruption of the MEKK1-JNK cascade on Jun protein turnover and cytokine production is identical to that of inactivation of the E3 ubiquitin ligase Itch.

Previous studies revealed that Itchy mice, expressing an inactive form of Itch, overproduce Th2 cytokines and accumulate c-Jun and JunB in their T cells, while expressing normal levels of JunD or other transcription factors (Fang et al. (2002) Nat Immunol 3, 281-287). As evidenced by the data in the Experimental section above, T cell activation enhances the E3 ligase activity of Itch and that JNK activation, which is compromised in Mekk1^{ΔKD} T cells, is required for this enhancement. These data demonstrate that enhanced Itch activity in response to TCR and CD28 co-ligation.

As evidenced by the data in the Experimental section above, an important function of the MEKK1-JNK cascade in T cells undergoing robust activation brought about by strong co-stimulation of both TCR and CD28 is to enhance the Itch-dependent degradation of important regulatory proteins, such as JunB. As JunB is required for Th2, but not Th1, cytokine production (Hartenstein et al. (2002) Embo J 21, 6321-6329), the overall effect of this pathway is to attenuate the production of IL-4, the Th2 cytokine (Debonneville et al. (2001) Embo J 20, 7052-7059; Paul et al. (1994) Cell 76, 241-251), and thereby decrease the polarization of naïve Th cells towards the Th2 phenotype (FIG. 14). MEKK1 catalytic activity is not required for JunB induction and the strength of T cell activation has little effect on the level of JunB mRNA. Almost as much JunB mRNA accumulates after engagement of TCR alone as after co-ligation of TCR and CD28 (FIG. 13). The data in the Experimental section above provides a physiologically relevant example that ubiquitin-dependent protein turnover can be regulated via substrate protein phosphorylation but also through modulation of the catalytic activity of a substrate-specific E3 ligase. These data provide evidence that the catalytic activity of Itch is positively regulated. These data also show that the Itch-dependent ubiquitination of c-Jun is not affected by elimination of the JNK phosphorylation sites and that T cells from c-Jun^{AA} mice, in which serines 63 and 73 of c-Jun [the JNK phosphorylation sites (Smeal et al. (1991) Nature 354, 494-496)], were replaced with alanines (Behrens et al., (1999) Nat Genet 21, 326-329), express lower levels of c-Jun relative to WT counterparts. These data also show that disruption of the PHD domain of MEKK1 abolishes Itch-dependent Jun ubiquitination.

These data suggest that, in one embodiment, the biological function of the pathway through which MEKK1/JNK activation enhances Itch activity and leads to accelerated Itch-dependent JunB turnover in T cells is to attenuate the polarization of naïve Th cells towards the Th2 phenotype following strong T cell stimulation, as occurs after co-engagement of TCR and CD28.

### EXAMPLE 16

Increased Th2 Cytokine Production in Mekk1^{ΔKD} Cells with Reduced JNK Activity

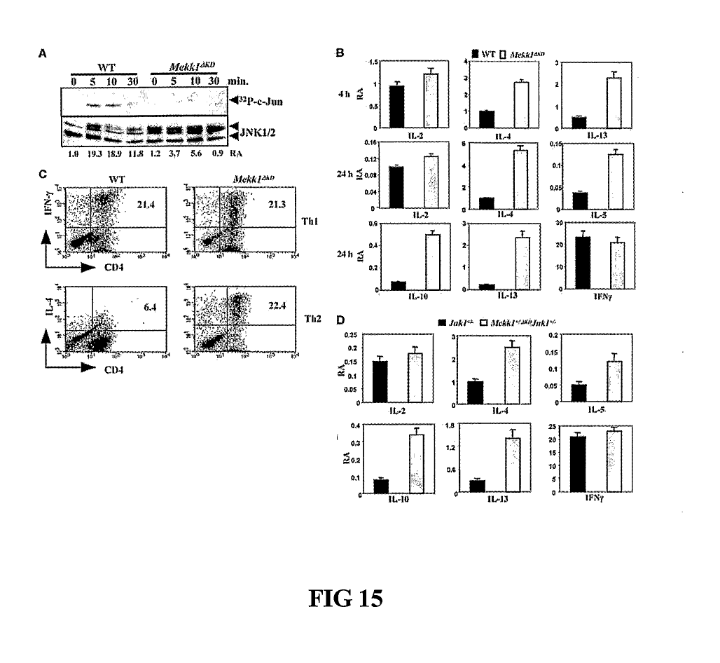

To understand how JNK activation regulates Th2 cytokine expression the T cells from Mekk1^{KD} mice that express an inactive form of MEKK1 were examined (Zhang et al. (2003) Embo J 22, 4443), a MAP kinase (MAPK) kinase kinase (MAP3K) that is a potent activator of JNK signaling (Minden et al. (1994) Science 266, 1719). Mekk1^{ΔKD} mice are viable (Zhang et al. (2003) Embo J 22, 4443) without any obvious defect in generation or survival of T cells or their intrathymic differentiation into CD4⁺ and CD8⁺ subsets (FIG. 8C). Yet, T cells from these mice exhibit reduced JNK activation following engagement of the T cell receptor (TCR) and the CD28 auxiliary receptor (FIG. 15A). Mekk1^{ΔKD} peripheral T cells and thymocytes also hyperproliferated in response to stimulation with anti-CD3+anti-CD28 antibodies (FIG. 8B) and within 4 hrs of receptor engagement expressed larger amounts of IL-4 and IL-13 mRNAs relative to those in wild type (WT) cells, while expressing normal amounts of IL-2 mRNA (FIG. 15B). IL-4, as the master regulator of Th2 effector T cell differentiation, can induce expression of other cytokines including IL-5, IL-10, and IL-13 (Paul et al. (1994) Cell 76, 241). Indeed, after 24 hrs activated Mekk1^{ΔKD} T cells expressed increased amounts of IL-5, IL-10, and IL-13 mRNAs in addition to IL-4 mRNA (FIG. 15B). The mRNAs for IL-2 and the Th1 cytokine interferon γ (IFNγ) remained unchanged. The inventors also cultured naïve WT or Mekk1^{ΔKD} CD4+ T cells under Th1- or Th2-polarization conditions (Paul et al. (1994) Cell 76, 241; Murphy et al. (2000) Annu Rev Immunol 18, 451) to examine their differentiation into effector cells. Due to the BL6×C129 genetic background (20), WT Th2 cells produced relatively small amounts of IL-4, but their amounts were increased in Mekk1^{ΔKD} Th2 cells (FIG. 15C). Production of IFNγ by Th1 cells remained unchanged.

To examine whether the effect of MEKK1 on Th2 cytokine production is JNK-dependent Jnk1^{-/-} mice were crossed with Mekk1^{ΔKD} mice. Mekk1^{+/ΔKD}Jnk1^{±} CD4⁺ T cells expressed increased amounts of IL-4, IL-5, IL-10, and IL-13 mRNAs relative to those in Jnk1^{±} (FIG. 15D) or Mekk1^{+/ΔKD} (21) T cells, indicating JNK1 dependence.
(A) WT and Mekk1^{ΔKD} T cells were incubated with anti-CD3 and anti-CD28. When indicated, JNK activity was measured by an immunecomplex kinase assay with GST-c-Jun as the substrate. Phosphorylated c-Jun was detected by autoradiography and quantitated with a PhosphorImager. The amount of immunoprecipitated JNKs was determined by immunoblotting. Relative JNK activity (RA) at t=0 was given an arbitrary value of 1.0. (FIG. 15A)
(B) CD4⁺ T cells from WT and Mekk1^{ΔKD} mice were incubated with anti-CD3 and anti-CD28. Cytokine mRNA levels were quantitated by Real-time PCR at the indicated times and normalized to amounts of cyclophilin A mRNA. The relative amount (RA) of IL-4 mRNA in WT cells at each time point was given an arbitrary value of 1.0. (FIG. 15B)
(C) CD4⁺ T cells were cultured under Th1- or Th2-polarizing conditions for 7 days. Th subsets were restimulated with anti-CD3 and anti-CD28 and analyzed for cytokine expression by flow cytometry after 6 hours. (FIG. 15C)
(D) CD4⁺ T cells from Jnk1^{±}and Mekk1^{+/ΔKD}Jnk1^{±} mice were stimulated with anti-CD3 and anti-CD28 and cytokine mRNAs were quantitated as in (B) after 24 hours. (FIG. 15D)

As shown above the loss of MEKK1 and JNK1 does not affect IL-2 mRNA levels. The elevated Th2 cytokine response of Mekk1^{ΔKD} CD4⁺ T cells was IL-4-dependent, as it was absent in Mekk1^{ΔKD}IL-4^{-/-} T cells (FIG. 9D).

### EXAMPLE 17

A Decreased Turnover of JunB and c-Jun After Inactivation of MEKK1 or JNK

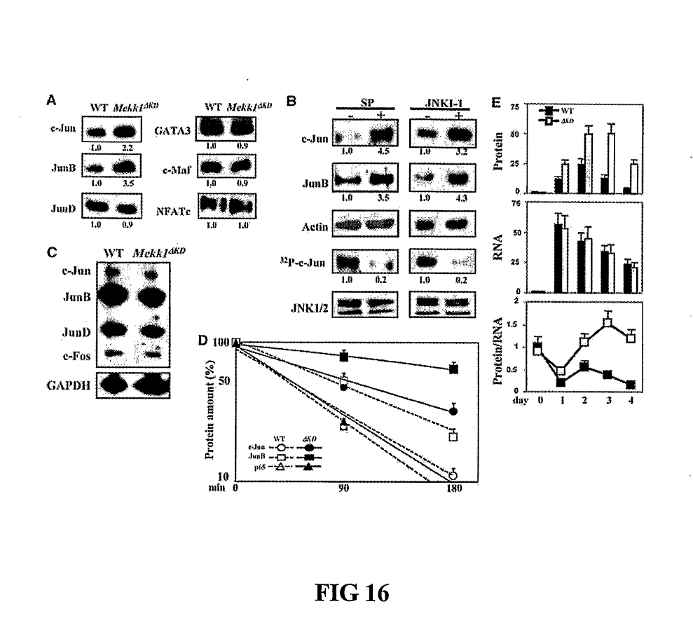

Multiple transcription factors, including NF-AT, GATA-3, c-Maf, and JunB, regulate IL-4 gene expression (Murphy et al. (2000) Annu Rev Immunol 18, 451). The amounts of these transcription factors in WT and Mekk1^{ΔKD} T cells were examined. Both c-Jun and JunB were increased in activated Mekk1^{ΔKD} CD4⁺ T cells, whereas amounts of JunD, another family member, as well as GATA-3, c-Maf, NF-ATc1 and NF-ATc2, were unchanged (FIG. 16A). The effect on c-Jun and JunB resulted from loss of JNK activity, because two different JNK inhibitors -SP600125 (23) and JNKI-1, a peptide inhibitor based on the c-Jun docking site for JNK (24, 25)-, also increased c-Jun and JunB expression (FIG. 16B). No such effects were observed with the p38 inhibitor SB202190 (Gao et al., Unpublished data). Elevated c-Jun and JunB expression was also found in Jnk1^{-/-} T cells, whereas Jnk2^{-/-} T cells showed a slight reduction. These results indicate that a JNK1 deficiency rather than a JNK2 deficiency is responsible for the altered expression of c-Jun and JunB in Mekk1^{ΔKD} T cells.

Despite the change in protein abundance, the amounts of c-Jun and JunB mRNAs remained unchanged (FIG. 16C). The inventors also examined whether MEKK1 and JNK promoted c-Jun and JunB turnover. In pulse-chase experiments, in WT T cells, newly synthesized c-Jun and JunB turned-over with half-lives (t_{1/2}) of 59 and 78 min, respectively, but were more stable in Mekk1^{ΔKD} cells, with t_{1/2}s of 109 and 280 min, respectively (FIG. 16D). The turnover of RelA (p65) remained unaltered. After engagement of TCR and CD28 in WT and Mekk1^{ΔKD} T cells the amounts of JunB mRNA were very similar, but the amounts of JunB protein, after 1 to 4 days of stimulation, were greater in the mutant cells (FIG. 16E).
(A) WT and Mekk1^{ΔKD} CD4⁺ T cells were stimulated with anti-CD3 and anti-CD28 for 24 hours. Cell extracts were prepared and the amounts of various transcription factors were measured by immunoblotting and densitometry. The amount of each protein in WT cells was given an arbitrary value of 1.0. (FIG. 16A)
(B) WT CD4⁺ T cells were stimulated as above in the absence or presence of JNK inhibitors (SP600125 or JNKI-1) for 24 hours. Jun protein levels were analyzed as above and JNK activity was measured by an immunecomplex kinase assay. (FIG. 16B)
(C) The mRNA amounts of Jun family members and c-Fos in cells from (A) were measured by RNase protection. (FIG. 16C)
(D) WT and Mekk1^{ΔKD} T cells were pulse-labeled with [³⁵S] amino acids and chased with non-labeled amino acids. Proteins were immunoprecipitated at the indicated times post-labeling, separated by SDS-PAGE and analyzed by autoradiography and phosphorimaging. The relative amounts of each [³⁵S]-labeled protein at t=0 were considered to be 100%. (FIG. 16D)
(E) WT CD4⁺ T cells were stimulated with anti-CD3 and anti-CD28. At the indicated time points, the relative levels of JunB mRNA and protein were quantitated as in (A) and (C). (FIG. 16E)

### EXAMPLE 18

A MEKK1-JNK Cascade Promotes c-Jun and JunB Ubiquitination by Enhancing Itch Activity

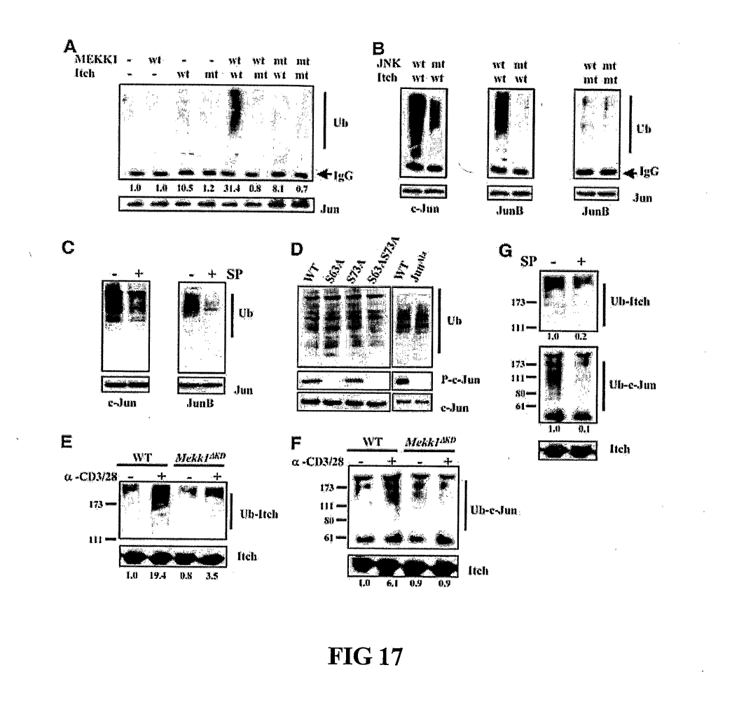

Transfected 293T cells were examined because of difficulties in detecting endogenously ubiquitinated proteins in primary T cells. Ectopic expression of MEKK1 in 293T cells promoted polyubiquitination of both c-Jun (Gao et al., Unpublished data) and JunB (FIG. 17A) in an Itch-dependent manner. Overexpression of kinase-deleted MEKK1 did not enhance Itch-dependent Jun polyubiquitination (FIG. 17A). Itch-dependent Jun ubiquitination was enhanced by a constitutively active JNKK2-JNK1 fusion protein, but not by an inactive version (FIG. 17B). JNK-enhanced ubiquitination correlated with accelerated JunB degradation. Treatment with a JNK inhibitor reduced the extent of Itch+MEKK1-induced polyubiquitination of c-Jun and JunB (FIG. 17C). This effect of JNK, however, appeared to be independent of JNK-mediated c-Jun phosphorylation because mutant versions of c-Jun lacking its JNK phosphorylation sites (Musti et al. (1997) Science 275, 400; Smeal et al. (1991) Nature 354, 494) were ubiquitinated as efficiently as WT c-Jun in cells overexpressing Itch and WT MEKK1 (FIG. 17D).

Unlike F box-containing E3 ligases, HECT domain ligases are thought to recognize their substrates independently of their phosphorylation. The inventors then examined whether JNK activation enhances Jun ubiquitination by modulating the activity of Itch. Itch undergoes self-ubiquitination, and this activity was enhanced if it was isolated from WT T cells activated with antibodies to CD3 and CD28 (FIG. 17E). Little enhancement of Itch self-ubiquitination was observed after activation of Mekk1^{ΔKD} T cells (FIG. 17E). The ability of Itch to promote ubiquitination of a GST-c-Jun substrate was enhanced in response to T cell activation and this response was also diminished in Mekk1^{ΔKD} cells (FIG. 17F). Both Itch self-ubiquitination and its ability to promote c-Jun polyubiquitination largely dependent on incubation with both E1 and E2 (Ubc7) enzymes and were reduced in activated T cells that were treated with a JNK inhibitor (FIG. 17G).
(A) 293T cells were transiently transfected with plasmids encoding HA-tagged ubiquitin, Myc-tagged JunB, WT or a kinase domain-deleted (mt) MEKK1, and WT or catalytically inactive (mt) Itch. Ubiquitin conjugation was examined by immunoblotting with an antibody to HA and quantitated by densitometry. (FIG. 17A)
(B) 293T cells were transfected with HA-ubiquitin, Myc-tagged c-Jun or JunB, WT or catalytically inactive (mt) Itch, and WT or inactive (mt) JNKK2-JNK1 fusion proteins. Jun ubiquitination was examined as above. (FIG. 17B)
(C) 293T cells were transfected with c-Jun or JunB, HA-ubiquitin, Itch and MEKK1. Cells were incubated with or without JNK inhibitor (SP600125) for 24 hours and analyzed as above. (FIG. 17C)
(D) 293T cells were transfected as above with WT or phosphorylation-deficient c-Jun constructs, Itch and MEKK1. Ubiquitin conjugation was analyzed as above and c-Jun phosphorylation was examined by immunoblotting. (FIG. 17D)
(E) WT and Mekk1^{ΔKD} T cells were left unstimulated or stimulated with anti-CD3 and anti-CD28 for 15 min. Itch was immunoprecipitated and incubated with ubiquitin, E1, E2, and ATP. Itch self-ubiquitination was analyzed by immunoblotting with antibody to ubiquitin and quantitated by densitometry. (FIG. 17E)
(F) T cells were treated as above. Itch immunecomplexes were isolated and incubated with ubiquitin, E1, E2, ATP and purified GST-c-Jun. c-Jun ubiquitination was analyzed by immunoblotting. (FIG. 17F)
(G) WT T cells were stimulated as above in the absence or presence of SP600125. In vitro ubiquitination assays using immunoprecipitated Itch as the E3 with or without GST-c-Jun as the substrate were done as above. (FIG. 17G)

### EXAMPLE 19

### Increased E3 Activity of Itch After JNK-Mediated Phosphorylation

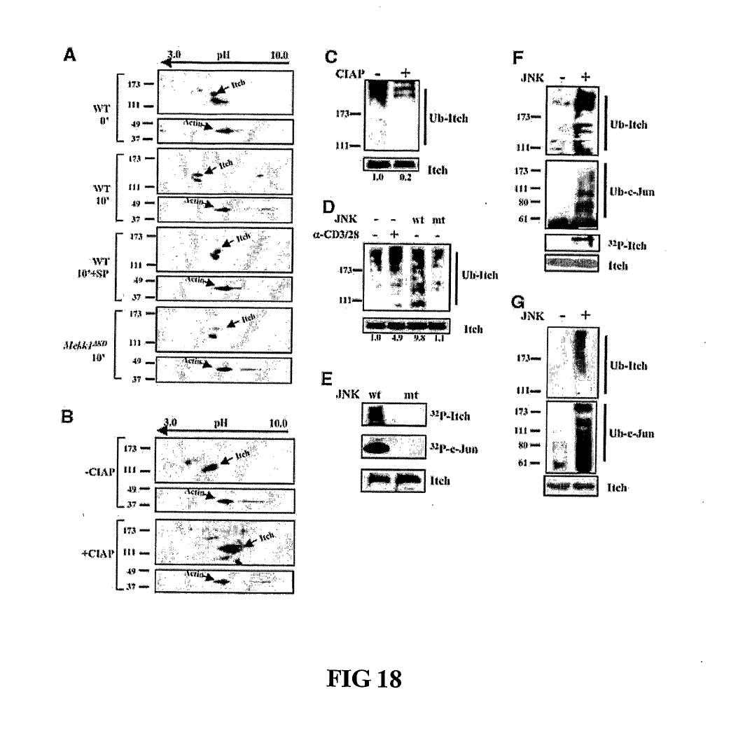

To examine whether Itch is a target for JNK-mediated phosphorylation, proteins were separated from non-activated and activated T cells by two dimensional (2D) gel electrophoresis and transferred them to membranes. Following T cell activation, Itch became more negatively charged and displayed a lower isoelectric point (FIG. 18A). These changes are consistent with increased Itch phosphorylation and were reversed by calf intestine alkaline phosphatase (CIAP) (FIG. 18B). The kinetics of Itch phosphorylation correlated with those of JNK activation. Furthermore, Itch phosphorylation was reduced by treatment with a JNK inhibitor and compared to WT cells, less TCR-induced Itch phosphorylation was observed in Mekk1^{ΔKD} cells (FIG. 18A). Similarly, Itch self-ubiquitination and c-Jun polyubiquitination were reduced after CIAP treatment of isolated Itch (FIG. 18C and (Gao et al., Unpublished data)). Moreover, incubation of Itch from non-stimulated T cells with activated JNK1 enhanced its self-ubiquitination, but incubation with inactive JNK1 had no effect (FIG. 18D). Incubation of Itch with active JNK1 also led to its efficient phosphorylation (FIG. 18E). Incubation of in vitro translated Itch with JNK1 also increased its self-ubiquitination and its ability to promote c-Jun polyubiquitination (FIG. 18F). Similar results were obtained with recombinant Itch produced in E. coli (FIG. 18G). Consistent with the changes in c-Jun and JunB expression seen in Jnk1^{-/-} and Jnk2^{-/-} T cells. JNK1 is a more efficient Itch kinase than JNK2 and as a result is a more potent activator of Itch. The highly efficient phosphorylation of Itch by JNK1 is due to the presence of a JNK docking site, whose mutational inactivation prevents Itch phosphorylation and activation by JNK1 (Gao et al., Unpublished data).
(A) Proteins from non-stimulated (0′) and activated (10′) WT T cells incubated without or with SP600125 were resolved by 2D gel electrophoresis, transferred to membranes, and immunoblotted with antibodies to Itch and actin. Itch migration in activated Mekk1^{ΔKD} cells was similarly analyzed. (FIG. 18A)
(B) Extracts from activated WT T cells were incubated without or with CIAP before 2D gel electrophoresis and immunoblotting as above. (FIG. 18B)
(C) Itch was immunoprecipitated from the extracts used in (B) and incubated with ubiquitin, E1, E2, and ATP. Itch ubiquitination was analyzed by immunoblotting with anti-ubiquitin antibody and densitometry. (FIG. 18C)
(D) Immunoprecipitated Itch from non-stimulated T cells was incubated with active (wt) or inactive (mt) JNK1 and JNKK1. Itch ubiquitination was analyzed as above and quantitated by densitometry. (FIG. 18D)
(E) Immunoprecipitated Itch from non-stimulated T cells was incubated with active or inactive JNK1 and JNKK1 in the presence of [γ-³²P]ATP. GST-c-Jun was included as a positive control. Protein phosphorylation was analyzed by autoradiography. The same membrane was probed with antibody to Itch. In vitro translated Itch (F) or a recombinant GST-Itch (G) was incubated with JNK1 and JNKK1. Itch and c-Jun polyubiquitination were analyzed as above. Itch phosphorylation was analyzed by autoradiography. (FIG. 18E)

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the invention.

1. A method for identifying a test agent as reducing the level of differentiation of T cells into Th1 cells comprising reducing MEKK1 catalytic activity in said T cells.

2. The method of claim 1, wherein reducing of said MEKK1 catalytic activity comprises increasing the level of differentiation of said T cells into Th2 cells.

3. The method of claim 1, wherein the reducing of said MEKK1 catalytic activity comprises increasing the level of one or more Th2 cell cytokine that is produced by said T cells.

4. The method of claim 3, wherein the increased level of said Th2 cytokine occurs in the absence of an increase in the level of one or more Th1 cytokine.

5. The method of claim 4, wherein said Th1 cytokine is chosen from one or more of interferon-gamma and Interleukin-2.

6. The method of claim 3, wherein said Th2 cytokine is chosen from one or more of Interleukin-4, Interleukin-5, Interleukin-10, and Interleukin-13.

7. The method of claim 3, wherein said increasing the level of said Th2 cell cytokine comprises increasing the level of mRNA encoding said Th2 cytokine.

8. The method of claim 7, wherein said mRNA encoding said Th2 cytokine is increased 5 fold.

9. The method of claim 3, wherein said reducing of said MEKK1 catalytic activity comprises increasing the level of proliferation of Th2 cells that differentiate from said T cells.

10. The method of claim 3, wherein said reducing of said MEKK1 catalytic activity comprises introducing a mutation in the gene encoding MEKK1.

11. The methods of claim 3, wherein said T cells comprise thymocyte cells.

12. The methods of claim 3, wherein said T cells comprise splenocyte cells.

13. The methods of claim 3, wherein said T cells are in vitro.

14. The methods of claim 13, wherein said T cells are in vivo in an animal.

15. The methods of claim 14, wherein said animal is human.

16. The method of claim 15, wherein said human is chosen from one or more of a human that is:
(a) suspected of having a Th1-mediated disease;
(b) not suspected of having a Th1-mediated disease;
(c) suspected of being capable of developing a Th1-mediated disease; and
(d) suspected of not being capable of developing a Th1-mediated disease.

17. The methods of claim 16, wherein said Th1-mediated disease is chosen from multiple sclerosis, type 1 diabetes, autoimmune thyroiditis, and rheumatoid arthritis.

18-19. (canceled)

20. A method for increasing Th2 cytokine levels produced by T cells, comprising:
(a) providing:
(i) an inhibitor of ITCH;
(ii) T cells; and
(iii) test agent; and
(b) contacting said T cells in the presence of said test agent to produce contacted T cells and in the absence of said test agent to produce control T cells; and
(c) detecting reduced activity of ITCH in said contacted T cells compared to ITCH in said control T cells, wherein said detecting identifies said test agent as increasing Th2 cytokine levels produced by T cells.

21. A method for increasing Th2 cytokine levels produced by T cells, comprising:
(a) providing:
(i) a kinase inhibitor, wherein said kinase is one or more of MEKK1 and JNK1;
(ii) T cells;
(iii) test agent; and
(b) contacting said T cells in the presence of said test agent to produce contacted T cells and in the absence of said test agent to produce control T cells; and
(c) detecting reduced activity of said kinase in said contacted T cells compared to said kinase in said control T cells, wherein said detecting identifies said test agent as increasing Th2 cytokine levels produced by T cells.

22. The method of claim 20, further comprising, (d) identifying said test agent as increasing the level of a Th2 cytokine.

23. The method of claim 22, wherein said Th2 cytokine is one or more of Interleukin-4, Interleukin-5, Interleukin-10, and Interleukin-13.

24. The method of claim 20, further comprising, (d) identifying said test agent as decreasing the level of a Th1 cytokine.

25. The method of claim 21, wherein said kinase inhibitor comprises SB600125.

26-63. (canceled)

64. The method of claim 21, further comprising, (d) identifying said test agent as increasing the level of a Th2 cytokine.

65. The method of claim 64, wherein said Th2 cytokine is one or more of Interleukin-4, Interleukin-5, Interleukin-10, and Interleukin-13.

66. The method of claim 21, further comprising, (d) identifying said test agent as decreasing the level of a Th1 cytokine.


SEQUENCE LISTING (4 sequences)

SEQ ID NO: 1 - DNA, 20 bp, Artificial Sequence
  Synthetic
gttgctgaaaccaagggaaa

SEQ ID NO: 2 - DNA, 20 bp, Artificial Sequence
  Synthetic
tgaaaggccgattatggtgt

SEQ ID NO: 3 - DNA, 20 bp, Artificial Sequence
  Synthetic
tcaacccccagctagttgtc

SEQ ID NO: 4 - DNA, 20 bp, Artificial Sequence
  Synthetic
aaatatgcgaagcaccttgg